(19) United States
(12) Patent Application Publication
(10) Pub. No.: US 2009/0227575 A1
(43) Pub. Date: Sep. 10, 2009

(54) 7H-PYRROLO[2,3-H]QUINAZOLINE COMPOUNDS, THEIR USE AS mTOR KINASE AND PI3 KINASE INHIBITORS, AND THEIR SYNTHESIS

(75) Inventors: Aranapakam Mudumbai Venkatesan, Rego Park, NY (US); Zecheng Chen, New City, NY (US); Osvaldo Dos Santos, Astoria, NY (US); Natasja Brooijmans, New York, NY (US); Ariamala Gopalsamy, Mahwah, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/397,590

(22) Filed: Mar. 4, 2009

Related U.S. Application Data
(60) Provisional application No. 61/033,464, filed on Mar. 4, 2008.

(51) Int. Cl. A61K 31/5377; C07D 487/04; A61P 35/00

(52) U.S. Cl. 514/232.8; 544/115

(57) ABSTRACT

A 7H-pyrrolo[2,3-h]quinazoline compound of the formula I

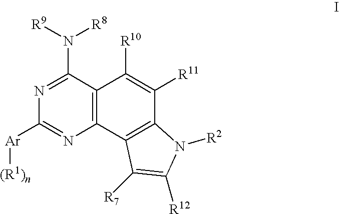

wherein Ar, R¹, R², R⁷, R⁸, R⁹, R¹⁰, R¹¹, R¹², and n are as defined in the specification, and methods for making same.

### FIELD OF THE INVENTION

The invention relates to 7H-pyrrolo[2,3-h]quinazoline compounds, compositions comprising a 7H-pyrrolo[2,3-h]quinazoline compound, methods of synthesizing these compounds, and methods for treating mTOR-related diseases. The invention also relates to methods for treating PI3K-related diseases. The invention also includes intermediates useful for making the active compounds.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267(1997)].

The class Ia PI3K subtype has been most extensively investigated to date. Within the class la subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. c Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR enzymes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with an IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth which originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzymes with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the α isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over-activation of the upstream pathway would normally cause mTOR kinase to be over activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. Everolimus is in a phase II clinical study for patients with Stage IV Malignant Melanoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

The most recently described PI3K family member was identified in human cells and named human or hSMG-1. Yamashita (Genes Dev. 2001 15: 2215-2228) characterized two isoforms of hSMG-1 proteins, p430 and p400, which are expressed in various cell lines of human, monkey, rat, and mouse. Yamashita's p400 hSMG-1 isoform is a 3529-amino-acid protein of 396,040 Daltons. Brumbaugh (Molecular Cell, Volume 14, Issue 5, 4 Jun. 2004, Pages 585-598) isolated a 3521 amino acid polypeptide with a deduced molecular mass of 395 kDa. Brumbaugh's hSMG-1 is eight amino acids shorter at the amino terminus than the protein isolated by Yamashita. Both hUpf1 and p53 are physiological targets for hSMG-1 in intact cells. Rapamycin in the presence of purified recombinant FKBP12 does not inhibit the kinase activity of hSMG-1. Wortmannin, the modified steroidal anti-infective agent, and the purine caffeine inhibit the kinase activity of hSMG-1 with IC₅₀ values of ˜60 nM and 0.3 mM, respectively. However, these are non-specific protein kinase inhibitors.

Specific inhibition of hSMG-1 is a potential therapeutic strategy because inhibitors of hSMG-1 cause the accumulation of truncated p53 proteins from a premature translation termination codon (PTC) allele, as well as the increase in the level of mRNA with PTC, opening the possibility of the above strategy by specifically suppressing nonsense-mediated mRNA decay (NMD) through the inhibition of hSMG-1.

One-fourth of all mutations in human genetic diseases and cancers are of the type that can target the corresponding mRNA for NMD. Although NMD protects cells against deleterious gain-of-function mutations caused by the dominant negative effects of aberrant truncated proteins, there are some cases in which the truncated protein does not show such an effect, rather, it retains residual activity and can compensate for the normal gene function. Thus, the specific inhibition of NMD may provide a novel therapeutic strategy based on the type of mutation rather than on the gene in which the mutation resides.

The inhibitors of SMG-1 can rescue the synthesis of mature proteins through two independent mechanisms (i.e., the inhibition of NMD to increase the mRNA level and the suppression of translational termination that leads to the synthesis of a read-through mature protein product). In this sense, the specific inhibitors of hSMG-1 will be of potential therapeutic importance for all the genetic diseases associated with PTC mutations.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides compounds of the Formula I:

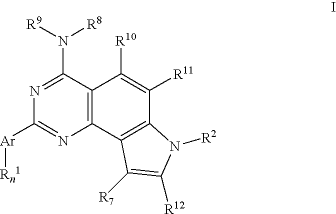

or pharmaceutically acceptable salt thereof, wherein the constituent variables are as defined below.

In one aspect, the invention provides compounds of the Formula II:

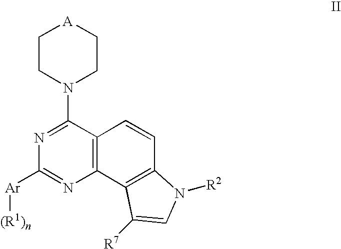

or pharmaceutically acceptable salt thereof, wherein the constituent variables are as defined below.

In other aspects, the invention provides pharmaceutical compositions comprising compounds or pharmaceutically acceptable salts of compounds of the present formula II.

In one aspect, the compounds or pharmaceutically acceptable salts of the compounds of the present formula II are useful as mTOR inhibitors.

In one aspect, the compounds or pharmaceutically acceptable salts of the compounds of the present formula II are useful as PI3K inhibitors.

In one aspect, the invention provides methods for treating an mTOR-related disorder, comprising administering to a mammal in need thereof, the compounds or pharmaceutically acceptable salts of compounds of the present formula II in an amount effective to treat an mTOR-related disorder.

In one aspect, the invention provides methods for treating a PI3K-related disorder, comprising administering to a mammal in need thereof the compounds or pharmaceutically acceptable salts of compounds of the present formula II in an amount effective to treat a PI3K-related disorder.

In other aspects, the invention provides further methods of synthesizing the compounds or pharmaceutically acceptable salts of compounds of the present formula II.

In other aspects, the invention provides intermediates of Formula III useful in synthesizing compounds of Formula II:

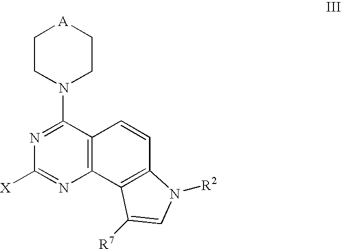

wherein the constituent variables are as defined below.

### DETAILED DESCRIPTION OF THE INVENTION

In one aspect, the invention provides compounds of the Formula I:

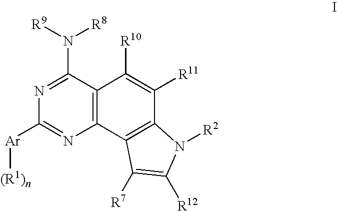

or pharmaceutically acceptable salt thereof, wherein
Ar is phenyl, naphthyl, or nitrogen-containing mono- or bicyclic C₁-C₉heteroaryl;
R¹ is independently NR³R⁴; NHC(O)NR³R⁴; —NHC(O)OR⁵; R⁵C(O)NH—; R⁵C(O)—; R⁵S(O)ₚNH—; CHO; C₁-C₆hydroxylalkyl-; C₃-C₆hydroxylalkenyl-; (C₆-C₁₄aryl)alkyl optionally substituted by hydroxyl; (C₆-C₁₄aryl)alkyl-O—; (C₁-C₆alkoxy)carbonyl; HO₂C—; R³R⁴NC(O)—; N≡C—; carboxyamido(C₁-C₆)alkyl-; hydroxyl; halo; C₁-C₆alkoxy optionally substituted with from 1 to 3 substituents independently selected from C₁-C₆alkoxy, —NH₂, —NH(C₁-C₆alkyl), and —N(C₁-C₆alkyl)(C₁-C₆alkyl); —NH(SO₂)NH—(C₁-C₆alkyl); —NH(SO₂)N—(C₁-C₆alkyl)(C₁-C₆alkyl); —O-heterocycle optionally substituted by C₁-C₆alkyl; H₂NC₁—C₆alkyleneSO₂—; (C₁-C₆alkyl)NHC₁-C₆alkyleneSO₂—_{;} (C₁-C₆alkyl)(C₁-C₆alkyl)NC₁-C₆alkyleneSO₂—; heterocyclyl(C₁-C₆alkyl)SO₂—; carboxyamido(C₁-C₆)alkyl-C(O)—; heterocycle-C(O)—C₁-C₆alkylene-C(O)—; R³R⁴NSO₂C₁-C₆alkylene-C(O)—; or —SO₂NR³R⁴;
n is 0, 1, 2, 3, 4, or 5;
each p is independently 1 or 2;
R³ and R⁴ are each independently:
   (a). H;
   (b). C₁-C₆alkyl optionally substituted with from 1 to 3 substituents independently selected from:
      (i). —NH₂,
      (ii). —NH(C₁-C₆alkyl),
      (iii). —N(C₁-C₆alkyl)(C₁-C₆alkyl),
      (iv). C₁-C₆alkoxy,
      (v). C₃-C₈cycloalkyl,
      (vi). C₃-C₈cycloalkenyl,
      (vii). halo,
      (viii). and C₁-C₉heteroaryl;
   (c). C₁-C₉heteroaryl optionally substituted with from 1 to 3 substituents independently selected from:
      (ix). C₁-C₆alkyl,
      (x). C₁-C₆aminoalkyl-,
      (xi). C₁-C₆hydroxylalkyl-,
      (xii). and C₁-C₉heterocyclyl-;
   (d). heterocyclyl(C₁-C₆alkyl)-;
   (e). (C₁-C₉heterocyclyl)-;
   (f). (C₁-C₉heterocyclyl)-SO₂—;
   (g). C₆-C₁₄aryl optionally substituted with from 1 to 3 substituents independently selected from:
      (xiii). C₁-C₆alkyl,
      (xiv). C₁-C₆alkoxy,
      (xv). C₁-C₆aminoalkyl-,
      (xvi). C₁-C₆hydroxylalkyl-,
      (xvii). C₁-C₆aminoalkyl-NH—,
      (xviii). C₁-C₆hydroxylalkyl-NH—,
      (xix). halo,
      (xx). C₁-C₉heterocyclyl,
      (xxi). (C₁-C₉heteroaryl)-O—,
      (xxii). —(C₁-C₉heterocycle)-O—,
      (xxiii). (C₁-C₉heterocyclyl)-S—,
      (xxiv). (C₁-C₉heterocyclyl)-CO—,
      (xxv). (C₁-C₆alkyl)-NH—C(O)—,
      (xxvi). (C₁-C₆alkyl)(C₁-C₆alkyl)N—C(O)—,
      (xxvii). H₂NNH—C(O)—,
      (xxviii). R⁵—C(O)—,
      (xxix). (C₁-C₆alkyl)-NH—NH—C(O)—,
      (xxx). (C₁-C₆alkyl)(C₁-C₆alkyl)NNH—C(O)—,
      (xxxi). (C₁-C₉heteroaryl)NH—C(O)—,
      (xxxii). (C₆-C₁₄aryl)NH—C(O)—,
      (xxxiii). (C₁-C₆alkyl)-SO₂—,
      (xxxiv). (C₁-C₉heterocyclyl)-SO₂—,
      (xxxv). H₂NS(O)₂—,
      (xxxvi). (C₁-C₆alkyl)NH—SO₂—,
      (xxxvii). (C₁-C₆alkyl)(C₁-C₆alkyl)N—SO₂—,
      (xxxviii). H₂NNHS(O)₂—,
      (xxxix). (C₁-C₆alkyl)NH—NH—SO₂—,
      (xl). (C₁-C₆alkyl)(C₁-C₆alkyl)N—NH—SO₂—,
      (xli). (C₁-C₉heteroaryl)NH—S(O)₂—,
      (xlii). (C₆-C₁₄aryl)NH—S(O)₂—,
      (xliii). and perfluoro(C₁-C₆)alkyl-;
   (h). or C₃-C₈cycloalkyl-;
or R³ and R⁴, when taken together with the nitrogen to which they are attached, can form a 3- to 7-membered nitrogen containing heterocycle wherein up to two of the carbon atoms of the heterocycle can be replaced with —N(R⁶)—, —O—, S, S(O), or —S(O)₂—;
R⁶ is hydrogen or C₁-C₆alkyl;
R⁵ is C₁-C₆alkyl-, C₃-C₈cycloalkyl-, C₁-C₉heteroaryl, or C₆-C₁₄aryl- optionally substituted with from 1 to 3 substituents independently selected halogens;
R² is H; C₁-C₆alkyl optionally substituted with from 1 to 3 substituents independently selected from —NH₂, —NH(C₁-C₆alkyl), —N(C₁-C₆alkyl)(C₁-C₆alkyl), C₆-C₁₄aryl, and —C(O)O(C₁-C₆alkyl); —S(O)_{q}—(C₁-C₆alkyl); —S(O)_{q}—(C₁-C₉heteroaryl); —S(O)_{q}—(C₆-C₁₄aryl); or —S(O)_{q}-(4- to 7-membered monocyclic heterocycle group) optionally substituted with from 1 to 3 substituents independently selected from C₁-C₆alkyl and (C₆-C₁₄aryl)alkyl-O—C(O)—;
q is independently 1 or 2;
R⁷ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₂-C₁₀alkenyl; C₂-C₁₀alkynyl; halo; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl; or CHO;
R⁸ and R⁹ are each independently H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
or R⁸ and R⁹, when taken together with the nitrogen to which they are attached, can form a 3- to 7-membered nitrogen containing heterocycle wherein up to two of the carbon atoms of the heterocycle can be replaced with —N(R¹³)—, —O—, S, S(O), or —S(O)₂;
R¹³ is hydrogen or C₁-C₆alkyl;
R¹⁰ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
R¹¹ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
R¹² is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl.

In one aspect, the invention provides compounds of the Formula II:

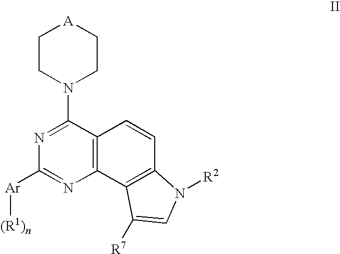

or pharmaceutically acceptable salt thereof, wherein
A is —O—, —CH₂O—, or —S(O)ₘ—;
m is 0, 1, or 2;
Ar is phenyl, naphthyl, or nitrogen-containing mono- or bicyclic C₁-C₉heteroaryl;
R¹ is independently NR³R⁴; NHC(O)NR³R⁴; —NHC(O)OR⁵; R⁵C(O)NH—; R⁵C(O)—; R⁵S(O)ₚNH—; CHO; C₁-C₆hydroxylalkyl-; C₃-C₆hydroxylalkenyl-; (C₆-C₁₄aryl)alkyl optionally substituted by hydroxyl; (C₆-C₁₄aryl)alkyl-O—; (C₁-C₆alkoxy)carbonyl; HO₂C—; R³R⁴NC(O)—; N≡C—; carboxyamido(C₁-C₆alkyl-; hydroxyl; halo; C₁-C₆alkoxy optionally substituted with from 1 to 3 substituents independently selected from C₁-C₆alkoxy, —NH₂, —NH(C₁-C₆alkyl), and —N(C₁-C₆alkyl)(C₁-C₆alkyl); —NH(SO₂)NH—(C₁-C₆alkyl); —NH(SO₂)N—(C₁-C₆alkyl)(C₁-C₆alkyl); —O-heterocycle optionally substituted by C₁-C₆alkyl; H₂NC₁-C₆alkyleneSO₂—; (C₁-C₆alkyl)NHC₁-C₆alkyleneSO₂—_{;} (C₁-C₆alkyl)(C₁-C₆alkyl)NC₁-C₆alkyleneSO₂—; heterocyclyl(C₁-C₆alkyl)SO₂—; carboxyamido(C₁-C₆)alkyl-C(O)—; heterocycle-C(O)—C₁-C₆alkylene-C(O)—; R³R⁴NSO₂C₁-C₆alkylene-C(O)—; or —SO₂NR³R⁴;
n is 0, 1, 2, 3, 4, or 5;
each p is independently 1 or 2;
R³ and R⁴ are each independently:
   (a). H;
   (b). C₁-C₆alkyl optionally substituted with from 1 to 3 substituents independently selected from:
      (i). —NH₂,
      (ii). —NH(C₁-C₆alkyl),
      (iii). —N(C₁-C₆alkyl)(C₁-C₆alkyl),
      (iv). C₁-C₆alkoxy,
      (v). C₃-C₈cycloalkyl,
      (vi). C₃-C₈cycloalkenyl,
      (vii). halo,
      (viii). and C₁-C₉heteroaryl;
   (c). C₁-C₉heteroaryl optionally substituted with from 1 to 3 substituents independently selected from:
      (i). C₁-C₆alkyl,
      (ii). C₁-C₆aminoalkyl-,
      (iii). C₁-C₆hydroxylalkyl-,
      (iv). and C₁-C₉heterocyclyl-;
   (d). heterocyclyl(C₁-C₆alkyl)-;
   (e). (C₁-C₉heterocyclyl)-;
   (f). (C₁-C₉heterocyclyl)-SO₂—;
   (g). C₆-C₁₄aryl optionally substituted with from 1 to 3 substituents independently selected from:
      (i). C₁-C₆alkyl,
      (ii). C₁-C₆alkoxy,
      (iii). C₁-C₆aminoalkyl-,
      (iv). C₁-C₆hydroxylalkyl-,
      (v). C₁-C₆aminoalkyl-NH—,
      (vi). C₁-C₆hydroxylalkyl-NH—,
      (vii). halo,
      (viii). C₁-C₉heterocyclyl,
      (ix). (C₁-C₉heteroaryl)-O—,
      (x). —(C₁-C₉heterocycle)-O—,
      (xi). (C₁-C₉heterocyclyl)-S—,
      (xii). (C₁-C₉heterocyclyl)-CO—,
      (xiii). (C₁-C₆alkyl)-NH—C(O)—,
      (xiv). (C₁-C₆alkyl)(C₁-C₆alkyl)N—C(O)—,
      (xv). H₂NNH—C(O)—,
      (xvi). R⁵—C(O)—,
      (xvii). (C₁-C₆alkyl)-NH—NH—C(O)—,
      (xviii). (C₁-C₆alkyl)(C₁-C₆alkyl)NNH—C(O)—,
      (xix). (C₁-C₉heteroaryl)NH—C(O)—,
      (xx). (C₆-C₁₄aryl)NH—C(O)—,
      (xxi). (C₁-C₆alkyl)-SO₂—,
      (xxii). (C₁-C₉heterocyclyl)-SO₂—,
      (xxiii). H₂NS(O)₂—,
      (xxiv). (C₁-C₆alkyl)NH—SO₂—,
      (xxv). (C₁-C₆alkyl)(C₁-C₆alkyl)N—SO₂—,
      (xxvi). H₂NNHS(O)₂—,
      (xxvii). (C₁-C₆alkyl)NH—NH—SO₂—,
      (xxviii). (C₁-C₆alkyl)(C₁-C₆alkyl)N—NH—SO₂—,
      (xxix). (C₁-C₉heteroaryl)NH—S(O)₂—,
      (xxx). (C₆-C₁₄aryl)NH—S(O)₂—,
      (xxxi). and perfluoro(C₁-C₆)alkyl-;
   (h). or C₃-C₈cycloalkyl-;
or R³ and R⁴, when taken together with the nitrogen to which they are attached, can form a 3- to 7-membered nitrogen containing heterocycle wherein up to two of the carbon atoms of the heterocycle can be replaced with —N(R⁶)—, —O—, S, S(O), or —S(O)₂—;
R⁶ is hydrogen or C₁-C₆alkyl;
R⁵ is C₁-C₆alkyl-, C₃-C₈cycloalkyl-, C₁-C₉heteroaryl, or C₆-C₁₄aryl- optionally substituted with from 1 to 3 substituents independently selected halogens;
R² is H; C₁-C₆alkyl optionally substituted with from 1 to 3 substituents independently selected from —NH₂, —NH(C₁-C₆alkyl), —N(C₁-C₆alkyl)(C₁-C₆alkyl), C₆-C₁₄aryl, and —C(O)O(C₁-C₆alkyl); —S(O)_{q}—(C₁-C₆alkyl); —S(O)_{q}—(C₁-C₉heteroaryl); —S(O)_{q}—(C₆-C₁₄aryl); or —S(O)_{q}—(4- to 7-membered monocyclic heterocycle group) optionally substituted with from 1 to 3 substituents independently selected from C₁-C₆alkyl and (C₆-C₁₄aryl)alkyl-O—C(O)—;
q is independently 1 or 2;
R⁷ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₂-C₁₀alkenyl; C₂-C₁₀alkynyl; halo; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl; or CHO;

In one aspect, R¹ is independently NR³R⁴; NHC(O)NR³R⁴; —NHC(O)OR⁵; R⁵C(O)NH—; R⁵S(O)ₚNH—; CHO; C₁-C₆hydroxylalkyl-; C₃-C₆hydroxylalkenyl-; (C₆-C₁₄aryl)alkyl optionally substituted by hydroxyl; (C₆-C₁₄aryl)alkyl-O—; (C₁-C₆alkoxy)carbonyl; HO₂C—; R³R⁴NC(O)—; N≡C—; carboxyamido(C₁-C₆)alkyl-; hydroxyl; halo; C₁-C₆alkoxy optionally substituted with from 1 to 3 substituents independently selected from C₁-C₆alkoxy, —NH₂, —NH(C₁-C₆alkyl), and —N(C₁-C₆alkyl)(C₁-C₆alkyl); —NH(SO₂)NH—(C₁-C₆alkyl); —O-heterocycle optionally substituted by C₁-C₆alkyl; H₂NC₁-C₆alkyleneSO₂—; (C₁-C₆alkyl)NHC₁-C₆alkyleneSO₂—_{;} (C₁-C₆alkyl)(C₁-C₆alkyl)NC₁-C₆alkyleneSO₂—; heterocyclyl(C₁-C₆alkyl)SO₂—; carboxyamido(C₁-C₆)alkyl-C(O)—; heterocycle-C(O)—C₁-C₆alkylene-C(O)—; R³R⁴NSO₂C₁-C₆alkylene-C(O)—; or —SO₂NR³R⁴;

In one aspect, R³ and R⁴ are each independently H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl optionally substituted with from 1 to 3 substituents independently selected from C₁-C₆alkyl, C₁-C₆aminoalkyl-, C₁-C₆hydroxylalkyl- and C₁-C₉heterocyclyl; C₆-C₁₄aryl optionally substituted with from 1 to 3 substituents independently selected from C₁-C₆alkyl, C₁-C₆alkoxy, C₁-C₆aminoalkyl-, C₁-C₆hydroxylalkyl-, C₁-C₆aminoalkyl-NH—, C₁-C₆hydroxylalkyl-NH—, halo, C₁-C₉heterocyclyl, (C₁-C₉heteroaryl)-O—, —(C₁-C₉heterocycle)-O—, (C₁-C₉heterocyclyl)-S—, (C₁-C₉heterocyclyl)-CO—, (C₁-C₆alkyl)-NH—C(O)—, (C₁-C₆alkyl)(C₁-C₆alkyl)N—C(O)—, H₂NNH—C(O)—, (C₁-C₆alkyl)-NH—NH—C(O)—, (C₁-C₆alkyl)(C₁-C₆alkyl)NNH—C(O)—(C₁-C₉heteroaryl)NH—C(O)—, (C₆-C₁₄aryl)NH—C(O)—, (C₁-C₆alkyl)-SO₂—, (C₁-C₉heterocyclyl)-SO₂—, H₂NS(O)₂—, (C₁-C₆alkyl)NH—SO₂—, (C₁-C₆alkyl)(C₁-C₆alkyl)N—SO₂—, H₂NNHS(O)₂—, (C₁-C₆alkyl)NH—NH—SO₂—, (C₁-C₆alkyl)(C₁-C₆alkyl)N—NH—SO₂—, (C₁-C₉heteroaryl)NH—S(O)₂—, (C₆-C₁₄aryl)NH—S(O)₂—, and perfluoro(C₁-C₆)alkyl; or C₃-C₈cycloalkyl;

In one aspect, R⁵ is C₁-C₆alkyl or C₆-C₁₄aryl;

In one aspect, n is 1.

In one aspect, A is —O—.

In one aspect, R¹ is hydroxyl.

In one aspect, R¹ is NH₂.

In one aspect, R¹ is —NHC(O)NR³R⁴.

In one aspect, R³ is C₁-C₆alkyl, C₁-C₉heteroaryl, or C₆-C₁₄aryl.

In one aspect, R³ is methyl or 4-pyridyl.

In one aspect, R³ is methyl.

In one aspect, R³ is propyl.

In one aspect, R³ is phenyl.

In one aspect, R³ is 4-pyridyl.

In one aspect, R² is C₁-C₆alkyl optionally substituted with —N(C₁-C₆alkyl)(C₁-C₆alkyl); —S(O)_{q}—(C₁-C₆alkyl); or —S(O)_{q}—(C₆-C₁₄aryl).

In one aspect, R² is C₁-C₆alkyl or S(O)_{q}—(C₁-C₆alkyl).

In one aspect, R² is methyl or —SO₂—CH₃.

In one aspect, R² is methyl.

In one aspect, R² is —S(O)_{q}—(C₁-C₆alkyl); —S(O)_{q}—(C₁-C₉heteroaryl); —S(O)_{q}—(C₆-C₁₄aryl); —S(O)_{q}-(4- to 7-membered monocyclic heterocycle group) optionally substituted with from 1 to 3 substituents independently selected from C₁-C₆alkyl or (C₆-C₁₄aryl)alkyl-O—C(O)—.

In one aspect, R² is —SO₂—CH₃.

In one aspect, R² is —SO₂—C₆H₅.

In one aspect, R⁷ is H.

In one aspect, A is —O—, Ar is phenyl, n is 1, R¹ is —NHC(O)NR³R⁴, R³ is 4-pyridyl, R⁴ is H, R² is methyl, and R⁷ is H.

In one aspect, A is —O—, Ar is 4-pyrimidinyl, n is 1, R¹ is 4-NH₂, R² is —SO₂—C₆H₅, and R⁷ is H.

In one aspect, A is —O—, Ar is phenyl, n is 1, R¹ is meta-hydroxyl, R² is H, and R⁷ is CHO.

The following compounds exemplify illustrative compounds of Formula II:
4-(7-methyl-4-morpholin-4-yl-7H-pyrrolo[2,3-h]quinazolin-2-yl)aniline;
1-methyl-3-[4-(7-methyl-4-morpholin-4-yl-7H-pyrrolo[2,3-h]quinazolin-2-yl)phenyl]urea;
1-ethyl-3-[4-(7-methyl-4-morpholin-4-yl-7H-pyrrolo[2,3-h]quinazolin-2-yl)phenyl]urea;
1-[2-(dimethylamino)ethyl]-3-[4-(7-methyl-4-morpholin-4-yl-7H-pyrrolo[2,3-h]quinazolin-2-yl)phenyl]urea;
1-[3-(dimethylamino)propyl]-3-[4-(7-methyl-4-morpholin-4-yl-7H-pyrrolo[2,3-h]quinazolin-2-yl)phenyl]urea;
4-methyl-N-[4-(7-methyl-4-morpholin-4-yl-7H-pyrrolo[2,3-h]quinazolin-2-yl)phenyl]piperazine-1-carboxamide;
1-(2-furylmethyl)-3-[4-(7-methyl-4-morpholin-4-yl-7H-pyrrolo[2,3-h]quinazolin-2-yl)phenyl]urea;
1-[3-(1H-imidazol-1-yl)propyl]-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-pyridin-2-ylurea;
1-[4-(7-methyl-4-morpholin-4-yl-7H-pyrrolo[2,3-h]quinazolin-2-yl)phenyl]-3-phenylurea;
1-[4-(7-methyl-4-morpholin-4-yl-7H-pyrrolo[2,3-h]quinazolin-2-yl)phenyl]-3-pyridin-4-ylurea;
1-(4-isopropylphenyl)-3-[4-(7-methyl-4-morpholin-4-yl-7H-pyrrolo[2,3-h]quinazolin-2-yl)phenyl]urea;
1-(3-chlorophenyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-[4-(trifluoromethyl)phenyl]urea;
1-[4-(7-methyl-4-morpholin-4-yl-7H-pyrrolo[2,3-h]quinazolin-2-yl)phenyl]-3-(pyridin-2-ylmethyl)urea;
N-[4-(7-methyl-4-morpholin-4-yl-7H-pyrrolo[2,3-h]quinazolin-2-yl)phenyl]acetamide;
N-[4-(7-methyl-4-morpholin-4-yl-7H-pyrrolo[2,3-h]quinazolin-2-Myl)phenyl]nicotinamide;
N-[4-(7-methyl-4-morpholin-4-yl-7H-pyrrolo[2,3-h]quinazolin-2-yl)phenyl]isonicotinamide;
4-fluoro-N-[4-(7-methyl-4-morpholin-4-yl-7H-pyrrolo[2,3-h]quinazolin-2-yl)phenyl]benzamide;
ethyl [4-(7-methyl-4-morpholin-4-yl-7H-pyrrolo[2,3-h]quinazolin-2-yl)phenyl]carbamate;
N-[4-(7-methyl-4-morpholin-4-yl-7H-pyrrolo[2,3-h]quinazolin-2-yl)phenyl]methanesulfonamide;
N-[4-(7-methyl-4-morpholin-4-yl-7H-pyrrolo[2,3-h]quinazolin-2-yl)phenyl]benzenesulfonamide;
3-(7-methyl-4-morpholin-4-yl-7H-pyrrolo[2,3-h]quinazolin-2-yl)benzaldehyde;
1-[3-(7-methyl-4-morpholin-4-yl-7H-pyrrolo[2,3-h]quinazolin-2-yl)phenyl]ethanol;
1-[3-(7-methyl-4-morpholin-4-yl-7H-pyrrolo[2,3-h]quinazolin-2-yl)phenyl]propan-1-ol;
1-[3-(7-methyl-4-morpholin-4-yl-7H-pyrrolo[2,3-h]quinazolin-2-yl)phenyl]prop-2-en-1-ol;
1-[3-(7-methyl-4-morpholin-4-yl-7H-pyrrolo[2,3-h]quinazolin-2-yl)phenyl]but-3-en-1-ol;
3-methyl-1-[3-(7-methyl-4-morpholin-4-yl-7H-pyrrolo[2,3-h]quinazolin-2-yl)phenyl]butan-1-ol;
[3-(7-methyl-4-morpholin-4-yl-7H-pyrrolo[2,3-h]quinazolin-2-yl)phenyl](phenyl)methanol;
(3-{7-[2-(dimethylamino)ethyl]-4-morpholin-4-yl-7H-pyrrolo[2,3-h]quinazolin-2-yl}phenyl)methanol;
2-{2-[3-(benzyloxy)phenyl]-4-morpholin-4-yl-7H-pyrrolo[2,3-h]quinazolin-7-yl}-N,N-dimethylethanamine;
3-{7-[2-(dimethylamino)ethyl]-4-morpholin-4-yl-7H-pyrrolo[2,3-h]quinazolin-2-yl}phenol;
1-(4-{7-[2-(dimethylamino)ethyl]-4-morpholin-4-yl-7H-pyrrolo[2,3-h]quinazolin-2-yl}phenyl)-3-pyridin-4-ylurea;
5-{7-[2-(dimethylamino)ethyl]-4-morpholin-4-yl-7H-pyrrolo[2,3-h]quinazolin-2-yl}pyrimidin-2-amine;
3-(7-methyl-4-morpholin-4-yl-7H-pyrrolo[2,3-h]quinazolin-2-yl)phenol;
Methyl 4-(7-methyl-4-morpholin-4-yl-7H-pyrrolo[2,3-h]quinazolin-2-yl)benzoate;
3-(7-methyl-4-morpholin-4-yl-7H-pyrrolo[2,3-h]quinazolin-2-yl)phenyl]methanol;
4-(7-methyl-4-morpholin-4-yl-7H-pyrrolo[2,3-h]quinazolin-2-yl)benzoic acid;
3-(7-methyl-4-morpholin-4-yl-7H-pyrrolo[2,3-h]quinazolin-2-yl)benzamide;
3-(7-methyl-4-morpholin-4-yl-7H-pyrrolo[2,3-h]quinazolin-2-yl)benzonitrile;
3-(7-methyl-4-morpholin-4-yl-7H-pyrrolo[2,3-h]quinazolin-2-yl)aniline;
5-(7-methyl-4-morpholin-4-yl-7H-pyrrolo[2,3-h]quinazolin-2-yl)pyridin-2-amine;
5-(7-methyl-4-morpholin-4-yl-7H-pyrrolo[2,3-h]quinazolin-2-yl)pyrimidin-2-amine;
N-[3-(7-methyl-4-morpholin-4-yl-7H-pyrrolo[2,3-h]quinazolin-2-yl)benzyl]acetamide;
2-(1H-indol-4-yl)-7-methyl-4-morpholin-4-yl-7H-pyrrolo[2,3-h]quinazoline;
3-(7-benzyl-4-morpholin-4-yl-7H-pyrrolo[2,3-h]quinazolin-2-yl)phenol;
2-(6-methoxypyridin-3-yl)-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyridin-2-ol;
2-(1H-indol-4-yl)-7-methyl-4-morpholin-5-yl-7H-pyrrolo[2,3-h]quinazoline;
2-(2-methoxypyrimidin-5-yl)-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyrimidin-2-ol;
2-(3-fluorophenyl)-7-methyl-4-morpholin-4-yl-7H-pyrrolo[2,3-h]quinazoline;
4-chloro-3-(7-methyl-4-morpholin-4-yl-7H-pyrrolo[2,3-h]quinazolin-2-yl)phenol;
1-[4-(7-methyl-4-morpholin-4-yl-7H-pyrrolo[2,3-h]quinazolin-2-yl)phenyl]-3-propylurea;
N,N-dimethyl-N′-[3-(7-methyl-4-morpholin-4-yl-7H-pyrrolo[2,3-h]quinazolin-2-yl)phenyl]sulfamide;
N-cyclopropyl-3-(7-methyl-4-morpholin-4-yl-7H-pyrrolo[2,3-h]quinazolin-2-yl)benzenesulfonamide;
3-(4-morpholin-4-yl-7H-pyrrolo[2,3-h]quinazolin-2-yl)phenyl]methanol;
-(4-morpholin-4-yl-7H-pyrrolo[2,3-h]quinazolin-2-yl)pyrimidin-2-amine;
3-[7-(methylsulfonyl)-4-morpholin-4-yl-7H-pyrrolo[2,3-h]quinazolin-2-yl]phenol;
tert-butyl [2-(6-aminopyridin-3-yl)-4-morpholin-4-yl-7H-pyrrolo[2,3-h]quinazolin-7-yl]acetate;

benzyl 4-[(4-morpholin-4-yl-2-{4-[(pyridin-4-ylcarbamoyl)amino]phenyl}-7H-pyrrolo[2,3-h]quinazolin-7-yl)sulfonyl]piperidine-1-carboxylate;
1-{4-[4-morpholin-4-yl-7-(piperidin-4-ylsulfonyl)-7H-pyrrolo[2,3-h]quinazolin-2-yl]phenyl}-3-pyridin-4-ylurea;
1-(4-{7-[(1-methylpiperidin-4-yl)sulfonyl]-4-morpholin-4-yl-7H-pyrrolo[2,3-h]quinazolin-2-yl}phenyl)-3-pyridin-4-ylurea;
1-(4-{7-[(1-ethylpiperidin-4-yl)sulfonyl]-4-morpholin-4-yl-7H-pyrrolo[2,3-h]quinazolin-2-yl}phenyl)-3-pyridin-4-ylurea;
1-(4-{7-[(1-isopropylpiperidin-4-yl)sulfonyl]-4-morpholin-4-yl-7H-pyrrolo[2,3-h]quinazolin-2-yl}phenyl)-3-pyridin-4-ylurea;
benzyl 4-{[2-(3-hydroxyphenyl)-4-morpholin-4-yl-7H-pyrrolo[2,3-h]quinazolin-7-yl]sulfonyl}piperidine-1-carboxylate;
3-[4-morpholin-4-yl-7-(piperidin-4-ylsulfonyl)-7H-pyrrolo[2,3-h]quinazolin-2-yl]phenol;
2-[3-(benzyloxy)phenyl]-4-morpholin-4-yl-7-(phenylsulfonyl)-7H-pyrrolo[2,3-h]quinazoline;
3-[4-morpholin-4-yl-7-(phenylsulfonyl)-7H-pyrrolo[2,3-h]quinazolin-2-yl]phenol;
{3-[4-morpholin-4-yl-7-(phenylsulfonyl)-7H-pyrrolo[2,3-h]quinazolin-2-yl]phenyl}methanol;
5-[4-morpholin-4-yl-7-(phenylsulfonyl)-7H-pyrrolo[2,3-h]quinazolin-2-yl]pyrimidin-2-amine;
2-(1H-indazol-4-yl)-4-morpholin-4-yl-7-(phenylsulfonyl)-7H-pyrrolo[2,3-h]quinazoline;
5-[7-(methylsulfonyl)-4-morpholin-4-yl-7H-pyrrolo[2,3-h]quinazolin-2-yl]pyrimidin-2-amine;
{3-[7-(methylsulfonyl)-4-morpholin-4-yl-7H-pyrrolo[2,3-h]quinazolin-2-yl]phenyl}methanol;
2-[5-(methoxymethoxy)pyridin-3-yl]-7-(methylsulfonyl)-4-morpholin-4-yl-7H-pyrrolo[2,3-h]quinazoline;
5-[7-(methylsulfonyl)-4-morpholin-4-yl-7H-pyrrolo[2,3-h]quinazolin-2-yl]pyridin-3-ol;
2-[5-(methoxymethoxy)pyridin-3-yl]-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyridin-3-ol;
2-(1H-indazol-4-yl)-7-methyl-4-morpholin-4-yl-7H-pyrrolo[2,3-h]quinazoline;
2-(1H-indazol-4-yl)-4-morpholin-4-yl-7H-pyrrolo[2,3-h]quinazoline;
2-[3-(benzyloxy)phenyl]-4-morpholin-4-yl-7H-pyrrolo[2,3-h]quinazoline;
2-(3-hydroxyphenyl)-4-morpholin-4-yl-7H-pyrrolo[2,3-h]quinazoline-9-carbaldehyde;
[3-(7-benzyl-4-morpholin-4-yl-7H-pyrrolo[2,3-h]quinazolin-2-yl)phenyl]methanol;
1-{4-[7-(methylsulfonyl)-4-morpholin-4-yl-7H-pyrrolo[2,3-h]quinazolin-2-yl]phenyl}-3-phenylurea;
1-{4-[7-(methylsulfonyl)-4-morpholin-4-yl-7H-pyrrolo[2,3-h]quinazolin-2-yl]phenyl}-3-pyridin-3-ylurea;
ethyl {4-[7-(methylsulfonyl)-4-morpholin-4-yl-7H-pyrrolo[2,3-h]quinazolin-2-yl]phenyl}carbamate;
N-{4-[7-(methylsulfonyl)-4-morpholin-4-yl-7H-pyrrolo[2,3-h]quinazolin-2-yl]phenyl}cyclopropanecarboxamide;
N-{4-[7-(methylsulfonyl)-4-morpholin-4-yl-7H-pyrrolo[2,3-h]quinazolin-2-yl]phenyl}butanamide;
1-ethyl-3-{4-[7-(methylsulfonyl)-4-morpholin-4-yl-7H-pyrrolo[2,3-h]quinazolin-2-yl]phenyl}urea;
methyl {4-[7-(methylsulfonyl)-4-morpholin-4-yl-7H-pyrrolo[2,3-h]quinazolin-2-yl]phenyl}carbamate;
N-{4-[7-(methylsulfonyl)-4-morpholin-4-yl-7H-pyrrolo[2,3-h]quinazolin-2-yl]phenyl}propanamide;
N-{4-[7-(methylsulfonyl)-4-morpholin-4-yl-7H-pyrrolo[2,3-h]quinazolin-2-yl]phenyl}acetamide;
ethyl (4-{7-[2-(dimethylamino)ethyl]-4-morpholin-4-yl-7H-pyrrolo[2,3-h]quinazolin-2-yl}phenyl)carbamate;
1-(4-{7-[2-(dimethylamino)ethyl]-4-morpholin-4-yl-7H-pyrrolo[2,3-h]quinazolin-2-yl}phenyl)-3-ethylurea;
1-(4-{7-[2-(dimethylamino)ethyl]-4-morpholin-4-yl-7H-pyrrolo[2,3-h]quinazolin-2-yl}phenyl)-3-phenylurea;
N-(4-{7-[2-(dimethylamino)ethyl]-4-morpholin-4-yl-7H-pyrrolo[2,3-h]quinazolin-2-yl}phenyl)cyclopropanecarboxamide;
N-(4-{7-[2-(dimethylamino)ethyl]-4-morpholin-4-yl-7H-pyrrolo[2,3-h]quinazolin-2-yl}phenyl)butanamide;
methyl (4-{7-[2-(dimethylamino)ethyl]-4-morpholin-4-yl-7H-pyrrolo[2,3-h]quinazolin-2-yl}phenyl)carbamate;
1-(1-methylethyl)-3-[4-(7-methyl-4-morpholin-4-yl-7H-pyrrolo[2,3-h]quinazolin-2-yl)phenyl]urea;
1-(cyclopropylmethyl)-3-[4-(7-methyl-4-morpholin-4-yl-7H-pyrrolo[2,3-h]quinazolin-2-yl)phenyl]urea;
1-(2-methoxyethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(tetrahydrofuran-2-ylmethyl)urea;
1-(2-cyclohex-1-en-1-ylethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(3-pyrrolidin-1-ylpropyl)urea;
1-cyclopentyl-3-[4-(7-methyl-4-morpholin-4-yl-7H-pyrrolo[2,3-h]quinazolin-2-yl)phenyl]urea;
1-cyclobutyl-3-[4-(7-methyl-4-morpholin-4-yl-7H-pyrrolo[2,3-h]quinazolin-2-yl)phenyl]urea;
1-cyclopropyl-3-[4-(7-methyl-4-morpholin-4-yl-7H-pyrrolo[2,3-h]quinazolin-2-yl)phenyl]urea;
1-cyclohexyl-3-[4-(7-methyl-4-morpholin-4-yl-7H-pyrrolo[2,3-h]quinazolin-2-yl)phenyl]urea;
propyl {4-[7-(methylsulfonyl)-4-morpholin-4-yl-7H-pyrrolo[2,3-h]quinazolin-2-yl]phenyl}carbamate;
1-methyl-3-{4-[7-(methylsulfonyl)-4-morpholin-4-yl-7H-pyrrolo[2,3-h]quinazolin-2-yl]phenyl}urea;
1-(4-{7-[2-(dimethylamino)ethyl]-4-morpholin-4-yl-7H-pyrrolo[2,3-h]quinazolin-2-yl}phenyl)-3-pyridin-3-ylurea;
N-(4-{7-[2-(dimethylamino)ethyl]-4-morpholin-4-yl-7H-pyrrolo[2,3-h]quinazolin-2-yl}phenyl)acetamide;
1-(4-{7-[2-(dimethylamino)ethyl]-4-morpholin-4-yl-7H-pyrrolo[2,3-h]quinazolin-2-yl}phenyl)-3-methylurea;
1-(3-acetylphenyl)-3-[4-(7-methyl-4-morpholin-4-yl-7H-pyrrolo[2,3-h]quinazolin-2-yl)phenyl]urea;
1-(4-acetylphenyl)-3-[4-(7-methyl-4-morpholin-4-yl-7H-pyrrolo[2,3-h]quinazolin-2-yl)phenyl]urea;
1-(3,5-dimethylisoxazol-4-yl)-3-[4-(7-methyl-4-morpholin-4-yl-7H-pyrrolo[2,3-h]quinazolin-2-yl)phenyl]urea;
1-(1,1-dioxidotetrahydrothiophen-3-yl)-3-[4-(7-methyl-4-morpholin-4-yl-7H-pyrrolo[2,3-h]quinazolin-2-yl)phenyl]urea;
1-(2-fluoroethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(2,2,2-trifluoroethyl)urea;
1-[4-(7-methyl-4-morpholin-4-yl-7H-pyrrolo[2,3-h]quinazolin-2-yl)phenyl]-3-(2-pyridin-4-ylethyl)urea;
1-{4-[7-(methylsulfonyl)-4-morpholin-4-yl-7H-pyrrolo[2,3-h]quinazolin-2-yl]phenyl}-3-propylurea;
1-[3-(7-methyl-4-morpholin-4-yl-7H-pyrrolo[2,3-h]quinazolin-2-yl)phenyl]-3-pyridin-4-ylurea;
1-[3-(7-methyl-4-morpholin-4-yl-7H-pyrrolo[2,3-h]quinazolin-2-yl)phenyl]-3-pyridin-3-ylurea;
ethyl [3-(7-methyl-4-morpholin-4-yl-7H-pyrrolo[2,3-h]quinazolin-2-yl)phenyl]carbamate;
N-[3-(7-methyl-4-morpholin-4-yl-7H-pyrrolo[2,3-h]quinazolin-2-yl)phenyl]cyclopropanecarboxamide;
N-[3-(7-methyl-4-morpholin-4-yl-7H-pyrrolo[2,3-h]quinazolin-2-yl)phenyl]butanamide;
1-[3-(7-methyl-4-morpholin-4-yl-7H-pyrrolo[2,3-h]quinazolin-2-yl)phenyl]-3-propylurea;
1-ethyl-3-[3-(7-methyl-4-morpholin-4-yl-7H-pyrrolo[2,3-h]quinazolin-2-yl)phenyl]urea;
N-[3-(7-methyl-4-morpholin-4-yl-7H-pyrrolo[2,3-h]quinazolin-2-yl)phenyl]propanamide;
methyl 4-({[4-(7-ethyl-4-morpholin-4-yl-7H-pyrrolo[2,3-h]quinazolin-2-yl)phenyl]carbamoyl}amino)benzoate;
1-[4-(7-ethyl-4-morpholin-4-yl-7H-pyrrolo[2,3-h]quinazolin-2-yl)phenyl]-3-{4-[(4-methylpiperazin-1-yl)carbonyl]phenyl}urea;
4-({[4-(7-ethyl-4-morpholin-4-yl-7H-pyrrolo[2,3-h]quinazolin-2-yl)phenyl]carbamoyl}amino)-N-[2-(methylamino)ethyl]benzamide;
N-[2-(dimethylamino)ethyl]-4-({[4-(7-ethyl-4-morpholin-4-yl-7H-pyrrolo[2,3-h]quinazolin-2-yl)phenyl]carbamoyl}amino)-N-methylbenzamide; and
1-(4-{[4-(dimethylamino)piperidin-1-yl]carbonyl}phenyl)-3-[4-(7-ethyl-4-morpholin-4-yl-7H-pyrrolo[2,3-h]quinazolin-2-yl)phenyl]urea

The invention also includes pharmaceutical compositions comprising a 7H-pyrrolo[2,3-h]quinazoline compound and a pharmaceutically acceptable carrier. The invention includes a 7H-pyrrolo[2,3-h]quinazoline compound when provided as a pharmaceutically acceptable prodrug, hydrated salt, such as pharmaceutically acceptable salt, or mixtures thereof.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

An “effective amount” when used in connection a 7H-pyrrolo[2,3-h]quinazoline compound of this invention is an amount effective for inhibiting mTOR or PI3K in a subject.

In another aspect, the invention provides a compound of the Formula III:

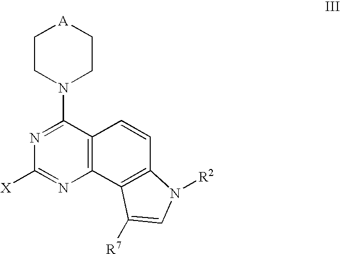

wherein A is —O—, —CH₂O—, or S(O)ₘ;
m is 0, 1, or 2;
X is halogen;
R² is H; C₁-C₆alkyl optionally substituted with from 1 to 3 substituents independently selected from —NH₂, —NH(C₁-C₆alkyl), —N(C₁-C₆alkyl)(C₁-C₆alkyl), C₆-C₁₄aryl, and —C(O)O(C₁-C₆alkyl); —S(O)_{q}—(C₁-C₆alkyl); —S(O)_{q}—(C₁-C₉heteroaryl); —S(O)_{q}—(C₆-C₁₄aryl); or —S(O)_{q}-(4- to 7-membered monocyclic heterocycle group) optionally substituted with from 1 to 3 substituents independently selected from C₁-C₆alkyl and (C₆-C₁₄aryl)alkyl-O—C(O)—;
q is independently 1 or 2;
R⁷ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₂-C₁₀alkenyl; C₂-C₁₀alkynyl; halo; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl; or CHO.

In one aspect, A is —O—.

In one aspect, X is chlorine.

In one aspect, R² is —S(O)_{q}—(C₁-C₆alkyl); —S(O)_{q}—(C₆-C₁₄aryl); or —S(O)_{q}-(4- to 7-membered monocyclic heterocycle group) substituted with (C₆-C₁₄aryl)alkyl-O—C(O)—.

In one aspect, R² is —SO₂CH₃.

In one aspect, R² is benzyl 4-sulfonylpiperidine-1-carboxylate.

In one aspect, R² is —SO₂C₆H₅.

In one aspect, R⁷ is H.

In one aspect, A is —O—, X is Cl, R² is benzyl 4-sulfonylpiperidine-1-carboxylate, and R⁷ is H.

In one aspect, A is —O—, X is Cl, R² is —SO₂C₆H₅, and R⁷ is H.

In one aspect, A is —O—, X is Cl, R² is —SO₂—CH₃, and R⁷ is H.

The following compounds exemplify illustrative compounds of Formula III:
benzyl 4-[(2-chloro-4-morpholin-4-yl-7H-pyrrolo[2,3-h]quinazolin-7-yl)sulfonyl]piperidine-1-carboxylate;
2-chloro-4-morpholin-4-yl-7-(phenylsulfonyl)-7H-pyrrolo[2,3-h]quinazoline;
2-chloro-7-(methylsulfonyl)-4-morpholin-4-yl-7H-pyrrolo[2,3-h]quinazoline;
2-chloro-7-(2-(dimethylamino)ethyl)-4-morpholin-4-yl-7H-pyrrolo[2,3h]quinazoline; and
2-chloro-7-methyl-4-morpholin-4-yl-7H-pyrrolo[2,3-h]quinazoline.

In other aspects, the invention provides a composition comprising a compound of Formula I, a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, lavendustin A, hydroxyzine, glatiramer acetate, interferon beta-1a, interferon beta-1b, natalizumab and lavendustin A; and a pharmaceutically acceptable carrier.

In other aspects, the second compound is Avastin.

In other aspects, the invention provides a method of treating a PI3K-related disorder, comprising administering to a mammal in need thereof a compound of Formula I in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of Formula I in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating a hSMG-1-related disorder, comprising administering to a mammal in need thereof a compound of Formula I in an amount effective to treat a hSMG-1-related disorder.

In other aspects, the hSMG-1-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the hSMG-1-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of Formula I in an amount effective to treat advanced renal cell carcinoma.

In other aspects, the invention provides a method of treating acute lymphoblastic leukemia, comprising administering to a mammal in need thereof a compound of Formula I in an amount effective to treat acute lymphoblastic leukemia.

In other aspects, the invention provides a method of treating acute malignant melanoma, comprising administering to a mammal in need thereof a compound of Formula I in an amount effective to treat malignant melanoma.

In other aspects, the invention provides a method of treating soft-tissue or bone sarcoma, comprising administering to a mammal in need thereof a compound of Formula I in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of Formula I; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier. in an amount effective to treat the cancer.

In other aspects, the invention provides a method of inhibiting mTOR in a subject, comprising administering to a subject in need thereof a compound of Formula I in an amount effective to inhibit mTOR.

In other aspects, the invention provides a method of inhibiting PI3K in a subject, comprising administering to a subject in need thereof a compound of Formula I in an amount effective to inhibit PI3K.

In other aspects, the invention provides a method of inhibiting hSMG-1 in a subject, comprising administering to a subject in need thereof a compound of Formula I in an amount effective to inhibit hSMG-1.

In other aspects, the invention provides a method of inhibiting mTOR, PI3K, and hSMG-1 together in a subject, comprising administering to a subject in need thereof a compound of Formula I in an amount effective to inhibit mTOR, PI3K, and hSMG-1.

In another aspect, the invention provides a method of synthesizing compounds of the Formula II comprising: reacting a boronic acid of the formula Rₙ¹—Ar—B(OH)₂ with the 2-chloro-7H-pyrrolo[2,3-h]quinazoline 24:

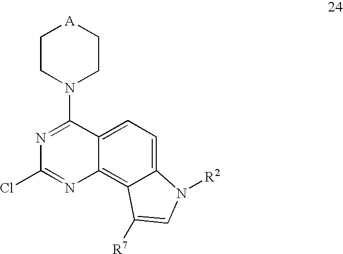

wherein A, Ar, R¹, n, R², and R⁷, are as defined in Formula II:

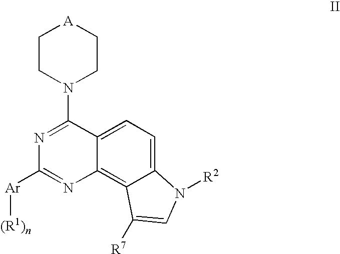

to give the 7H-pyrrolo[2,3-h]quinazoline II.

In one aspect, the invention provides methods of synthesizing compounds of the Formula II further comprising: (a) reacting the 7H-pyrrolo[2,3-h]quinazoline of Formula 17 with an alkylating or acylating agent R²—X to substitute the amino group at position 7 of the 7H-pyrrolo[2,3-h]quinazoline, wherein X is halogen,

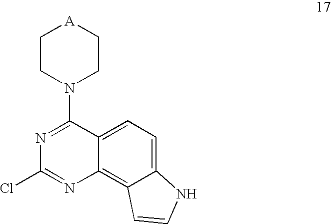

under conditions effective to alkylate or acylate the nitrogen atom at position 7 of the pyrrole ring thereby producing 23,

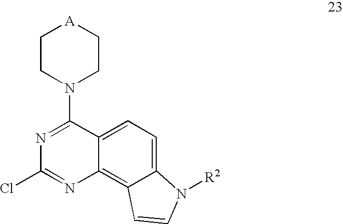

(b) optionally reacting 23 with a formylating agent under Vilsmeier-Haack conditions to formylate the pyrrole ring, thereby producing the chlorinated intermediate 24,

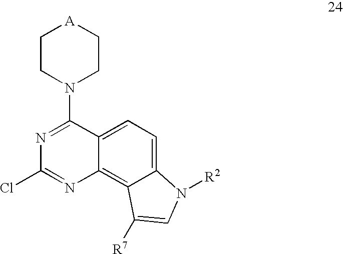

wherein R⁷ is CHO, under conditions effective to replace the hydrogen atom at position 9 of the 7H-pyrrolo[2,3-h]quinazoline. Suitable formylating agents include DMF, N-formylpyrrolidine, N-formylpiperidine, di-isopropylformamide, N0-methyl-N-adamantyl formamide, dicyclohexylformamide, the preformed Vilsmeier reagent ((chloromethylene)dimethylammonium chloride) and any other formylating agent known in the art.

### Definitions

The following definitions are used in connection with the 7H-pyrrolo[2,3-h]quinazoline compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms.

Acyl” refers to from 1 to 8 carbon atoms of a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₈acyl group include acetyl-, benzoyl-, nicotinoyl, propionyl-, isobutyryl-, oxalyl-, and the like. Lower-acyl refers to acyl groups containing one to four carbons. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl.

“Alkenyl” refer to a straight or branched chain unsaturated hydrocarbon containing 2-10 carbon atoms, and containing at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. A C₂-C₁₀alkenyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, and C₃-C₈cycloalkyl.

“Alkoxy” refers to the group R—O— where R is an alkyl group, as defined below. Exemplary C₁-C₆alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl_{,} C₁-C₉heteroaryl, C₃-C₈cycloalkyl, C₁-C₆haloalkyl-, C₁-C₆aminoalkyl-, —OC(O)C₁-C₆alkyl), carboxyamido(C₁-C₆)alkyl-, or —NO₂.

“(Alkoxy)carbonyl-” refers to the group alkyl-O—C(O)—. Exemplary alkoxy groups include but are not limited to (methoxy)carbonyl(acetoxy), (ethoxy)carbonyl(propionoxy), and (t-butoxy)carbonyl (t-butyloxycarbonyl). An (alkoxy)carbonyl- group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl_{,} C₁-C₉heteroaryl, C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂.

“Alkyl” refers to a hydrocarbon chain that may be a straight chain or branched chain, containing the indicated number of carbon atoms. For example, C_{I}-C_{I0} indicates that the group may have from 1 to 10 (inclusive) carbon atoms in it. In the absence of any numerical designation, “alkyl” is a chain (straight or branched) having 1 to 6 (inclusive) carbon atoms in it. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂.

The carbon number as used in the definitions herein refers to carbon backbone and carbon branching, but does not include carbon atoms of the substituents, such as alkoxy substitutions and the like.

“(Alkyl)amido-” refers to a —C(O)NH— group in which the nitrogen atom of said group is attached to a alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an —NH group, the nitrogen atom of said group being attached to a alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂.

“Alkylcarboxy” refers to an alkyl group, defined above, attached to the parent structure through the oxygen atom of a carboxyl (C(O)—O—) functionality. Examples include acetoxy, ethylcarboxy, propylcarboxy, and isopentylcarboxy.

“(Alkyl)carboxyamido-” refers to a —NHC(O)— group in which the carbonyl carbon atom of said group is attached to a alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to the subsets of alkyl, alkenyl and alkynyl groups, as defined above, including the same residues as alkyl, alkenyl, and alkynyl, but having two points of attachment within a chemical structure. Examples of C₁-C₆alkylene include ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to groups of straight chain or branched chain with 1 to 6 carbon atoms, attached to the parent structure through a sulfur atom. Examples include methylthio, ethylthio, n-propylthio, i-propylthio, n-butylthio, i-butylthio, s-butylthio, t-butylthio, n-pentylthio and n-hexylthio.

“Alkynyl” refers to a straight or branched chain unsaturated hydrocarbon containing 2-10 carbon atoms, and containing at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. An alkynyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl)- group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with —NH₂. Representative examples of an C₁-C₆aminoalkyl- group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An aminoalkyl- group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

“Aryl” refers to an aromatic hydrocarbon group containing 6-14 carbon ring atoms. “C₆-C₁₄Aryl” refers to a phenyl, naphthyl, biphenyl, anthryl, tetrahydronaphthyl, fluorenyl, indanyl, biphenylenyl, and acenaphthenyl, groups. Examples of an C₆-C₁₄aryl group include, but are not limited to, phenyl, 1-naphthyl, 2-naphthyl, and 3-biphen-1-yl. An aryl group can be unsubstituted or substituted with one or more of the following groups: C₁-C₆alkyl, halo, haloalkyl-, hydroxyl, hydroxyl(C₁-C₆alkyl)-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to an alkyl group, as defined above, wherein one or more of the alkyl group's hydrogen atoms has been replaced with an C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂.

“(Aryl)amino” refers to a radical of formula (C₆-C₁₄aryl)-NH—, wherein “C₆-C₁₄aryl” is as defined above. Examples of (C₆-C₁₄aryl)amino radicals include, but are not limited to, phenylamino(anilido), 1-naphthlamino, 2-naphthlamino and the like. An (C₆-C₁₄aryl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl.

“(Aryl)oxy” refers to the group Ar—O— where Ar is an C₆-C₁₄aryl group, as defined above. Exemplary (C₆-C₁₄aryl)oxy groups include but are not limited to phenyloxy, α-naphthyloxy, and β-naphthyloxy. A (C₆-C₁₄aryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, -dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Cycloalkyl” refers to a monocyclic, non-aromatic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. A cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Bicyclic cycloalkyl” refers to a bicyclic, non-aromatic, saturated hydrocarbon ring system containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl, haloalkyl-, aminoalkyl-, —OC(O)(C₁-C₆alkyl), carboxyamido(C₁-C₆)alkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carboxyamido(alkyl)-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an C₁-C₆alkylene group as defined above. Exemplary carboxyamido(C₁-C₆)alkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to monocyclic, non-aromatic carbocyclic rings containing 3-10 carbon atoms with one or more carbon-to-carbon double bonds within the ring system. The “cycloalkenyl” may be a single ring or may be multi-ring. Multi-ring structures may be bridged or fused ring structures. A C₃-C₁₀cycloalkenyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl, C₁-C₆haloalkyl-, C₁-C₆aminoalkyl-, —OC(O)(C₁-C₆alkyl), carboxyamido(C₁-C₆)alkyl-, or —NO₂ Additionally, each of any two hydrogen atoms on the same carbon atom of the C₃-C₁₀cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom which has attached to it two alkyl groups, as defined above. Each alkyl group can be independently selected. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ₚ—. R is hydrogen, C₁-C₆alkyl, C₃-C₈cycloalkyl, C₆-C₁₄aryl, C₁-C₉heteroaryl, C₁-C₆aminoalkyl-, or arylamino. Variable p is 0, 1, or 2.

“Halo” or “halogen” is —F, —Cl, —Br or —I.

“Haloalkyl-” refers to a alkyl group, as defined above, wherein one or more of the C₁-C₆alkyl group's hydrogen atoms has been replaced with —F, —Cl, —Br, or —I. Each substitution can be independently selected from —F, —Cl, —Br, or —I. Representative examples of an C₁-C₆haloalkyl- group include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl-” refers to 5-10-membered mono and bicyclic aromatic groups containing at least one heteroatom selected from oxygen, sulfur and nitrogen. Examples of monocyclic C₁-C₉heteroaryl radicals include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Heteroaryl)oxy-” refers to the group Het-O— where Het is a heteroaryl group, as defined above. Exemplary (C₁-C₉heteroaryl)oxy groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

The term “heteroatom” refers to a sulfur, nitrogen, or oxygen atom.

“Heterocycle” or “heterocyclyl-” refers to 3-10-membered mono and bicyclic groups containing at least one heteroatom selected from oxygen, sulfur and nitrogen. A heterocycle may be saturated or partially saturated. Any sulfur atom contained within a heterocycle ring may be at the sulfide (—S—), sulfoxide (—S(O)—), or sulfonyl (—S(O)₂—) oxidation state. Exemplary C₁-C₉heterocycle groups include but are not limited to aziridine, oxirane, oxirene, thiirane, pyrroline, pyrrolidine, dihydrofuran, tetrahydrofuran, dihydrothiophene, tetrahydrothiophene, 1,1-dioxidotetrahydrothiophene, dithiolane, piperidine, decahydroquinoline, tetrahydropyran, pyran, thiane, thiine, piperazine, oxazine, thiazine, dithiane, and dioxane.

“Heterocyclyl(alkyl)” refers to an alkyl group, as defined above, wherein one or more of the alkyl group's hydrogen atoms has been replaced with a heterocycle group as defined above. Heterocyclyl(C₁-C₆alkyl)- moieties include 4-piperidinylmethyl, 1-piperazinylethyl, 4-morpholinylpropyl, 6-piperazinylhexyl, and the like. A heterocyclyl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), 4- to 7-membered monocyclic heterocycle, C₆-C₁₄aryl_{,} C₁-C₉heteroaryl, or C₃-C₈cycloalkyl.

“Hydroxylalkyl-” refers to a alkyl group, as defined above, wherein one or more of the C₁-C₆alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to an alkenyl group, defined above, and substituted on one or more sp³ carbon atoms with a hydroxyl group. Examples of C₃-C₆hydroxylalkenyl- moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

The term “monocyclic heterocycle” refers to a monocyclic 3- to 7-membered aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridinyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A monocyclic heterocycle group can be unsubstituted or substituted with one or more of the following groups: C₁-C₈acyl, C₁-C₆alkyl, heterocyclyl(C₁-C₆alkyl), (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂.

“Bicyclic heterocycle” refers to a bicyclic aromatic, bicyclic cycloalkyl, or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a 6- to 10-membered bicyclic heterocycle group include, but are not limited to, benzimidazolyl, indolyl, indolinyl, isoquinolinyl, indazolyl, quinolinyl, tetrahydroquinolinyl, decahydroquinoline, quinazolinyl, purinyl, benzisoxazolyl, benzoxazolyl, benzthiazolyl, benzodiazolyl, benzotriazolyl, isoindolyl and indazolyl. A bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

“Nitrogen-containing heteroaryl” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing C₁-C₉heteroaryl group can be unsubstituted or substituted with one or more of the following groups: C₁-C₆alkyl, halo, C₁-C₆haloalkyl-, hydroxyl, C₁-C₆hydroxylalkyl-, —NH₂, C₁-C₆aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“Perfluoroalkyl-” refers to alkyl group, defined above, having two or more fluorine atoms. Examples of a C₁-C₆perfluoroalkyl-group include CF₃, CH₂CF₃, CF₂CF₃ and CH(CF₃)₂.

The term “optionally substituted” as used herein means that at least one hydrogen atom of the optionally substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl_{,} C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The 7H-pyrrolo[2,3-h]quinazoline compounds of the present invention exhibit an mTOR inhibitory activity and therefore, can be utilized in order to inhibit abnormal cell growth in which mTOR plays a role. Thus, the 7H-pyrrolo[2,3-h]quinazoline compounds are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the 7H-pyrrolo[2,3-h]quinazoline compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington: The Science and Practice of Pharmacy, 20th Edition. Baltimore, Md.: Lippincott Williams & Wilkins, 2000.

The dosage of the compounds of Formula I to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition as described herein is an amount of active ingredient from about 0.01 mg/kg to 10 mg/kg body weight. For parenteral administration, the dose may be in the range of 0.1 mg/kg to 1 mg/kg body weight for intravenous administration. For oral administration, the dose may be in the range about 0.1 mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

Methods useful for making the 7H-pyrrolo[2,3-h]quinazoline compounds are set forth in the Examples below and generalized in Schemes 1-6:

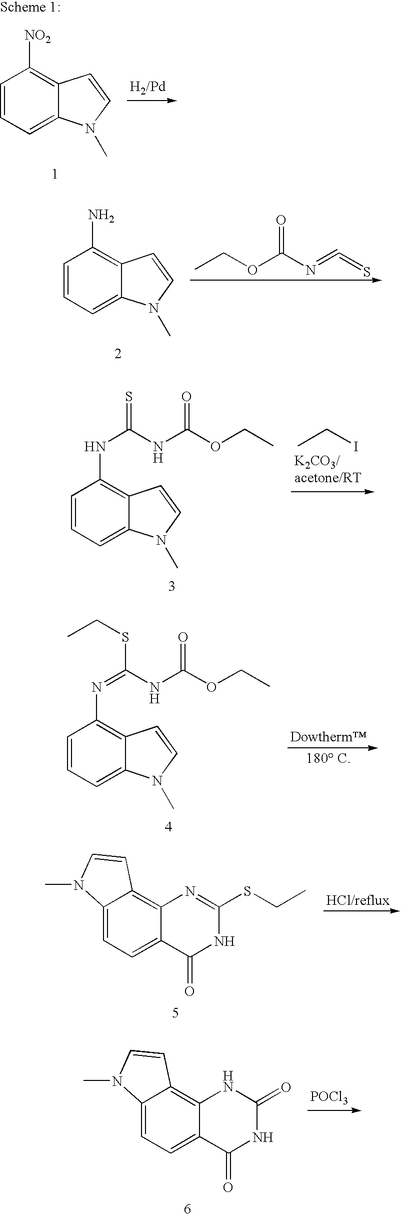

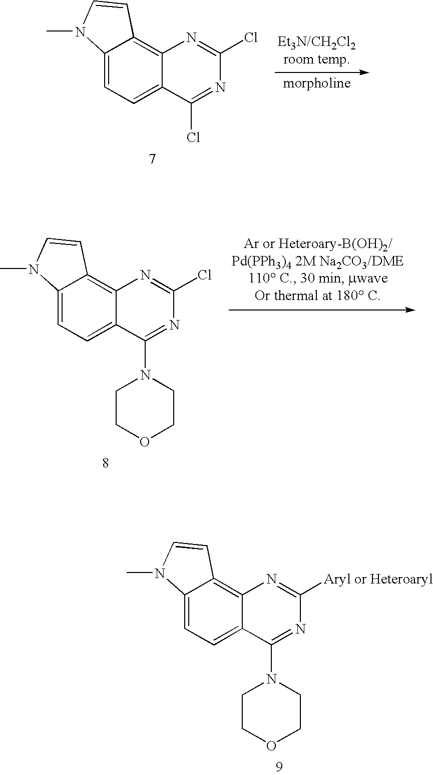

Scheme 1 shows an 8-step synthesis of 4-(7-methyl-2-aryl-7H-pyrrolo[2,3-h]quinazolin-4-yl)morpholine 9 from commercially available 1-methyl-4-nitro-1H-indole. The key and ultimate step is a Suzuki Coupling, where the boronic acid is activated by base. The synthesis is applicable to a wide variety of boronic acids.

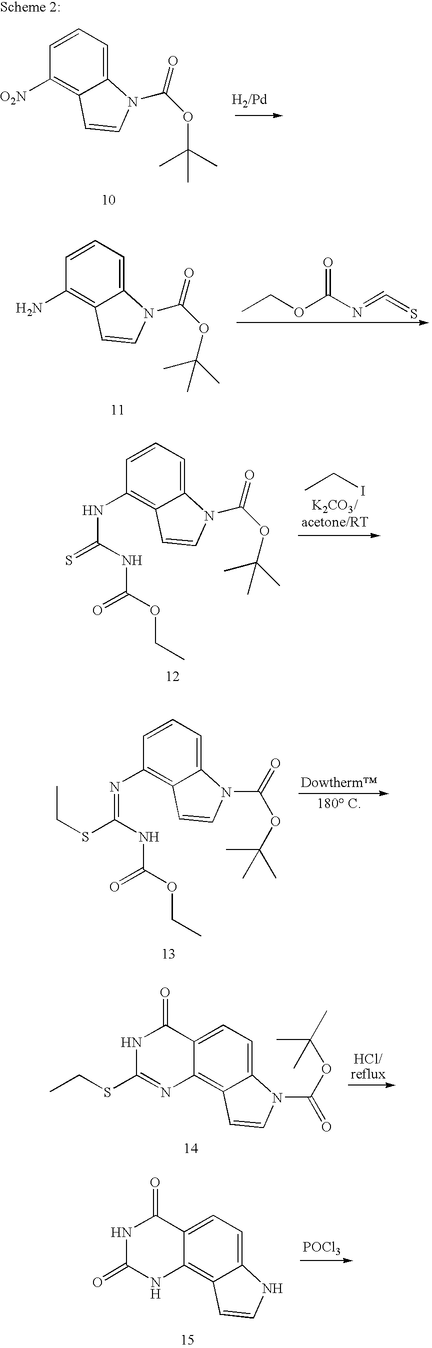

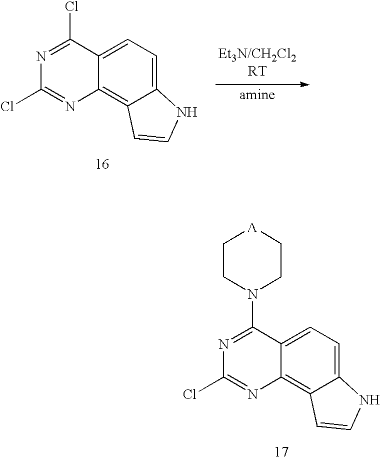

Scheme 2 shows a seven-step synthesis of the key intermediate 2-chloro-4-(cyclic amino)-7H-pyrrolo[2,3-h]quinazoline 17 from tert-butyl 4-nitro-1H-indole-1-carboxylate. Reaction of 2,4-dichloro-7H-pyrrolo[2,3-h]quinazoline (16) with a wide variety of secondary amines can be performed.

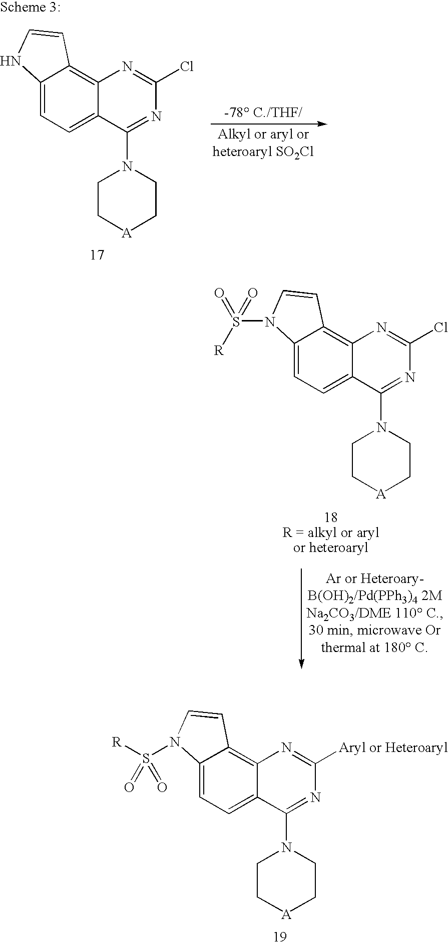

A Suzuki coupling on a variety of 4-(7-(sulfonyl)-2-chloro-7H-pyrrolo[2,3-h]quinazolin-4-yl)amines 18, which were in turn made from key intermediate 17, is shown in Scheme 3.

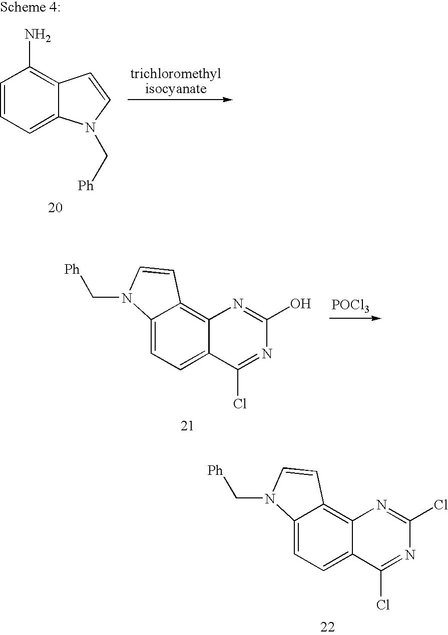

Synthesis of another key intermediate, 7-benzyl-2,4-dichloro-7H-pyrrolo[2,3-h]quinazoline, 22 from 4-amino-1-benzylindole 21 is shown in Scheme 4.

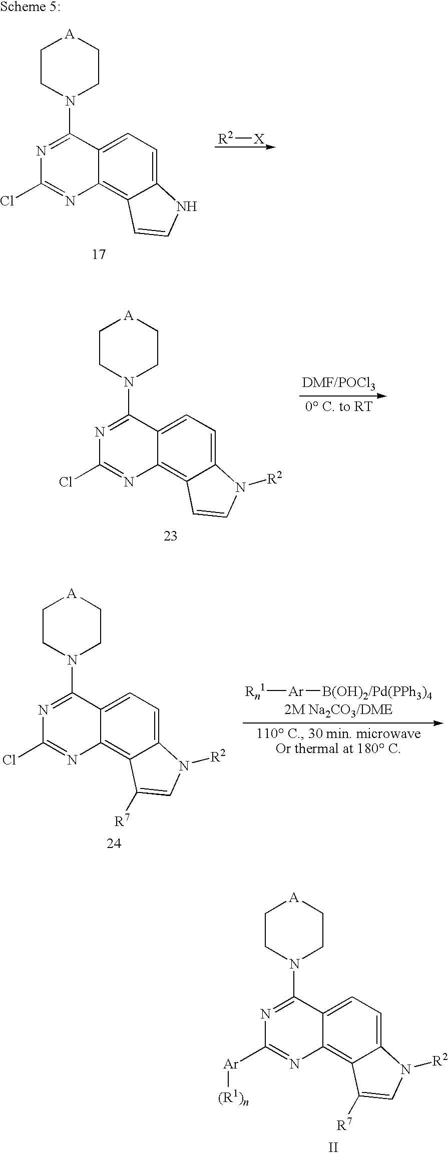

Substitution on positions 7 and 9 of the 7H-pyrrolo[2,3-h]quinazoline ring, starting from key intermediate 17 is shown in Scheme 5.

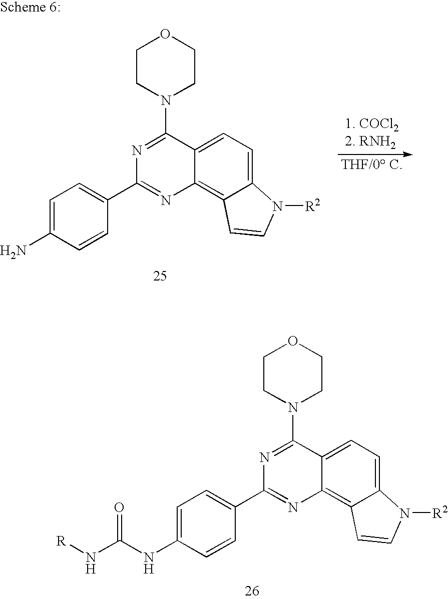

A two-step process adding ureido functionality to position 4 of the benzene ring is shown in Scheme 6.

One of skill in the art will recognize that Schemes 1-6 can be adapted to produce the other 7H-pyrrolo[2,3-h]quinazoline compounds and pharmaceutically acceptable salts of 7H-pyrrolo[2,3-h]quinazoline compounds according to the present invention.

The following abbreviations are used herein and have the indicated definitions: ACN is acetonitrile, AcOH is acetic acid, ATP is adenosine triphosphate, CHAPS is 3[(3-cholamidopropyl)dimethylammonio]-propanesulfonic acid, DEAD is diethyl azodicarboxylate, DIAD is diisopropylazodicarboxylate, DMAP is dimethyl aminopyridine, DMF is N,N-dimethylformamide, DMF-DMA is dimethylformamide dimethyl acetal, DMSO is dimethylsulfoxide. Dowtherm™ is a eutectic mixture of biphenyl (C₁₂H₁₀) and diphenyl oxide (C₁₂H₁₀O). Dowtherm™ is a registered trademark of Dow Corning Corporation. DPBS is Dulbecco's Phosphate Buffered Saline Formulation, EDTA is ethylenediaminetetraacetic acid, ESI stands for Electrospray Ionization, EtOAc is ethyl acetate, EtOH is ethanol, HEPES is 4-(2-hydroxyethyl)-1-piperazineethanesulfonic acid, GMF is Glass, Hunig's Base is diisopropylethylamine, HPLC is high pressure liquid chromatography, LPS is lipopolysaccharide, MeCN is acetonitrile, MeOH is methanol, MS is mass spectrometry, NEt₃ is triethylamine, NMR is nuclear magnetic resonance, PBS is phosphate-buffered saline (pH 7.4), RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, TLC is thin-layer chromatography, and TRIS is tris(hydroxymethyl)aminomethane.

### EXAMPLES

### Methods

The following methods outline the synthesis of the 7H-pyrrolo[2,3-h]quinazoline compounds.

Experimental for the preparation of intermediate 2-chloro-4-morpholin-4-yl-7H-pyrrolo[2,3-h]quinazoline) (Procedure).

### Step 1: Preparation of 1-Boc-4-nitroindole Formula 10

To a stirred solution of 4-nitroindole (4.0 g, 24.6 mmol) in CH₂Cl₂ (50 mL) were added catalytic amount of DMAP (Catalyst) and Boc₂O (5.9 g, 27.1 mmol) at 0° C., and the resulting reaction mixture was stirred at room temperature for additional 3 h. The mixture was diluted with CH₂Cl₂, and washed with water. The organic phase was dried over MgSO₄. The solvent was removed under reduced pressure to give 1-Boc-4-nitroindole as white solid (6.14 g, 95% yield). MS (ESI) m/z 262.1

### Step 2: Preparation of 1-Boc-4-aminoindole Formula 11

To a stirred solution of 1-Boc-4-nitroindole (6.14 g, 23.4 mmol) in EtOH (100 mL) was added 10% Pd/C (614 mg) under N₂. The resulting mixture was shaken under hydrogen (H₂, 50 psi) at room temperature for 8 h. The mixture was filtered through a pad of Celite™, and washed with EtOH. The filtrate was concentrated under reduced pressure to give the product 1-Boc-4-aminoindole as off-white solid (5.23 g, 96% yield). MS (ESI) m/z 233.2

### Step 3: Preparation of 1-Boc-4-(3-(ethoxycarbonyl)thioueido)-indole Formula 12

To a solution of 1-Boc-4-aminoindole (5.23 g, 22.5 mmol) in CH₂Cl₂ was added ethyl isothiocyanatoformate (2.95 g, 22.5 mmol), and the resulting mixture was stirred at room temperature for 2 h. The mixture was concentrated under reduced pressure and triturated with diethyl ether to give the title compound as off-white solid (8.1 g, 99% yield).

### Step 4: Preparation of 1-Boc-4-((ethoxycarbonylamino)ethylthio)methyleneamino)-indole Formula 13

To a stirred solution of 1-Boc-4-(3-(ethoxycarbonyl)thioueido)-indole (3.58 g, 9.85 mmol) in acetone (100 mL) was added K₂CO₃ (2.72 g, 19.7 mmol), followed by addition of iodoethane (1.69 g, 10.8 mmol) at room temperature. The resulting mixture was vigorously stirred at room temperature overnight. The mixture was filtered and washed with acetone. The filtrate was concentrated under reduced pressure, and the residue was treated with CH₂Cl₂ and water. The mixture was extracted with CH₂Cl₂, and the extracts were washed with water, and dried over MgSO₄. The solvent was removed under reduced pressure to provide the title compound as yellow syrup (3.68 g, 95% yield). MS (ESI) m/z 392.2.

### Step 5: Synthesis of 7-Boc-2-(ethylthio)-7H-pyrrolo[2,3h]quinazoline-4-one Formula 14

A mixture of 1-Boc-4-((ethoxycarbonylamino)ethylthio)methyleneamino)-indole (3.68 g, 9.4 mmol) and phenyl ether (70 mL) was heated at 200° C. for 5 h under N₂. The mixture was cooled down to room temperature and diluted with hexanes. The resulting solid was collected by filtration to give the title compound as off-white solid (1.5 g, 46% yield).

### Step 6: Synthesis of 7H-pyrrolo[2,3h]quinazoline-2,4-dione Formula 15

To a solution of 7-Boc-2-(ethylthio)-7H-Pyrrolo[2,3h]quinazoline-4-one (1.5 g, 4.34 mmol) in EtOH (30 mL) was added 6N HCl aqueous solution (30 mL). The resulting mixture was heated at 80° C. overnight, and then cooled down to room temperature, and concentrated under reduced pressure to its half volume. The resulting solid was collected by filtration to give 7H-pyrrolo[2,3h]quinazoline-2,4-dione as off-white solid (780 mg, 89% yield).

### Step 7: Synthesis of 2,4-dichloro-7H-pyrrolo[2,3h]quinazoline Formula 16

A mixture of 7H-pyrrolo[2,3h]quinazoline-2,4-dione (3.0 g, 14.9 mmol) and POCl₃ (60 mL) was heated at 115° C. for 12 h. The mixture was cooled down to room temperature and POCl₃ was distilled off under reduced pressure. The residue was poured onto ice water with stirring. The resulting solid was collected by filtration to give the title compound 2,4-dichloro-7H-pyrrolo[2,3h]quinazoline as off-white solid (2.06 g, 58% yield). MS (ESI) m/z 236.0.

### Step 8: Synthesis of 2-chloro-4-morpholin-4-yl-7H-pyrrolo[2,3h]quinazoline Formula 17 (A=-O—).

To a solution of 2,4-dichloro-7H-pyrrolo[2,3h]quinazoline (474 mg , 2.0 mmol) in CHCl₃ (30 mL) was added morpholine (192 mg, 2.2 mmol), followed by the addition of triethylamine (0.56 mL, 4.0 mmol). The reaction mixture was stirred at room temperature overnight, and quenched with water. Reaction mixture was washed well with water, dried over anhydrous MgSO₄, and filtered. It was concentrated and the separated solid was taken to next step with out purification. The titled compound was obtained total 466 mg, 81% yield. MS (ESI) m/z 289.3.

### Preparation of 2-chloro-7-methyl-4-morpholin-4-yl-7H-pyrrolo[2,3-h]quinazoline Formula 8

### Step 1: Preparation of 1-methyl-4-nitro-1H-indole Formula 1

A mixture of 4-nitro-1H-indole (3.06 g, 18.9 mmole), potassium carbonate anhydrous (13.5 g, 97.8 mmole) and methyl iodide (3.2 g, 22.5 mmole) was heated at reflux in acetone for 8 hours. At the end, reaction mixture was filtered and acetone was evaporated. It was extracted with chloroform (300 mL) and washed water (300 mL). The organic layer was separated, dried with MgSO₄, filtered and concentrated. The yellow solid, (2.20 g, 66% yield) of 1-methyl-4-nitro-1H-indole was used without further purification. (M+H) 177

### Step 2: Preparation of 1-methyl-4-amino-indole Formula 2

An ethanol solution of 1-methyl-4-nitro-1H-indol (2.0 g, 11.36 mmole) was hydrogenated over 10% Pd catalyst at 40 psi for 4 hours. Reaction mixture was filtered through a pad of Celite™ and concentrated. The residue was extracted with chloroform and washed with water. The organic layer was dried with magnesium sulfate, filtered and concentrated to give the amine. (M+H) 147.

### Step 3: Preparation of 1-methyl-4-(3-(ethoxycarbonyl)thioueido)-indole Formula 5

To a solution of 1-methyl-4-aminoindole (1.76 g, 13.3 mmol) in CH₂Cl₂ was added ethyl isothiocyanatoformate (1.75 g, 13.3 mmol), and the resulting mixture was stirred at room temperature for 2 h. The mixture was concentrated under reduced pressure and triturated with diethyl ether to give the title compound as off-white solid (3.5 g, 95% yield). (M+H) 278.

### Step 4: Preparation of 1-methyl-4-((ethoxycarbonylamino)ethylthio)methyleneamino)-indole Formula 4

To a stirred solution of 1-methyl-4-(3-(ethoxycarbonyl)thioueido)-indole (3.4 g, 12.3 mmol) in acetone (300 mL) was added K₂CO₃ (8.4 g, 60.8 mmol), followed by addition of iodoethane (1.98 g, 12.7 mmol) at room temperature. The resulting mixture was vigorously stirred at room temperature overnight. The mixture was filtered and washed with acetone. The filtrate was concentrated under reduced pressure, and the residue was treated with CH₂Cl₂ and water. The mixture was extracted with CH₂Cl₂, and the extracts were washed with water, and dried over MgSO₄. The solvent was removed under reduced pressure to provide the title compound as yellow syrup (3.56 g, 95% yield). (M+H) 306.

### Step 5: Synthesis of 7-methyl-2-(ethylthio)-7H-Pyrrolo[2,3h]quinazoline-4-one Formula 7

A mixture of 1-methyl-4-((ethoxycarbonylamino)ethylthio)methyleneamino)-indole (3.50 g, 11.36 mmol) and phenyl ether (70 mL) was heated at 200° C. for 5 h under N₂. The mixture was cooled down to room temperature and diluted with hexanes. The resulting solid was collected by filtration to give the title compound as off-white solid (2.8 g, 92% yield). (M−H) 257

### Step 6: Synthesis of 7-methyl-pyrrolo[2,3h]quinazoline-2,4-dione Formula 8

To a solution of 7-methyl-2-(ethylthio)-7H-pyrrolo[2,3h]quinazoline-4-one (2.6 g, 410 mmol) in EtOH (100 mL) was added 6N HCl aqueous solution (30 mL). The resulting mixture was heated at 80° C. overnight, and then cooled down to room temperature, and concentrated under reduced pressure to its half volume. The resulting solid was basified with NH₄OH and the separated solid was collected by filtration to give 7-methyl-pyrrolo[2,3h]quinazoline-2,4-dione as off-white solid (1.84 g, 86% yield).

### Step 7: Synthesis of 2,4-dichloro-7-methyl-7H-pyrrolo[2,3h]quinazoline Formula 9

A mixture of 7-methyl-7H-pyrrolo[2,3h]quinazoline-2,4-dione (1.84 g, 8.6 mmol) and POCl₃ (60 mL) was heated at 115° C. for 12 h. The mixture was cooled down to room temperature and POCl₃ was distilled off under reduced pressure. The residue was poured onto ice water with stirring. The resulting solid was collected by filtration to give the title compound 2,4-dichloro-7-methyl-7H-pyrrolo[2,3h]quinazoline as off-white solid (2.0 g, 93% yield). MS (ESI) m/z 252.0.

### Step 8: Synthesis of 7-methyl-2-chloro-4-morpholin-4-yl-7H-pyrrolo[2,3h]quinazoline Formula 8

To a solution of 2,4-dichloro-7-methyl-7H-pyrrolo[2,3h]quinazoline (1.0 g, 3.94 mmol) in CHCl₃ (30 mL) was added morpholine (340 mg, 4.0 mmol), followed by the addition of triethylamine (0.56 mL, 4.0 mmol). The reaction mixture was stirred at room temperature overnight, and quenched with water. Reaction mixture was washed well with water and dried over anhydrous MgSO₄ and filtered. It was concentrated and the separated solid was taken to next step with out purification. The titled compound was obtained as a pale yellow solid. 1.15 g, 96% yield. (M+H) 303.

### Example 1

### Preparation of 4-(7-methyl-4-morpholin-4-yl-7H-pyrrolo[2,3-h]quinazolin-2-yl)aniline

A mixture of 2-chloro-7-methyl-4-morpholin-4-yl-7H-pyrrolo[2,3-h]quinazoline (723 mg, 2.4 mmol), 4-aminophenylboronic acid, pinacol ester (786 mg, 3.6 mmol), Pd(PPh₃)₄ (138 mg, 5 mol %), dimethoxyethane (DME, 8 mL) and 2M Na₂CO₃ (4 mL) was heated at 120° C. for 0.5 h in microwave oven. The reaction mixture was cooled to room temperature, and filtered through a pad of Celite™, washed with THF. The filtrate was concentrated under reduced pressure, and the residue was subjected to flash chromatography in silica gel (EtOAc:Hexanes:CH₂Cl₂=50:30:20) to give the title compound as yellow solid (705 mg, 82% yield). MS (ESI) m/z 360.4.

HRMS: calcd for C₂₁H₂₁N₅O+H⁺, 360.18189; found (ESI-FTMS, [M+H]1+), 360.18254.

### Example 2

### Preparation of 1-methyl-3-[4-(7-methyl-4-morpholin-4-yl-7H-pyrrolo[2,3h]quinazolin-2-yl)phenyl]urea

To a solution of 4-(7-methyl-4-morpholin-4-yl-7H-pyrrolo[2,3-h]quinazolin-2-yl)aniline (22 mg, 0.06 mmol) in CH₂Cl₂ (1 mL) were added Et₃N (25 μL, 0.18 mmol) and triphosgene (35 mg, 0.12 mmol). A methylamine solution in THF (2 M, 0.09 mL, 0.18 mmol) was added to the mixture after 15 min, and the resulting mixture was stirred at room temperature for 6 h. The solvent was removed under reduced pressure, and the residue was subjected to HPLC separation to give the title compound as off-white solid (TFA salt, 21.4 mg, 67% yield). MS (ESI) m/z 417.4.

### Example 3

### Preparation of 1-ethyl-3-[4-(7-methyl-4-morpholin-4-yl-7H-pyrrolo[2,3-h]quinazolin-2-yl)phenyl]urea

The title compound was prepared by following the procedure as outlined in Example 2. 4-(7-methyl-4-morpholin-4-yl-7H-pyrrolo[2,3-h]quinazolin-2-yl)aniline (22 mg, 0.06 mmol) and ethylamine (2 M in THF, 0.09 mL, 0.18 mmol) gave the title compound as off-white solid (TFA salt, 5.6 mg, 17% yield). MS (ESI) m/z 431.4.

### Example 4

### Preparation of 1-[2-(dimethylamino)ethyl]-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and N,N-dimethylethylenediamine (16 mg, 0.18 mmol) gave the title compound as off-white solid (TFA salt, 21.4 mg, 61% yield). MS (ESI) m/z 474.8.

### Example 5

### Preparation of 1-[3-(dimethylamino)propyl]-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 3-(dimethylamino)-1-propylamine (18 mg, 0.18 mmol) gave the title compound as off-white solid (TFA salt, 29.1 mg, 81% yield). MS (ESI) m/z 488.9.

### Example 6

### Preparation of 4-methyl-N-[4-(7-methyl-4-morpholin-4-yl-7H-pyrrolo[2,3-h]quinazolin-2-yl)phenyl]piperazine-1-carboxamide

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 1-methylpiperazine (18 mg, 0.18 mmol) gave the title compound as off-white solid (TFA salt, 35.6 mg, 99% yield). MS (ESI) m/z 486.5.

### Example 7

### Preparation of 1-(2-furylmethyl)-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and furfurylamine (18 mg, 0.18 mmol) gave the title compound as off-white solid (TFA salt, 24.5 mg, 68% yield). MS (ESI) m/z 483.4.

### Example 8

### Preparation of 1-[3-(1H-imidazol-1-yl)propyl]-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 1-(3-aminopropyl)imidazole (23 mg, 0.18 mmol) gave the title compound as off-white solid (TFA salt, 20.7 mg, 55% yield). MS (ESI) m/z 511.9.

### Example 9

### Preparation of 1-[4-(7-methyl-4-morpholin-4-yl-7H-pyrrolo[2,3-h]quinazolin-2-yl)phenyl]-3-pyridin-2-ylurea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 2-aminopyridine (18 mg, 0.18 mmol) gave the title compound as off-white solid (TFA salt, 12.4 mg, 35% yield). MS (ESI) m/z 480.4.

### Example 10

### Preparation of 1-[4-(7-methyl-4-morpholin-4-yl-7H-pyrrolo[2,3-h]quinazolin-2-yl)phenyl]-3-phenylurea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 2-aminopyridine (18 mg, 0.18 mmol) gave the title compound as off-white solid (TFA salt, 24.3 mg, 67% yield). MS (ESI) m/z 479.5.

HRMS: calcd for C₂₈H₂₆N₆O₂+H⁺, 479.21900; found (ESI-FTMS, [M+H]1+), 479.21743.

### Example 11

### Preparation of 1-[4-(7-methyl-4-morpholin-4-yl-7H-pyrrolo[2,3-h]quinazolin-2-yl)phenyl]-3-pyridin-4-ylurea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 4-aminopyridine (18 mg, 0.18 mmol) gave the title compound as off-white solid (TFA salt, 32.3 mg, 91% yield). MS (ESI) m/z 480.3.

HRMS: calcd for C₂₇H₂₅N₇O₂+H⁺, 480.21425; found (ESI-FTMS, [M+H]1+), 480.21209.

### Example 12

### Preparation of 1-(4-isopropylphenyl)-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 4-isopropylaniline (24 mg, 0.18 mmol) gave the title compound as off-white solid (TFA salt, 5.7 mg, 15% yield). MS (ESI) m/z 521.5.

### Example 13

### Preparation of 1-(3-chlorophenyl)-3-[4-(7-methyl-4-morpholin-4-yl-7H-pyrrolo[2,3-h]quinazolin-2-yl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 3-chloroaniline (23 mg, 0.18 mmol) gave the title compound as off-white solid (TFA salt, 21.9 mg, 58% yield). MS (ESI) m/z 513.4.

### Example 14

### Preparation of 1-[4-(7-methyl-4-morpholin-4-yl-7H-pyrrolo[2,3-h]quinazolin-2-yl)phenyl]-3-[4-(trifluoromethyl)phenyl]urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 4-(trifluoromethyl)aniline (29 mg, 0.18 mmol) gave the title compound as off-white solid (TFA salt, 11.1 mg, 28% yield). MS (ESI) m/z 547.5.

### Example 15

### Preparation of 1-[4-(7-methyl-4-morpholin-4-yl-7H-pyrrolo[2,3-h]quinazolin-2-yl)phenyl]-3-(pyridin-2-ylmethyl)urea

The title compound was prepared by following the procedure of Example 2. 4-(7-methyl-4-morpholin-4-yl-7H-pyrrolo[2,3-h]quinazolin-2-yl)aniline (22 mg, 0.06 mmol) and 2-(aminomethyl)pyridine (19 mg, 0.18 mmol) gave the title compound as off-white solid (TFA salt, 24.2 mg, 66% yield). MS (ESI) m/z 494.5.

### Example 16

### Preparation of N-[4-(7-methyl-4-morpholin-4-yl-7H-pyrrolo[2,3-h]quinazolin-2-yl)phenyl]acetamide

To a solution of 4-(7-methyl-4-morpholin-4-yl-7H-pyrrolo[2,3-h]quinazolin-2-yl)aniline (22 mg, 0.06 mmol) in CH₂Cl₂ (1 mL) were added Et₃N (25 uL, 0.18 mmol) and acetyl chloride (10 mg, 0.12 mmol). The resulting mixture was stirred at room temperature for 6 h. The solvent was removed under reduced pressure, and the residue was subjected to HPLC separation to give the title compound as off-white solid (TFA salt, 22 mg, 71% yield). MS (ESI) m/z 402.5.

### Example 17

### Preparation of N-[4-(7-methyl-4-morpholin-4-yl-7H-pyrrolo[2,3-h]quinazolin-2-yl)phenyl]nicotinamide

The title compound was prepared by following the procedure of Example 16. 4-(7-methyl-4-morpholin-4-yl-7H-pyrrolo[2,3-h]quinazolin-2-yl)aniline (22 mg, 0.06 mmol) and nicotinoyl chloride hydrochloride (21 mg, 0.12 mmol) gave the title compound as off-white solid (TFA salt, 27.5 mg, 79% yield). MS (ESI) m/z 465.6.

### Example 18

### Preparation of N-[4-(7-methyl-4-morpholin-4-yl-7H-pyrrolo[2,3-h]quinazolin-2-yl)phenyl]isonicotinamide

The title compound was prepared by following the procedure of Example 16. 4-(7-methyl-4-morpholin-4-yl-7H-pyrrolo[2,3-h]quinazolin-2-yl)aniline (22 mg, 0.06 mmol) and isonicotinoyl chloride hydrochloride (21 mg, 0.12 mmol) gave the title compound as off-white solid (TFA salt, 25.2 mg, 73% yield). MS (ESI) m/z 465.6.

### Example 19

### Preparation of 4-fluoro-N-[4-(7-methyl-4-morpholin-4-yl-7H-pyrrolo[2,3-h]quinazolin-2-yl)phenyl]benzamide

The title compound was prepared by following the procedure of Example 16. 4-(7-methyl-4-morpholin-4-yl-7H-pyrrolo[2,3-h]quinazolin-2-yl)aniline (22 mg, 0.06 mmol) and 4-fluorobenzoyl chloride (19 mg, 0.12 mmol) gave the title compound as off-white solid (TFA salt, 21.3 mg, 60% yield). MS (ESI) m/z 482.5.

### Example 20

### Preparation of ethyl [4-(7-methyl-4-morpholin-4-yl-7H-pyrrolo[2,3-h]quinazolin-2-yl)phenyl]carbamate

The title compound was prepared by following the procedure of Example 16. 4-(7-methyl-4-morpholin-4-yl-7H-pyrrolo[2,3-h]quinazolin-2-yl)aniline (22 mg, 0.06 mmol) and ethyl chloroformate (11 mg, 0.12 mmol) gave the title compound as off-white solid (TFA salt, 25.8 mg, 79% yield). MS (ESI) m/z 432.4.

### Example 19

### N-[4-(7-methyl-4-morpholin-4-yl-7H-pyrrolo[2,3-h]quinazolin-2-yl)phenyl]methanesulfonamide

To a solution of 4-(7-methyl-4-morpholin-4-yl-7H-pyrrolo[2,3-h]quinazolin-2-yl)aniline (22 mg, 0.06 mmol) in CH₂Cl₂ (1 mL) was added Et₃N (25 uL, 0.18 mmol) and methanesulfonyl chloride (14 mg, 0.12 mmol). The resulting mixture was stirred at room temperature for 6 h. The solvent was removed under reduced pressure, and the residue was treated with 0.5 ml 5M NaOH and MeOH (2 mL). The mixture was stirred at room temperature for 7 h, and concentrated under reduced pressure. The residue was subjected to HPLC separation to give the title compound as off-white solid (TFA salt, 6.7 mg, 26% yield). MS (ESI) m/z 438.5.

### Example 22

### N-[4-(7-methyl-4-morpholin-4-yl-7H-pyrrolo[2,3-h]quinazolin-2-yl)phenyl]benzenesulfonamide

The title compound was prepared by following the procedure of Example 21. 4-(7-methyl-4-morpholin-4-yl-7H-pyrrolo[2,3-h]quinazolin-2-yl)aniline (22 mg, 0.06 mmol) and benzenesulfonyl chloride (21 mg, 0.12 mmol) gave the title compound as off-white solid (TFA salt, 18.2 mg, 61% yield). MS (ESI) m/z 500.5.

### Example 23

### Preparation of 3-(7-methyl-4-morpholin-4-yl-7H-pyrrolo[2,3-h]quinazolin-2-yl)benzaldehyde

A mixture of 2-chloro-7-methyl-4-morpholin-4-yl-7H-pyrrolo[2,3-h]quinazoline (550 mg, 1.8 mmol), 3-formylphenylboronic acid (409 mg, 2.7 mmol), Pd(PPh₃)₄ (105 mg, 5 mol %), dimethoxyethane (DME, 4 mL) and 2M Na₂CO₃ (3 mL) was heated at 120° C. for 0.5 h in microwave oven. The reaction mixture was cooled to room temperature, and filtered through a pad of Celite™, washed with THF. The filtrate was concentrated under reduced pressure, and the residue was subjected to flash chromatography in silica gel (EtOAc:Hexane=50:50) to give the title compound as off-white solid (407 mg, 60% yield). MS (ESI) m/z 373.3.

HRMS: calcd for C₂₂H₂₀N₄O₂+H⁺, 373.16590; found (ESI-FTMS, [M+H]1+), 373.16632.

### Example 24

### Preparation of 1-[3-(7-methyl-4-morpholin-4-yl-7H-pyrrolo[2,3-h]quinazolin-2-yl)phenyl]ethanol

To a solution of 3-(7-methyl-4-morpholin-4-yl-7H-pyrrolo[2,3-h]quinazolin-2-yl)benzaldehyde (22 mg, 0.06 mmol) in THF (2 mL) was added methyl magnesium bromide (2M in THF, 0.09 mL, 0.18 mmol) at −78° C. The resulting mixture was stirred at −78° C. for 3 h, and then quenched by addition of 1 mL of saturated ammonium chloride aqueous solution. The mixture was allowed to warm to room temperature, and extracted with EtOAc. The combined organic layers were washed with water and brine, dried over MgSO₄. The solvent was removed under reduced pressure, and the residue was subjected to HPLC to give the title compound as yellow solid (TFA salt, 10 mg, 33% yield). MS (ESI) m/z 389.3.

### Example 25

### 1-[3-(7-methyl-4-morpholin-4-yl-7H-pyrrolo[2,3-h]quinazolin-2-yl)phenyl]propan-1-ol

The title compound was prepared by following the procedure of Example 24. 3-(7-methyl-4-morpholin-4-yl-7H-pyrrolo[2,3-h]quinazolin-2-yl)benzaldehyde (22 mg, 0.06 mmol) and ethyl magnesium bromide (2M in THF, 0.09 mL, 0.18 mmol) gave the title compound as yellow solid (TFA salt, 18.5 mg, 60% yield). MS (ESI) m/z 403.6.

### Example 26

### 1-[3-(7-methyl-4-morpholin-4-yl-7H-pyrrolo[2,3-h]quinazolin-2-yl)phenyl]prop-2-en-1-ol

The title compound was prepared by following the procedure of Example 24. 3-(7-methyl-4-morpholin-4-yl-7H-pyrrolo[2,3-h]quinazolin-2-yl)benzaldehyde (22 mg, 0.06 mmol) and vinyl magnesium chloride (2M in THF, 0.09 mL, 0.18 mmol) gave the title compound as yellow solid (TFA salt, 16.6 mg, 54% yield). MS (ESI) m/z 401.4.

### Example 27

### 1-[3-(7-methyl-4-morpholin-4-yl-7H-pyrrolo[2,3-h]quinazolin-2-yl)phenyl]but-3-en-1-ol

The title compound was prepared by following the procedure of Example 24. 3-(7-methyl-4-morpholin-4-yl-7H-pyrrolo[2,3-h]quinazolin-2-yl)benzaldehyde (22 mg, 0.06 mmol) and allyl magnesium chloride (2M in THF, 0.09 mL, 0.18 mmol) gave the title compound as yellow solid (TFA salt, 10.6 mg, 33% yield). MS (ESI) m/z 415.4.

### Example 28

### 3-methyl-1-[3-(7-methyl-4-morpholin-4-yl-7H-pyrrolo[2,3-h]quinazolin-2-yl)phenyl]butan-1-ol

The title compound was prepared by following the procedure of Example 24. 3-(7-methyl-4-morpholin-4-yl-7H-pyrrolo[2,3-h]quinazolin-2-yl)benzaldehyde (22 mg, 0.06 mmol) and isobutyl magnesium bromide (2M in THF, 0.09 mL, 0.18 mmol) gave the title compound as yellow solid (TFA salt, 16.8 mg, 51% yield). MS (ESI) m/z 431.4.

### Example 29

### [3-(7-methyl-4-morpholin-4-yl-7H-pyrrolo[2,3-h]quinazolin-2-yl)phenyl](phenyl)methanol

The title compound was prepared by following the procedure of Example 24. 3-(7-methyl-4-morpholin-4-yl-7H-pyrrolo[2,3-h]quinazolin-2-yl)benzaldehyde (22 mg, 0.06 mmol) and phenyl magnesium bromide (2M in THF, 0.09 mL, 0.18 mmol) gave the title compound as yellow solid (TFA salt, 20.4 mg, 60% yield). MS (ESI) m/z 451.4.

### Example 30

### (3-{7-[2-(dimethylamino)ethyl]-4-morpholin-4-yl-7H-pyrrolo[2,3-h]quinazolin-2-yl}phenyl)methanol

To a solution of 2-chloro-4-morpholin-4-yl-7H-pyrrolo[2,3h]quinazoline (500 mg, 1.7 mmol) in DMF (15 mL) were added Cs₂CO₃ (1.425 g, 4.4 mmol) and 2-(dimethylamino)ethyl chloride hydrochloride (490 mg, 3.4 mmol). The mixture was heated at 80° C. overnight. The mixture was cooled to room temperature, and water was added, extracted with EtOAc. The combined extracts were washed with brine, and dried over MgSO₄. The solvent was removed under reduced pressure to give the crude intermediate 2-chloro-7-(2-(dimethylamino)ethyl)-4-morpholin-4-yl-7H-pyrrolo[2,3h]quinazoline (424 mg, 68% yield), which was used in next step without further purification. MS (ESI) m/z 360.4.

A mixture of 2-chloro-7-(2-(dimethylamino)ethyl)-4-morpholin-4-yl-7H-pyrrolo[2,3h]quinazoline (62 mg, 0.17 mmol), 3-(hydroxymethyl)phenylboronic acid (39 mg, 0.26 mmol), Pd(PPh₃)₄ (10 mg, 5 mol %), dimethoxyethane (DME, 3 mL) and 2M Na₂CO₃ (0.5 mL) was heated at 130° C. for 0.5 h in microwave oven. The reaction mixture was cooled to room temperature, and filtered through a pad of Celite™, washed with THF. The filtrate was concentrated under reduced pressure, and the residue was subjected to HPLC separation to give the title compound as yellow solid (TFA salt, 72 mg, 78% yield). MS (ESI) m/z 432.4.

### Example 31

### 2-{2-[3-(benzyloxy)phenyl]-4-morpholin-4-yl-7H-pyrrolo[2,3-h]quinazolin-7-yl}-N,N-dimethylethanamine

The title compound was prepared by following the procedure of Example 30. Suzuki reaction of 2-chloro-7-(2-(dimethylamino)ethyl)-4-morpholin-4-yl-7H-pyrrolo[2,3h]quinazoline (124 mg, 0.34 mmol) and 3-benzyloxyphenylboronic acid (118 mg, 0.52 mmol) gave the title compound as yellow solid (96 mg, 56% yield). MS (ESI) m/z 508.4.

### Example 32

### 3-{7-[2-(dimethylamino)ethyl]-4-morpholin-4-yl-7H-pyrrolo[2,3-h]quinazolin-2-yl}phenol

A mixture of 2-{2-[3-(benzyloxy)phenyl]-4-morpholin-4-yl-7H-pyrrolo[2,3-h]quinazolin-7-yl}-dimethylethanamine (40 mg, 0.08 mmol) and 10% Pd/C (20 mg) in MeOH (10 mL) was stirred at room temperature under hydrogen (50 psi) overnight. The mixture was filtered through a pad of Celite™ and washed with THF and MeOH, and the filtrate was concentrated under reduced pressure. The residue was subjected to HPLC separation to give the title compound as yellow solid (28 mg, 86% yield). MS (ESI) m/z 418.4.

### Example 33

### 1-(4-{7-[2-(dimethylamino)ethyl]-4-morpholin-4-yl-7H-pyrrolo[2,3-h]quinazolin-2-yl}phenyl)-3-pyridin-4-ylurea

The title compound was prepared by following the procedure of Example 30. Suzuki reaction of 2-chloro-7-(2-(dimethylamino)ethyl)-4-morpholin-4-yl-7H-pyrrolo[2,3h]quinazoline (32 mg, 0.09 mmol) and 1-(pyridine-4-yl)-3-(4-(4,4,5,5-tetramethyl-1,3,2-dioxaborolan-2-yl)phenylurea (61 mg, 0.18 mmol) gave the title compound as yellow solid (21 mg, 44% yield). MS (ESI) m/z 537.5.

### Example 34

### 5-{7-[2-(dimethylamino)ethyl]-4-morpholin-4-yl-7H-pyrrolo[2,3-h]quinazolin-2-yl}pyrimidin-2-amine

The title compound was prepared by following the procedure of Example 30. Suzuki reaction of 2-chloro-7-(2-(dimethylamino)ethyl)-4-morpholin-4-yl-7H-pyrrolo[2,3h]quinazoline (32 mg, 0.09 mmol) and 2-aminopyrimidin-5-ylboronic acid (25 mg, 0.18 mmol) gave the title compound as yellow solid (15 mg, 40% yield). MS (ESI) m/z 419.4.

### Example 35

### Preparation of 3-(7-methyl-4-morpholin-4-yl-7H-pyrrolo[2,3-h]quinazolin-2-yl)phenol

A mixture of 7-methyl-2-chloro-4-morpholin-4-yl-7H-pyrrolo[2,3h]quinazoline (200 mg, 0.66 mmol), 3-hydroxyphenylboronic acid (220 mg, 1.7 mmol) Pd(PPh₃)₄ (50 mg) and 2M solution of Na₂CO₃ (3 ml) was heated at reflux in dimethoxyethane (30 ml) for 24 h under nitrogen atmosphere. At the end, reaction mixture was filtered through Celite™, washed well with chloroform and extracted with chloroform. Organic layer was washed with water; dried over anhydrous MgSO₄ and concentrated. The product was purified by SiO₂ column chromatography by eluting it with ethyl acetate: methanol (98:2). Yield: 130 mg, 56%; (M+H) 361.416.

### Example 36

### Preparation of Methyl 4-(7-methyl-4-morpholin-4-yl-7H-pyrrolo[2,3-h]quinazolin-2-yl)benzoate

Starting from a mixture of 7-methyl-2-chloro-4-morpholin-4-yl-7H pyrrolo[2,3h]quinazoline (440 mg, 1.45 mmol), 4-carbethoxymethylphenylboronic acid (650 mg, 3.6 mmol) Pd(PPh₃)₄ (50 mg) and 2M solution of Na₂CO₃ (3 ml) and following the procedure as outlined in Example 35, the titled compound was isolated white solid. Yield: 450 mg, 77%; (M+H) 403.

### Example 37

### Preparation of 3-(7-methyl-4-morpholin-4-yl-7H-pyrrolo[2,3-h]quinazolin-2-yl)phenyl]methanol

Starting from a mixture of 7-methyl-2-chloro-4-morpholin-4-yl-7H pyrrolo[2,3h]quinazoline (440 mg, 1.45 mmol), 3-hydroxymethyl phenylboronic acid (550 mg, 3.6 mmol) Pd(PPh₃)₄ (50 mg) and 2M solution of Na₂CO₃ (3 ml) and following the procedure as outlined in Example 35, the titled compound was isolated white solid. Yield: 480 mg, 88%; (M+H) 362.3.

### Example 38

### Preparation of 4-(7-methyl-4-morpholin-4-yl-7H-pyrrolo[2,3-h]quinazolin-2-yl)benzoic acid

To a stirred solution of MeOH/THF (1:1, 75 ml) methyl 4-(7-methyl-4-morpholin-4-yl-7H-pyrrolo[2,3-h]quinazolin-2-yl)benzoate (110 mg, 0.27 mmol), NaOH (21 mg, 0.52 mmol) was added in 2 ml water. The reaction mixture was stirred for 12 h at room temperature and at the end, reaction mixture was concentrated and was neutralized with con. HCl. The separated white solid was filtered and washed with water. The product was crystallized from aqueous MeOH. Yield: 70 mg, 66%; (M−H) 388.2.

### Example 39

### Preparation of 3-(7-methyl-4-morpholin-4-yl-7H-pyrrolo[2,3-h]quinazolin-2-yl)benzamide

Starting from a mixture of 7-methyl-2-chloro-4-morpholin-4-yl-7H pyrrolo[2,3h]quinazoline (400 mg, 1.32 mmol), 3-carbamoylphenylboronic acid (540 mg, 3.3 mmol) Pd(PPh₃)₄ (50 mg) and 2M solution of Na₂CO₃ (3 ml) and following the procedure as outlined in Example 35, the titled compound was isolated white solid. Yield: 52 mg, 10%; (M+H) 389.3.

### Example 40

### Preparation of 3-(7-methyl-4-morpholin-4-yl-7H-pyrrolo[2,3-h]quinazolin-2-yl)benzonitrile

Starting from a mixture of 7-methyl-2-chloro-4-morpholin-4-yl-7H pyrrolo[2,3h]quinazoline (400 mg, 1.32 mmol), 3-cyanophenylboronic acid (470 mg, 3.3 mmol) Pd(PPh₃)₄ (50 mg) and 2M solution of Na₂CO₃ (3 ml) and following the procedure as outlined in Example 35, the titled compound was isolated white solid. Yield: 150 mg, 27%; (M+H) 371.3.

### Example 41

### Preparation of 3-(7-methyl-4-morpholin-4-yl-7H-pyrrolo[2,3-h]quinazolin-2-yl)aniline

Starting from a mixture of 7-methyl-2-chloro-4-morpholin-4-yl-7H pyrrolo[2,3h]quinazoline (300 mg, 1 mmol), 3-aminophenylboronic acid (290 mg, 2.5 mmol) Pd(PPh₃)₄ (50 mg) and 2M solution of Na₂CO₃ (3 ml) and following the procedure as outlined in Example 35, the titled compound was isolated white solid. Yield: 48 mg, 12%; (M+H) 348.3.

### Example 42

### Preparation of 5-(7-methyl-4-morpholin-4-yl-7H-pyrrolo[2,3-h]quinazolin-2-yl)pyridin-2-amine

Starting from a mixture of 7-methyl-2-chloro-4-morpholin-4-yl-7H pyrrolo[2,3h]quinazoline (150 mg, 0.5 mmol), 6-aminopyridyl-3-boronic acid (270 mg, 1.23 mmol) Pd(PPh₃)₄ (50 mg) and 2M solution of Na₂CO₃ (3 ml) and following the procedure as outlined in Example 35, the titled compound was isolated white solid. Yield: 48 mg, 12%; (M+H) 361.4.

### Example 43

### Preparation of 5-(7-methyl-4-morpholin-4-yl-7H-pyrrolo[2,3-h]quinazolin-2-yl)pyrimidin-2-amine

Starting from a mixture of 7-methyl-2-chloro-4-morpholin-4-yl-7H pyrrolo[2,3h]quinazoline (150 mg, 0.5 mmol), 2-aminopyrimidyl-5-boronic acid (270 mg, 1.94 mmol) Pd(PPh₃)₄ (50 mg) and 2M solution of Na₂CO₃ (3 ml) and following the procedure as outlined in Example 35, the titled compound was isolated white solid. Yield: 15 mg, 9%; (M+H) 362.3.

### Example 44

### Preparation of N-[3-(7-methyl-4-morpholin-4-yl-7H-pyrrolo[2,3-h]quinazolin-2-yl)benzyl]acetamide

To a stirred solution of N-[3-(7-methyl-4-morpholin-4-yl-7H-pyrrolo[2,3-h]quinazolin-2-yl)benzyl amine (150 mg. 0.4 mmol) in methylene chloride, acetyl chloride (40 mg, 0.52 mmol) was added at 0° C. and slowly brought to room temperature. It was stirred for 2 h and quenched with water. Reaction mixture was washed with saturated NaHCO₃ and dried over anhydrous MgSO₄. It was filtered and concentrated. The product was purified by silica-gel column chromatography (2:1 Hexane/ethyl acetate) to give a white solid. Yield: 30 mg, 15%; (M+H) 416.5.

### Example 45

### Preparation of 2-(1H-indol-4-yl)-7-methyl-4-morpholin-4-yl-7H-pyrrolo[2,3-h]quinazoline

Starting from a mixture of 7-methyl-2-chloro-4-morpholin-4-yl-7H pyrrolo[2,3h]quinazoline (150 mg, 0.5 mmol), 1H-indolyl-4-boronic acid (200 mg, 1.24 mmol) Pd(PPh₃)₄ (50 mg) and 2M solution of Na₂CO₃ (3 ml) and following the procedure as outlined in Example 35, the titled compound was isolated white solid. Yield: 20 mg, 10%; (M+H) 384.3.

### Example 46

### Preparation of 3-(7-benzyl-4-morpholin-4-yl-7H-pyrrolo[2,3-h]quinazolin-2-yl)phenol

Step 1: A mixture of 4-amino-1-benzylindole (4.6 g, 20 mmol) and trichloromethyl isocyanate (3.5 g, 22 mmol) was stirred in anhydrous dioxane for 48 h at room temperature. At the end, reaction mixture was concentrated and the separated sticky mass was dissolved in chloroform and triturated with diethyl ether and stirred at room temperature. The separated product, 7-benzyl-4-chloro-1,7-dihydro-2H-pyrrolo[2,3-h]quinazoline-2-one was filtered and washed with ether. Yield: 3.0 g, 49%; (M+H) 310.7.

Step 2: A mixture of 7-benzyl-4-chloro-1,7-dihydro-2H-pyrrolo[2,3-h]quinazoline-2-one (1.0 g, 3.23 mmol) and POCl₃ (80 ml) was heated at 80° C. for 1 h. The reaction mixture was then concentrated to dryness and quenched with ice-cold water. The product was carefully neutralized with NH₄OH and the separated solid, 7-benzyl-2,4-dichloro-7H-pyrrolo[2,3-h]quinazoline, was filtered, dried and used for further transformation without purification. Yield: 980 mg, 92%; (M+H) 329.2.

Step 3: A mixture of 7-benzyl-2,4-dichloro-7H-pyrrolo[2,3-h]quinazoline (1.0 g, 3.1 mmol), morpholine (261 mg, 3.2 mmol) and triethylamine (1 ml) was stirred in chloroform solution at room temperature for 6 h. At the end, reaction mixture was quenched with water; washed well with water and dried over anhydrous MgSO₄. It was filtered and concentrated. The crude product was purified by SiO₂ column chromatography by eluting it with 1:1 EtOAc:Hexane. Yield: 500 mg, 44%; (M+H) 379.8.

Step 4: Starting from a mixture of 7-benzyl-2-chloro-4-morpholin-4-yl-7H pyrrolo[2,3-h]quinazoline (100 mg, 0.26 mmol), 3-hydroxy phenylboronic acid (100 mg, excess) Pd(PPh₃)₄ (50 mg) and 2M solution of Na₂CO₃ (3 ml) and following the procedure as outlined in Example 35, the titled compound was isolated white solid. Yield: 66 mg, 58%; (M+H) 437.5.

### Example 47

### Preparation of 2-(6-methoxypyridin-3-yl)-7-methyl-4-morpholin-4-yl-7H-pyrrolo[2,3-h]quinazoline

Starting from a mixture of 7-methyl-2-chloro-4-morpholin-4-yl-7H pyrrolo[2,3h]quinazoline (400 mg, 1.32 mmol), pyridyl-2-methoxy-5-boronic acid (400 mg, 2.9 mmol) Pd(PPh₃)₄ (50 mg) and 2M solution of Na₂CO₃ (3 ml) and following the procedure as outlined in Example 35, the titled compound was isolated white solid. Yield: 180 mg, 32%; (M+H) 376.1.

### Example 48

### Preparation of 5-(7-methyl-4-morpholin-4-yl-7H-pyrrolo[2,3-h]quinazolin-2-yl)pyridin-2-ol

2-(6-methoxypyridin-3-yl)-7-methyl-4-morpholin-4-yl-7H-pyrrolo[2,3-h]quinazoline (100 mg. 0.26 mmol) was dissolved in conc. HCl (5 ml) and methanol (10 ml) and heated at reflux for 4 h. At the end reaction mixture was concentrated and neutralized with NH₄OH. Separated solid was dissolved in chloroform; washed well with water; dried over anhydrous MgSO₄ and concentrated. The product was purified by SiO₂ column chromatography by eluting it with EtOAc:MeOH (95:5). Yield: 81 mg, 84%; (M+H) 362.3.

### Example 49

### Preparation of 2-(1H-indol-4-yl)-7-methyl-4-morpholin-5-yl-7H-pyrrolo[2,3-h]quinazoline

Starting from a mixture of 7-methyl-2-chloro-4-morpholin-5-yl-7H pyrrolo[2,3h]quinazoline (483 mg, 1.6 mmol), 1H-indolyl-5-boronic acid (400 mg, 2.5 mmol) Pd(PPh₃)₄ (50 mg) and 2M solution of Na₂CO₃ (3 ml) and following the procedure as outlined in Example 35, the titled compound was isolated white solid. Yield: 20 mg, 10%; (M+H) 384.3.

### Example 50

### Preparation of 2-(2-methoxypyrimidin-5-yl)-7-methyl-4-morpholin-4-yl-7H-pyrrolo[2,3-h]quinazoline

Starting from a mixture of 7-methyl-2-chloro-4-morpholin-5-yl-7H pyrrolo[2,3h]quinazoline (500 mg, 1.7 mmol), 2-methoxypyrimidyl-5-boronic acid (560 mg, 4.1 mmol) Pd(PPh₃)₄ (50 mg) and 2M solution of Na₂CO₃ (3 ml) and following the procedure as outlined in Example 35, the titled compound was isolated white solid. Yield: 250 mg, 32%; (M+H) 377.4.

### Example 51

### Preparation of 5-(7-methyl-4-morpholin-4-yl-7H-pyrrolo[2,3-h]quinazolin-2-yl)pyrimidin-2-ol

2-(2-methoxypyrimidin-5-yl)-7-methyl-4-morpholin-4-yl-7H-pyrrolo[2,3-h]quinazoline (100 mg. 0.26 mmol) was dissolved in conc. HCl (5 ml) and methanol (10 ml) and heated at reflux for 4 h. The reaction mixture was concentrated and neutralized with NH₄OH. The separated solid was filtered and washed well with water. The product was dried, suspended in diethyl ether, and filtered. It was dried and found to be pure. Yield: 80 mg, 83%; (M+H) 363.3.

### Example 52

### Preparation of 2-(3-fluorophenyl)-7-methyl-4-morpholin-4-yl-7H-pyrrolo[2,3-h]quinazoline

Starting from a mixture of 7-methyl-2-chloro-4-morpholin-4-yl-7H pyrrolo[2,3h]quinazoline (150 mg, 0.5 mmol), 3-flourophenylboronic acid (200 mg, 1.43 mmol) Pd(PPh₃)₄ (50 mg) and 2M solution of Na₂CO₃ (3 ml) and following the procedure as outlined in Example 35, the titled compound was isolated white solid. Yield: 38 mg, 21%; (M+H) 363.4.

### Example 53

### Preparation of 4-chloro-3-(7-methyl-4-morpholin-4-yl-7H-pyrrolo[2,3-h]quinazolin-2-yl)phenol

Starting from a mixture of 7-methyl-2-chloro-4-morpholin-4-yl-7H pyrrolo[2,3h]quinazoline (150 mg, 0.5 mmol), 2-chloro-5-hydroxy-phenylboronic acid (200 mg, 1.16 mmol) Pd(PPh₃)₄ (50 mg) and 2M solution of Na₂CO₃ (3 ml) and following the procedure as outlined in Example 35, the titled compound was isolated white solid. Yield: 31 mg, 16%; (M+H) 395.2.

### Example 54

### Preparation of 1-[4-(7-methyl-4-morpholin-4-yl-7H-pyrrolo[2,3-h]quinazolin-2-yl)phenyl]-3-propylurea

Starting from a mixture of 7-methyl-2-chloro-4-morpholin-4-yl-7H pyrrolo[2,3h]quinazoline (500 mg, 1.65 mmol), 4-(3-propylureido)phenylboronic acid, pinacol ester (800 mg, 2.6 mmol) Pd(PPh₃)₄ (50 mg) and 2M solution of Na₂CO₃ (3 ml) and following the procedure as outlined in Example 35, the titled compound was isolated white solid. Yield: 58 mg, 8%; (M+H) 455.4.

### Example 55

### Preparation of N,N-dimethyl-N′-[3-(7-methyl-4-morpholin-4-yl-7H-pyrrolo[2,3-h]quinazolin-2-yl)phenyl]sulfamide

Starting from a mixture of 7-methyl-2-chloro-4-morpholin-4-yl-7H pyrrolo[2,3h]quinazoline (400 mg, 1.32 mmol), 3-(N,N-dimethylsulfamoylamino)phenylboronic acid (800 mg, 2.6 mmol) Pd(PPh₃)₄ (50 mg) and 2M solution of Na₂CO₃ (3 ml) and following the procedure as outlined in Example 35, the titled compound was isolated white solid. Yield: 60 mg, 10%; (M+H) 467.3.

### Example 56

### Preparation of N-cyclopropyl-3-(7-methyl-4-morpholin-4-yl-7H-pyrrolo[2,3-h]quinazolin-2-yl)benzenesulfonamide

Starting from a mixture of 7-methyl-2-chloro-4-morpholin-4-yl-7H pyrrolo[2,3h]quinazoline (530 mg, 1.75 mmol), N-cyclopropyl 3-boronobenzenesulfanamide (800 mg, 3.3 mmol) Pd(PPh₃)₄ (50 mg) and 2M solution of Na₂CO₃ (3 ml) and following the procedure as outlined in Example 35, the titled compound was isolated white solid. Yield: 58 mg, 7%; (M+H) 464.2.

### Example 57

### Preparation of 3-(4-morpholin-4-yl-7H-pyrrolo[2,3-h]quinazolin-2-yl)phenyl]methanol

Starting from a mixture of 2-chloro-4-morpholin-4-yl-7H pyrrolo[2,3h]quinazoline (230 mg, 0.79 mmol), 3-hydroxymethyl phenylboronic acid (300 mg, 1.98 mmol) Pd(PPh₃)₄ (50 mg) and 2M solution of Na₂CO₃ (3 ml) and following the procedure as outlined in Example 35, the titled compound was isolated white solid. Yield: 45 mg, 16%; (M+H) 361.3.

### Example 58

### Preparation of -(4-morpholin-4-yl-7H-pyrrolo[2,3-h]quinazolin-2-yl)pyrimidin-2-amine

Starting from a mixture of 2-chloro-4-morpholin-4-yl-7H pyrrolo[2,3h]quinazoline (150 mg, 0.52 mmol), 2-amino-pyrimidine-5-boronic acid (180 mg, 1.3 mmol) Pd(PPh₃)₄ (50 mg) and 2M solution of Na₂CO₃ (3 ml) and following the procedure as outlined in Example 35, the titled compound was isolated white solid. Yield: 70 mg, 41%; (M+H) 348.3.

### Example 59

### Preparation of 3-[7-(methylsulfonyl)-4-morpholin-4-yl-7H-pyrrolo[2,3-h]quinazolin-2-yl]phenol

To a stirred solution of 2-chloro-4-morpholin-4-yl-7H pyrrolo[2,3h]quinazoline (1.0 g 3.5 mmol) in THF (50 ml) at −78° C., n-butyl lithium (2 ml, 1.6 M solution, 3.2 mmol) was added and kept at this temperature for 20 minutes. Then methane sulfonyl chloride (399 mg, 3.5 mmol) was added in THF solution (1 ml) and stirred at room temperature for 2 h. At the end, reaction mixture was quenched with saturated NH₄Cl and extracted with CH₂Cl₂. It was washed well with water; dried over anhydrous MgSO₄; filtered and concentrated. The crude product obtained was taken to the next step with out purification.

Starting from the crude product obtained above (240 mg, 0.65 mmol), 3-hydroxy-phenyl boronic acid (136 mg, 1.0 mmol) Pd(PPh₃)₄ (50 mg) and 2M solution of Na₂CO₃ (3 ml) and following the procedure as outlined in Example 35, the titled compound was isolated white solid. Yield: 56 mg, 20%; (M+H) 425.2.

### Example 60

### Preparation of tert-butyl [2-(6-aminopyridin-3-yl)-4-morpholin-4-yl-7H-pyrrolo[2,3-h]quinazolin-7-yl]acetate

A mixture of 2-chloro-4-morpholin-4-yl-7H pyrrolo[2,3h]quinazoline (1.0 g, 3.5 mmol), 1-bromo-tert.butyl acetate (740 mg, 3.8 mmol) and anhydrous K₂CO₃ was heated at reflux in acetone for 16 h. At the end, reaction mixture was filtered and concentrated. The residue was extracted with chloroform, washed well with water; dried over anhydrous MgSO₄; filtered and concentrated. The crude product, tert-butyl (2-chloro-4-morpholin-4-yl-7H-pyrrolo[2,3-h]quinazolin-7-yl)acetate (1.15 g, 87%) was taken to next step with out purification.

Starting from the crude product obtained above (500 mg, 1.2 mmol), 2-amino-5-pyridylboronic acid (250 mg, 2.5 mmol) Pd(PPh₃)₄ (50 mg) and 2M solution of Na₂CO₃ (3 ml) and following the procedure as outlined in Example 35, the titled compound was isolated white solid. Yield: 60 mg, 10%; (M+H) 461.4.

### Example 61

### benzyl 4-[(4-morpholin-4-yl-2-{4-[(pyridin-4-ylcarbamoyl)amino]phenyl}-7H-pyrrolo[2,3-h]quinazolin-7-yl)sulfonyl]piperidine-1-carboxylate

MS (ESI) m/z 747.2;

MS (ESI) m/z 374.1;

MS (ESI) m/z 394.6;

HRMS: calcd for C₃₉H₃₈N₈O₆S+H⁺, 747.27078; found (ESI-FTMS, [M+H]1+), 747.27211.

### Example 62

### 1-{4-[4-morpholin-4-yl-7-(piperidin-4-ylsulfonyl)-7H-pyrrolo[2,3-h]quinazolin-2-yl]phenyl}-3-pyridin-4-ylurea

MS (ESI) m/z 613.4;

MS (ESI) m/z 327.7;

MS (ESI) m/z 307.2;

HRMS: calcd for C₃₁H₃₂N₈O₄S+H⁺, 613.23400; found (ESI, [M+H]+Obsd), 613.2333.

### Example 63

### 1-(4-{7-[(1-methylpiperidin-4-yl)sulfonyl]-4-morpholin-4-yl-7H-pyrrolo[2,3-h]quinazolin-2-yl}phenyl)-3-pyridin-4-ylurea

HRMS: calcd for C₃₂H₃₄N₈O₄S+H⁺, 627.24965; found (ESI, [M+H]+Obsd), 627.2491.

### Example 64

### 1-(4-{7-[(1-ethylpiperidin-4-yl)sulfonyl]-4-morpholin-4-yl-7H-pyrrolo[2,3-h]quinazolin-2-yl}phenyl)-3-pyridin-4-ylurea

MS (ESI) m/z 641.4;

MS (ESI) m/z 241.8;

MS (ESI) m/z 321.2;

HRMS: calcd for C₃₃H₃₆N₈O₄S+H⁺, 641.26530; found (ESI, [M+H]+Obsd), 641.2645.

### Example 65

### 1-(4-{7-[(1-isopropylpiperidin-4-yl)sulfonyl]-4-morpholin-4-yl-7H-pyrrolo[2,3-h]quinazolin-2-yl}phenyl)-3-pyridin-4-ylurea

MS (ESI) m/z 655.5;

MS (ESI) m/z 246.5;

MS (ESI) m/z 328.3;

HRMS: calcd for C₃₄H₃₈N₈O₄S+H⁺, 655.28095; found (ESI, [M+H]+Obsd), 655.2802.

### Example 66

### benzyl 4-{[2-(3-hydroxyphenyl)-4-morpholin-4-yl-7H-pyrrolo[2,3-h]quinazolin-7-yl]sulfonyl}piperidine-1-carboxylate

MS (ESI) m/z 628.4;

HRMS: calcd for C₃₃H₃₃N₅O₆S+H⁺, 628.22243; found (ESI, [M+H]+Obsd), 628.2220.

### Example 67

### 3-[4-morpholin-4-yl-7-(piperidin-4-ylsulfonyl)-7H-pyrrolo[2,3-h]quinazolin-2-yl]phenol

MS (ESI) m/z 494.3;

MS (ESI) m/z 268.2;

MS (ESI) m/z 288.7;

HRMS: calcd for C₂₅H₂₇N₅O₄S+H⁺, 494.18565; found (ESI-FTMS, [M+H]1+), 494.18682.

### Example 68

### 2-[3-(benzyloxy)phenyl]-4-morpholin-4-yl-7-(phenylsulfonyl)-7H-pyrrolo[2,3-h]quinazoline

MS (ESI) m/z 577.3.

### Example 69

### 3-[4-morpholin-4-yl-7-(phenylsulfonyl)-7H-pyrrolo[2,3-h]quinazolin-2-yl]phenol

MS (ESI) m/z 487.5

HRMS: calcd for C₂₆H₂₂N₄O₄S+H⁺, 487.14345; found (ESI-FTMS, [M+H]1+), 487.14379.

### Example 70

### {3-[4-morpholin-4-yl-7-(phenylsulfonyl)-7H-pyrrolo[2,3-h]quinazolin-2-yl]phenyl}methanol

MS (ESI) m/z 501.5

HRMS: calcd for C₂₇H₂₄N₄O₄S+H⁺, 501.15910; found (ESI-FTMS, [M+H]1+), 501.16003.

### Example 71

### 5-[4-morpholin-4-yl-7-(phenylsulfonyl)-7H-pyrrolo[2,3-h]quinazolin-2-yl]pyrimidin-2-amine

MS (ESI) m/z 488.4

HRMS: calcd for C₂₄H₂₁N₇O₃S+H⁺, 488.14993; found (ESI-FTMS, [M+H]1+), 488.151.

### Example 72

### 2-(1H-indazol-4-yl)-4-morpholin-4-yl-7-(phenylsulfonyl)-7H-pyrrolo[2,3-h]quinazoline

MS (ESI) m/z 511.3.

### Example 73

### 5-[7-(methylsulfonyl)-4-morpholin-4-yl-7H-pyrrolo[2,3-h]quinazolin-2-yl]pyrimidin-2-amine

MS (ESI) m/z 426.2

HRMS: calcd for C₁₉H₁₉N₇O₃S+H⁺, 426.13428; found (ESI-FTMS, [M+H]1+), 426.13519.

### Example 74

### {3-[7-(methylsulfonyl)-4-morpholin-4-yl-7H-pyrrolo[2,3-h]quinazolin-2-yl]phenyl}methanol

MS (ESI) m/z 439.3

HRMS: calcd for C₂₂H₂₂N₄O₄S+H⁺, 439.14345; found (ESI, [M+H]+Obsd), 439.1434.

### Example 75

### 2-[5-(methoxymethoxy)pyridin-3-yl]-7-(methylsulfonyl)-4-morpholin-4-yl-7H-pyrrolo[2,3-h]quinazoline

MS (ESI) m/z 470.3

HRMS: calcd for C₂₂H₂₃N₅O₅S+H⁺, 470.14927; found (ESI, [M+H]+Obsd), 470.1499.

### Example 76

### 5-[7-(methylsulfonyl)-4-morpholin-4-yl-7H-pyrrolo[2,3-h]quinazolin-2-yl]pyridin-3-ol

MS (ESI) m/z 426.3

HRMS: calcd for C₂₀H₁₉N₅O₄S+H⁺, 426.12305; found (ESI, [M+H]+Obsd), 426.1229.

### Example 77

### 2-[5-(methoxymethoxy)pyridin-3-yl]-7-methyl-4-morpholin-4-yl-7H-pyrrolo[2,3-h]quinazoline

MS (ESI) m/z 406.2;

HRMS: calcd for C₂₂H₂₃N₅O₃+H⁺, 406.18737; found (ESI-FTMS, [M+H]1+), 406.18738.

### Example 78

### 5-(7-methyl-4-morpholin-4-yl-7H-pyrrolo[2,3-h]quinazolin-2-yl)pyridin-3-ol

MS (ESI) m/z 362.2;

HRMS: calcd for C₂₀H₁₉N₅O₂+H⁺, 362.16115; found (ESI-FTMS, [M+H]1+), 362.16209.

### Example 79

### 2-(1H-indazol-4-yl)-7-methyl-4-morpholin-4-yl-7H-pyrrolo[2,3-h]quinazoline

MS (ESI) m/z 385.3.

### Example 80

### 2-(1H-indazol-4-yl)-4-morpholin-4-yl-7H-pyrrolo[2,3-h]quinazoline

MS (ESI) m/z 371.3.

### Example 81

### 2-[3-(benzyloxy)phenyl]-4-morpholin-4-yl-7H-pyrrolo[2,3-h]quinazoline

MS (ESI) m/z 437.6.

### Example 82

### 2-(3-hydroxyphenyl)-4-morpholin-4-yl-7H-pyrrolo[2,3-h]quinazoline-9-carbaldehyde

MS (ESI) m/z 375.3;

HRMS: calcd for C₂₁H₁₈N₄O₃+H⁺, 375.14517; found (ESI, [M+H]+Obsd), 375.1455.

### Example 83

### Preparation of [3-(7-benzyl-4-morpholin-4-yl-7H-pyrrolo[2,3-h]quinazolin-2-yl)phenyl]methanol

Starting from a mixture of 7-benzyl-2-chloro-4-morpholin-4-yl-7H pyrrolo[2,3h]quinazoline (70 mg, 0.185 mmol), 3-hydroxymethyl phenylboronic acid (100 mg, excess) Pd(PPh₃)₄ (50 mg) and 2M solution of Na₂CO₃ (3 ml) and following the procedure as outlined in Example 35, the titled compound was isolated white solid. Yield: 50 mg, 60%; (M+H) 451.5.

### Example 84

### 1-{4-[7-(methylsulfonyl)-4-morpholin-4-yl-7H-pyrrolo[2,3-h]quinazolin-2-yl]phenyl}-3-phenylurea

4-(2-Chloro-7-(methylsulfonyl)-7H-pyrrolo[2,3-h]quinazolin-4-yl)morpholine was prepared by following the procedure described for example 59.

4-(7-(methylsulfonyl)-4-morpholino-7H-pyrrolo[2,3-h]quinazolin-2-yl)aniline was prepared by following the procedure described for example 30. A mixture of 4-(2-chloro-7-(methylsulfonyl)-7H-pyrrolo[2,3-h]quinazolin-4-yl)morpholine (0.17 mmol), 4-aminophenylboronic acid, pinacol ester (0.26 mmol), Pd(PPh₃)₄ (10 mg, 5 mol %), dimethoxyethane (DME, 3 mL) and 2M Na₂CO₃ (0.5 mL) was heated at 130° C. for 0.5 hours in microwave oven. The reaction mixture was cooled to room temperature, and filtered through a pad of Celite™, washed with THF. The filtrate was concentrated under reduced pressure, and the residue was subjected to HPLC separation.

To a solution of 4-(7-(methylsulfonyl)-4-morpholino-7H-pyrrolo[2,3-h]quinazolin-2-yl)aniline (0.06 mmol) in CH₂Cl₂ (1 mL) were added Et₃N (25 μL, 0.18 mmol) and phenylisocyanate (0.1 mmol), and the resulting mixture was stirred at room temperature for 6 hours. The solvent was removed under reduced pressure, and the residue was subjected to HPLC separation to give the title compound. MS (ESI) m/z

### Example 85

### 1-{4-[7-(methylsulfonyl)-4-morpholin-4-yl-7H-pyrrolo[2,3-h]quinazolin-2-yl]phenyl}-3-pyridin-3-ylurea

The title compound was prepared by following the procedure as outlined in Example 2 using 4-(7-(methylsulfonyl)-4-morpholino-7H-pyrrolo[2,3-h]quinazolin-2-yl)aniline, triphosgene and 3-aminopyridine. MS (ESI) m/z

### Example 86

### ethyl {4-[7-(methylsulfonyl)-4-morpholin-4-yl-7H-pyrrolo[2,3-h]quinazolin-2-yl]phenyl}carbamate

The title compound was prepared by following the procedure as outlined in Example 16 using 4-(7-(methylsulfonyl)-4-morpholino-7H-pyrrolo[2,3-h]quinazolin-2-yl)aniline and ethyl chloroformate. MS (ESI) m/z

### Example 87

### N-{4-[7-(methylsulfonyl)-4-morpholin-4-yl-7H-pyrrolo[2,3-h]quinazolin-2-yl]phenyl}cyclopropanecarboxamide

The title compound was prepared by following the procedure as outlined in Example 16 using 4-(7-(methylsulfonyl)-4-morpholino-7H-pyrrolo[2,3-h]quinazolin-2-yl)aniline and cyclopropane carbonyl chloride. MS (ESI) m/z

### Example 88

### N-{4-[7-(methylsulfonyl)-4-morpholin-4-yl-7H-pyrrolo[2,3-h]quinazolin-2-yl]phenyl}butanamide

The title compound was prepared by following the procedure as outlined in Example 16 using 4-(7-(methylsulfonyl)-4-morpholino-7H-pyrrolo[2,3-h]quinazolin-2-yl)aniline and butyryl chloride. MS (ESI) m/z

### Example 89

### 1-ethyl-3-{4-[7-(methylsulfonyl)-4-morpholin-4-yl-7H-pyrrolo[2,3-h]quinazolin-2-yl]phenyl}urea

The title compound was prepared by following the procedure as outlined in Example 2 using 4-(7-(methylsulfonyl)-4-morpholino-7H-pyrrolo[2,3-h]quinazolin-2-yl)aniline, triphosgene and ethylamine. MS (ESI) m/z

### Example 90

### methyl {4-[7-(methylsulfonyl)-4-morpholin-4-yl-7H-pyrrolo[2,3-h]quinazolin-2-yl]phenyl}carbamate

The title compound was prepared by following the procedure as outlined in Example 16 using 4-(7-(methylsulfonyl)-4-morpholino-7H-pyrrolo[2,3-h]quinazolin-2-yl)aniline and methyl chloroformate. MS (ESI) m/z

### Example 91

### N-{4-[7-(methylsulfonyl)-4-morpholin-4-yl-7H-pyrrolo[2,3-h]quinazolin-2-yl]phenyl}propanamide

The title compound was prepared by following the procedure as outlined in Example 16 using 4-(7-(methylsulfonyl)-4-morpholino-7H-pyrrolo[2,3-h]quinazolin-2-yl)aniline and propionyl chloride. MS (ESI) m/z

### Example 92

### N-{4-[7-(methylsulfonyl)-4-morpholin-4-yl-7H-pyrrolo[2,3-h]quinazolin-2-yl]phenyl}acetamide

The title compound was prepared by following the procedure as outlined in Example 16 using 4-(7-(methylsulfonyl)-4-morpholino-7H-pyrrolo[2,3-h]quinazolin-2-yl)aniline and acetyl chloride. MS (ESI) m/z

### Example 93

### ethyl (4-{7-[2-(dimethylamino)ethyl]-4-morpholin-4-yl-7H-pyrrolo[2,3-h]quinazolin-2-yl}phenyl)carbamate

4-(7-(2-(dimethylamino)ethyl)-4-morpholino-7H-pyrrolo[2,3-h]quinazolin-2-yl)aniline was prepared by following the procedure described for example 30. A mixture of 2-chloro-7-(2-(dimethylamino)ethyl)-4-morpholin-4-yl-7H-pyrrolo[2,3h]quinazoline (62 mg, 0.17 mmol), 4-aminophenylboronic acid, pinacol ester (0.26 mmol), Pd(PPh₃)₄ (10 mg, 5 mol %), dimethoxyethane (DME, 3 mL) and 2M Na₂CO₃ (0.5 mL) was heated at 130° C. for 0.5 hours in microwave oven. The reaction mixture was cooled to room temperature, and filtered through a pad of Celite™, washed with THF. The filtrate was concentrated under reduced pressure, and the residue was subjected to HPLC separation.

To a solution of 4-(7-(2-(dimethylamino)ethyl)-4-morpholino-7H-pyrrolo[2,3-h]quinazolin-2-yl)aniline (0.06 mmol) in CH₂Cl₂ (1 mL) were added Et₃N (25 μL, 0.18 mmol) and ethyl chloroformate (0.1 mmol), and the resulting mixture was stirred at room temperature for 6 hours. The solvent was removed under reduced pressure, and the residue was subjected to HPLC separation to give the title compound. MS (ESI) m/z

### Example 94

### 1-(4-{7-[2-(dimethylamino)ethyl]-4-morpholin-4-yl-7H-pyrrolo[2,3-h]quinazolin-2-yl}phenyl)-3-ethylurea

The title compound was prepared by following the procedure described above for example 84, using 4-(7-(2-(dimethylamino)ethyl)-4-morpholino-7H-pyrrolo[2,3-h]quinazolin-2-yl)aniline and ethyl isocyanate. MS (ESI) m/z

### Example 95

### 1-(4-{7-[2-(dimethylamino)ethyl]-4-morpholin-4-yl-7H-pyrrolo[2,3-h]quinazolin-2-yl}phenyl)-3-phenylurea

The title compound was prepared by following the procedure described above for example 84, using 4-(7-(2-(dimethylamino)ethyl)-4-morpholino-7H-pyrrolo[2,3-h]quinazolin-2-yl)aniline and phenyl isocyanate. MS (ESI) m/z

### Example 96

### N-(4-{7-[2-(dimethylamino)ethyl]-4-morpholin-4-yl-7H-pyrrolo[2,3-h]quinazolin-2-yl}phenyl)cyclopropanecarboxamide

The title compound was prepared by following the procedure described above for example 16, using 4-(7-(2-(dimethylamino)ethyl)-4-morpholino-7H-pyrrolo[2,3-h]quinazolin-2-yl)aniline and cyclopropane carbonyl chloride. MS (ESI) m/z

### Example 97

### N-(4-{7-[2-(dimethylamino)ethyl]-4-morpholin-4-yl-7H-pyrrolo[2,3-h]quinazolin-2-yl}phenyl)butanamide

The title compound was prepared by following the procedure described above for example 16, using 4-(7-(2-(dimethylamino)ethyl)-4-morpholino-7H-pyrrolo[2,3-h]quinazolin-2-yl)aniline and butyryl chloride. MS (ESI) m/z

### Example 98

### methyl (4-{7-[2-(dimethylamino)ethyl]-4-morpholin-4-yl-7H-pyrrolo[2,3-h]quinazolin-2-yl}phenyl)carbamate

The title compound was prepared by following the procedure described above for example 2, using 4-(7-(2-(dimethylamino)ethyl)-4-morpholino-7H-pyrrolo[2,3-h]quinazolin-2-yl)aniline, triphosgene and methanol. MS (ESI) m/z

### Example 99

### 1-(1-methylethyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and isopropyl amine. MS (ESI) m/z

### Example 100

### 1-(cyclopropylmethyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and (aminomethyl)cyclopropane. MS (ESI) m/z

### Example 101

### 1-(2-methoxyethyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and methoxyethylamine. MS (ESI) m/z

### Example 102

### 1-[4-(7-methyl-4-morpholin-4-yl-7H-pyrrolo[2,3-h]quinazolin-2-yl)phenyl]-3-(tetrahydrofuran-2-ylmethyl)urea

The title compound was prepared by following the procedure as outlined in Example 2 using 4-(7-methyl-4-morpholin-4-yl-7H-pyrrolo[2,3-h]quinazolin-2-yl)aniline, triphosgene and tetrahydrofurfurylamine. MS (ESI) m/z

### Example 103

### 1-(2-cyclohex-1-en-1-ylethyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 2-(1-cyclohexenyl)ethylamine. MS (ESI) m/z

### Example 104

### 1-[4-(7-methyl-4-morpholin-4-yl-7H-pyrrolo[2,3-h]quinazolin-2-yl)phenyl]-3-(3-pyrrolidin-1-yl]propyl)urea

The title compound was prepared by following the procedure as outlined in Example 2 using 4-(7-methyl-4-morpholin-4-yl-7H-pyrrolo[2,3-h]quinazolin-2-yl)aniline, triphosgene and 1-(3-aminopropyl)pyrrolidine. MS (ESI) m/z

### Example 105

### 1-cyclopentyl-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and cyclopentylamine. MS (ESI) m/z

### Example 106

### 1-cyclobutyl-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and cyclobutylamine. MS (ESI) m/z

### Example 107

### 1-cyclopropyl-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and cyclobutylamine. MS (ESI) m/z

### Example 108

### 1-cyclohexyl-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and cyclohexylamine. MS (ESI) m/z

### Example 109

### propyl {4-[7-(methylsulfonyl)-4-morpholin-4-yl-7H-pyrrolo[2,3-h]quinazolin-2-yl]phenyl}carbamate

The title compound was prepared by following the procedure as outlined in Example 2 using 4-(7-(methylsulfonyl)-4-morpholino-7H-pyrrolo[2,3-h]quinazolin-2-yl)aniline, triphosgene and propyl chloroformate. MS (ESI) m/z

### Example 110

### 1-methyl-3-{4-[7-(methylsulfonyl)-4-morpholin-4-yl-7H-pyrrolo[2,3-h]quinazolin-2-yl]phenyl}urea

The title compound was prepared by following the procedure as outlined in Example 2 using 4-(7-(methylsulfonyl)-4-morpholino-7H-pyrrolo[2,3-h]quinazolin-2-yl)aniline and triphosgene and methylamine. MS (ESI) m/z

### Example 111

### 1-(4-{7-[2-(dimethylamino)ethyl]-4-morpholin-4-yl-7H-pyrrolo[2,3-h]quinazolin-2-yl}phenyl)-3-pyridin-3-ylurea

The title compound was prepared by following the procedure described above for example 2, using 4-(7-(2-(dimethylamino)ethyl)-4-morpholino-7H-pyrrolo[2,3-h]quinazolin-2-yl)aniline, triphosgene and 3-aminopyridine. MS (ESI) m/z

### Example 112

### N-(4-{7-[2-(dimethylamino)ethyl]-4-morpholin-4-yl-7H-pyrrolo[2,3-h]quinazolin-2-yl}phenyl)acetamide

The title compound was prepared by following the procedure described above for example 16, using 4-(7-(2-(dimethylamino)ethyl)-4-morpholino-7H-pyrrolo[2,3-h]quinazolin-2-yl)aniline and acetyl chloride. MS (ESI) m/z

### Example 113

### 1-(4-{7-[2-(dimethylamino)ethyl]-4-morpholin-4-yl-7H-pyrrolo[2,3-h]quinazolin-2-yl}phenyl)-3-methylurea

The title compound was prepared by following the procedure described above for example 2, using 4-(7-(2-(dimethylamino)ethyl)-4-morpholino-7H-pyrrolo[2,3-h]quinazolin-2-yl)aniline, triphosgene and methylamine. MS (ESI) m/z

### Example 114

### 1-(3-acetylphenyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 3′-aminoacetophenone. MS (ESI) m/z

### Example 115

### 1-(4-acetylphenyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 4′-aminoacetophenone. MS (ESI) m/z

### Example 116

### 1-(3,5-dimethylisoxazol-4-yl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 3,5-dimethylisoxazol-4-ylamine. MS (ESI) m/z

### Example 117

### 1-(1,1-dioxidotetrahydrothiophen-3-yl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 1,1-dioxidotetrahydrothiophen-3-ylamine. MS (ESI) m/z

### Example 118

### 1-(2-fluoroethyl)-3-[4-(7-methyl-4-morpholin-4-yl-7H-pyrrolo[2,3-h]quinazolin-2-yl)phenyl]urea

The title compound was prepared by following the procedure as outlined in Example 2 using 4-(7-methyl-4-morpholin-4-yl-7H-pyrrolo[2,3-h]quinazolin-2-yl)aniline, triphosgene and 2-fluoroethylamine. MS (ESI) m/z

### Example 119

### 1-[4-(7-methyl-4-morpholin-4-yl-7H-pyrrolo[2,3-h]quinazolin-2-yl)phenyl]-3-(2,2,2-trifluoroethyl)urea

The title compound was prepared by following the procedure as outlined in Example 2 using 4-(7-methyl-4-morpholin-4-yl-7H-pyrrolo[2,3-h]quinazolin-2-yl)aniline, triphosgene and 2,2,2-trifluoroethylamine. MS (ESI) m/z

### Example 120

### 1-[4-(7-methyl-4-morpholin-4-yl-7H-pyrrolo[2,3-h]quinazolin-2-yl)phenyl]-3-(2-pyridin-4-ylethyl)urea

The title compound was prepared by following the procedure as outlined in Example 2 using 4-(7-methyl-4-morpholin-4-yl-7H-pyrrolo[2,3-h]quinazolin-2-yl)aniline, triphosgene and 4-(2-aminoethyl)pyridine. MS (ESI) m/z

### Example 121

### 1-{4-[7-(methylsulfonyl)-4-morpholin-4-yl-7H-pyrrolo[2,3-h]quinazolin-2-yl]phenyl}-3-propylurea

The title compound was prepared by following the procedure as outlined in Example 2 using 4-(7-(methylsulfonyl)-4-morpholino-7H-pyrrolo[2,3-h]quinazolin-2-yl)aniline, triphosgene and aminopropane. MS (ESI) m/z

### Example 122

### 1-[3-(7-methyl-4-morpholin-4-yl-7H-pyrrolo[2,3-h]quinazolin-2-yl)phenyl]-3-pyridin-4-ylurea

3-(7-methyl-4-morpholino-7H-pyrrolo[2,3-h]quinazolin-2-yl)aniline was prepared as described in example 1 using 2-chloro-7-methyl-4-morpholin-4-yl-7H-pyrrolo[2,3-h]quinazoline and 3-aminophenylboronic acid, pinacol ester.

The title compound was prepared by following the procedure as outlined in Example 2 using 3-(7-methyl-4-morpholino-7H-pyrrolo[2,3-h]quinazolin-2-yl)aniline, triphosgene and 4-aminopyridine. MS (ESI) m/z

### Example 123

### 1-[3-(7-methyl-4-morpholin-4-yl-7H-pyrrolo[2,3-h]quinazolin-2-yl)phenyl]-3-pyridin-3-ylurea

The title compound was prepared by following the procedure as outlined in Example 2 using 3-(7-methyl-4-morpholino-7H-pyrrolo[2,3-h]quinazolin-2-yl)aniline, triphosgene and 3-aminopyridine. MS (ESI) m/z

### Example 124

### ethyl [3-(7-methyl-4-morpholin-4-yl-7H-pyrrolo[2,3-h]quinazolin-2-yl)phenyl]carbamate

The title compound was prepared by following the procedure as outlined in Example 16 using 3-(7-methyl-4-morpholino-7H-pyrrolo[2,3-h]quinazolin-2-yl)aniline and ethyl chloroformate. MS (ESI) m/z

### Example 125

### N-[3-(7-methyl-4-morpholin-4-yl-7H-pyrrolo[2,3-h]quinazolin-2-yl)phenyl]cyclopropanecarboxamide

The title compound was prepared by following the procedure as outlined in Example 216 using 3-(7-methyl-4-morpholino-7H-pyrrolo[2,3-h]quinazolin-2-yl)aniline and cyclopropanecarbonyl chloride. MS (ESI) m/z

### Example 126

### N-[3-(7-methyl-4-morpholin-4-yl-7H-pyrrolo[2,3-h]quinazolin-2-yl)phenyl]butanamide

The title compound was prepared by following the procedure as outlined in Example 16 using 3-(7-methyl-4-morpholino-7H-pyrrolo[2,3-h]quinazolin-2-yl)aniline and butyryl chloride. MS (ESI) m/z

### Example 127

### 1-[3-(7-methyl-4-morpholin-4-yl-7H-pyrrolo[2,3-h]quinazolin-2-yl)phenyl]-3-propylurea

The title compound was prepared by following the procedure as outlined in Example 2 using 3-(7-methyl-4-morpholino-7H-pyrrolo[2,3-h]quinazolin-2-yl)aniline, triphosgene and aminopropane. MS (ESI) m/z

### Example 128

### 1-ethyl-3-[3-(7-methyl-4-morpholin-4-yl-7H-pyrrolo[2,3-h]quinazolin-2-yl)phenyl]urea

The title compound was prepared by following the procedure as outlined in Example 2 using 3-(7-methyl-4-morpholino-7H-pyrrolo[2,3-h]quinazolin-2-yl)aniline, triphosgene and ethylamine. MS (ESI) m/z

### Example 129

### N-[3-(7-methyl-4-morpholin-4-yl-7H-pyrrolo[2,3-h]quinazolin-2-yl)phenyl]propanamide

The title compound was prepared by following the procedure as outlined in Example 16 using 3-(7-methyl-4-morpholino-7H-pyrrolo[2,3-h]quinazolin-2-yl)aniline and propyl chloride. MS (ESI) m/z

### Example 130

### METHYL 4-({[4-(7-ETHYL-4-MORPHOLIN-4-YL-7H-PYRROLO[2,3-H]QUINAZOLIN-2-YL)PHENYL]CARBAMOYL}AMINO)BENZOATE MS (ESI) m/z 551.2.

### Example 131

### 1-[4-(7-ETHYL-4-MORPHOLIN-4-YL-7H-PYRROLO[2,3-H]QUINAZOLIN-2-YL)PHENYL]-3-{4-[(4-METHYLPIPERAZIN-1-YL)CARBONYL]PHENYL}UREA MS (ESI) m/z 618.7.

### Example 132

### 4-({[4-(7-ETHYL-4-MORPHOLIN-4-YL-7H-PYRROLO[2,3-H]QUINAZOLIN-2-YL)PHENYL]CARBAMOYL}AMINO)-N-[2-(METHYLAMINO)ETHYL]BENZAMIDE MS (ESI) m/z 592.7.

### Example 133

### N-[2-(DIMETHYLAMINO)ETHYL]-4-({[4-(7-ETHYL-4-MORPHOLIN-4-YL-7H-PYRROLO[2,3-H]QUINAZOLIN-2-YL)PHENYL]CARBAMOYL}AMINO)-N-METHYLBENZAMIDE MS (ESI) m/z 620.7

### Example 134

### 1-(4-{[4-(DIMETHYLAMINO)PIPERIDIN-1-YL]CARBONYL}PHENYL)-3-[4-(7-ETHYL-4-MORPHOLIN-4-YL-7H-PYRROLO[2,3-H]QUINAZOLIN-2-YL)PHENYL]UREA MS (ESI) m/z 646.7.

### Biological Evaluation

### mTOR Kinase Assay Methods

Human mTOR assays (See Toral-Barza, et al. Biochem Biophys. Res. Commun. Jun. 24, 2005;332(1):304-10) with purified enzyme are performed in 96-well plates by DELFIA format as follows. Enzymes are first diluted in kinase assay buffer (10 mM HEPES (pH 7.4), 50 mM NaCl, 50 mM β-glycerophosphate, 10 mM MnCl₂, 0.5 mM DTT, 0.25 mM microcystin LR, and 100 mg/mL BSA). To each well, 12 μL of the diluted enzyme is mixed briefly with 0.5 μL test inhibitor or control vehicle dimethylsulfoxide (DMSO). The kinase reaction is initiated by adding 12.5 μL kinase assay buffer containing ATP and His6-S6K to give a final reaction volume of 25 μL containing 800 ng/mL FLAG-TOR, 100 mM ATP and 1.25 mM His6-S6K. The reaction plate is incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution can be purchased from PerkinElmer. 45 μL of the terminated kinase reaction mixture is transferred to a MaxiSorp plate (Nunc) containing 55 μL PBS. The His6-S6K is allowed to attach for 2 hours after which the wells are aspirated and washed once with PBS. 100 μL of DELFIA Assay buffer with 40 ng/mL Eu-P(T389)-S6K antibody is added. The antibody binding is continued for 1 hour with gentle agitation. The wells are then aspirated and washed 4 times with PBS containing 0.05% Tween-20 (PBST). 100 μL of DELFIA Enhancement solution is added to each well and the plates are read in a PerkinElmer Victor model plate reader. Data obtained is used to calculate enzymatic activity and enzyme inhibition by potential inhibitors.

### PI3K-Alpha and PI3K-Gamma Fluorescence Polarization Assay Protocols

The reaction buffer was 20 mM HEPES, pH 7.5, 2 mM MgCl₂, 0.05% CHAPS; and 0.01% βME (added fresh). The Stop/Detection Buffer was 100 mM HEPES, pH 7.5, 4 mM EDTA, 0.05% CHAPS; ATP 20 mM in water; PIP2 (diC8, Echelon, Salt Lake City Utah cat #P-4508) 1 mM in water (MW=856.5). The GST-GRP was 1.75 mg/mL or 1.4 mg/mL in 10% glycerol. The Red detector (TAMRA) was 2.5 μM. Nunc 384-well black polypropylene fluorescent plates were used for PI3K assays.

The assay is run by placing 5 μL of diluted enzyme per well, then 5 μL of diluted compound (or 9.5 μL enzyme then 0.5 μL compound in DMSO) is added and mixed. Then, 10 μL substrate is added to start the reaction. The samples are incubated 30-60 minutes, then the reaction is stopped by adding 20 μL stop/detector mix. PI3K is diluted with reaction buffer (e.g., 5 μL or 7.5 μL PI3K into 620 μL reaction buffer), and 5 μL of diluted enzyme is used per well. A 5 μL portion of reaction buffer or of drug diluted in buffer (e.g., 4 μL/100 so final DMSO is 1% in reaction) is added to each. Pipetting up and down mixes the samples. Alternatively, the enzyme can be diluted to 1215 μL. In this case 9.8 μL is added per well and 0.2 μL compound is added in DMSO.

To prepare 1 mL of substrate solution, 955 μL reaction buffer, 40 μL PIP2, and 2.5 μL ATP are mixed. 10 μL of substrate is added to each well to start the reaction. This results in 20 μM PIP2, and 25 μM ATP per reaction. The stop/detector mix is prepared by mixing 4 μL Red detector and 1.6 μL or 2.0 μL GST-GRP with 1 mL stop buffer, which results in 10 nM probe and 70 nM GST-GRP. 20 μL of the stop/detector mix is added to each well to stop the reaction. The plates are read after 30-90 minutes keeping the red probe solutions dark. For the zero time point, stop/detector mix is added to the enzyme just before adding substrate. For an extra control, stop/detector mix is added to buffer (no enzyme) and substrate or to just buffer (no substrate). Pooled PI3K preparations had a protein concentration of 0.25 mg/mL. The recommended reaction has 0.06 μL per 20 μL (0.015 μg/20 μL) or 0.01125 μg/15 μL or 0.75 μg/mL.

Plates are read on machines with filters for TAMRA. The units are mP with no enzyme controls reading app 190-220 mP units. Fully active enzyme reduces fluorescence polarization down to 70-100 mP after 30 minutes. An active compound raises the mP values halfway to control or to 120-150 mP units. Compounds of the invention had IC₅₀s against PI3K-alpha ranging from 22 nM to 12,000 nM.

### In Vitro Cell Culture Growth Assay Methods

Cell Lines used are human prostate LNCap and human breast MDA468 tumor cell lines. Cells are plated in 96-well culture plates at approximately 3000 cells per well. One day following plating, various concentrations of PI3K inhibitors in DMSO are added to cells (final DMSO concentration in cell assays is 0.25%). Three days after drug treatment, viable cell densities are determined by cell mediated metabolic conversion of the dye MTS, a well-established indicator of cell proliferation in vitro. Cell growth assays are performed using kits purchased from Promega Corporation (Madison, Wis.), following the protocol provided by the vendor. Measuring absorbance at 490 nm generates MTS assay results. Compound effect on cell proliferation is assessed relative to untreated control cell growth. The drug concentration that conferred 50% inhibition of growth is determined as IC₅₀ (μM).

Several compounds were tested in the above mentioned cell based assay and they found to have IC₅₀ values in the range of 0.048 μM to 30 μM.

### hSMG-1 Kinase Assay

The human SMG-1 (hSMG-1) kinase assay employs the recombinant hSMG-1 protein prepared from transiently transfected HEK293 cells and a GST-p53 (aa 1-70) fusion substrate protein derived from cellular tumor suppressor gene p53. The routine assay is performed in a 96-well plate format as follows. Enzymes were first diluted in kinase assay buffer (10 mM HEPES, pH 7.4, 50 mM NaCl, 0.2 mM DTT, 50 mM β-glycerophosphate, 0.5 μM microcystin LR, 10 mM MnCl₂). To each well, 12 μL of the diluted enzyme were mixed briefly with 0.5 μL test inhibitor or control vehicle dimethylsulfoxide (DMSO). The kinase reaction was initiated by adding 12.5 μL kinase assay buffer containing ATP and GST-p53 to give a final reaction volume of 25 μL containing 400-800 ng/mL FLAG-hSMG-1, 0.5 μg GST-p53, 10 μM ATP. The reaction was carried out at room temperature for 1.0 hour before terminated by addition of 25 μl stop solution. The assay mixture was then transferred to FluoroNunc Plates with MaxiSorp Surface (Nunc #439454). The plates were incubated at room temperature for 2 hr (4° C. for overnight) to achieve efficient binding of substrate protein to the plate. The plates were aspirated, washed with PBS. Phospho-substrate proteins were detected by incubating for 1 hour with 125 ng of europium-labeled anti-mouse secondary antibody (PerkinElmer AD2027) and the primary phospho(S15)-p53 monoclonal antibody (Cell Signal #9286) in 100 μL DELFIA assay buffer (PerkinElmer #1244-111). Plates were then washed and incubated for 0.5 hour with 100 μl of DELFIA enhancement solution (PerkinElmer #1244-105). DELFIA assay results are recorded in a Victor Plate Reader (PerkinElmer). Data obtained were used to calculate enzymatic activity and enzyme inhibition by potential inhibitors.

Table 1 shows the results of the described biological assays.

TABLE 1

| | TOR | PI3 | PI3 | | | |
|---|---|---|---|---|---|---|
| | Kinase | Kinase α | Kinase γ | LNCap | MDA468 | hSMG1 |
| Example | IC₅₀ (μM) | IC₅₀ (nM) | IC₅₀ (nM) | IC₅₀ (μM) | IC₅₀ (μM) | IC₅₀ (μM) |
| 1 | 0.805 | 6482 | 10000 | | | |
| 2 | 0.066 | 428 | 5389 | | | |
| 3 | 0.035 | 2109 | 4038 | | | |
| 4 | 2.05 | 728 | 2835 | | | |
| 5 | 2.05 | 2700 | 10000 | | | |
| 6 | 2.55 | 7627 | 10000 | | | |
| 7 | 0.56 | 1690 | 3502 | | | |
| 8 | 0.19 | 431 | 3035 | | | |
| 9 | 0.075 | 2700 | 5129 | | | |
| 10 | 0.021 | 71 | 247 | | | |
| 11 | 0.0037 | 101 | 437 | | | |
| 12 | 0.355 | 2000 | 1328 | | | |
| 13 | 0.26 | 62 | 234 | | | |
| 14 | 0.125 | 239 | 634 | | | |
| 15 | 2.15 | 2523 | 3312 | | | |
| 16 | 0.37 | 3082 | >10,000 | | | |
| 17 | 1.4 | >10,000 | 9853 | | | |
| 18 | 1.45 | 4460 | 5620 | | | |
| 19 | 1.25 | 12000 | 6166 | | | |
| 20 | 0.39 | 3314 | 2094 | | | |
| 21 | 1.2 | 7830 | >10,000 | | | |
| 22 | 5.3 | 8396 | 6159 | | | |
| 23 | 0.32 | 5326 | 9500 | | | |
| 24 | 1.65 | 2347 | >10,000 | | | |
| 25 | 3.05 | 7550 | >10,000 | | | |
| 26 | 2.1 | 5422 | >10,000 | | | |
| 27 | 2.8 | 6586 | 6656 | | | |
| 28 | 2.7 | 2506 | 4524 | | | |
| 29 | 3.8 | 2969 | 4074 | | | |
| 30 | 9.15 | 455 | 4951 | | | |
| 31 | >20 | 4033 | 2484 | | | |
| 32 | 4.55 | 322 | 3622 | | | |
| 33 | 0.015 | 222 | 862 | | | |
| 34 | 1.3 | 201 | 3191 | | | |
| 35 | 0.41 | 174 | 2232 | | | |
| 36 | 1.6 | 1759 | 4000 | | | |
| 37 | 1.2 | 251 | 3197 | | | |
| 38 | 4 | 12000 | 9838 | | | |
| 39 | 1.72 | 2564 | 7415 | | | |
| 40 | 2.3 | 4278 | 2766 | | | |
| 41 | 1.45 | >10,000 | >10,000 | | | |
| 42 | 0.63 | 616 | 3586 | | | |
| 43 | 0.247 | 48 | 426 | | | |
| 44 | 4 | 9364 | 9500 | | | |
| 45 | 1.13 | 2464 | 2829 | | | |
| 46 | 0.42 | 216 | 209 | | | |
| 47 | 2.05 | >10,000 | >10,000 | | | |
| 48 | 7.55 | >10,000 | >10,000 | | | |
| 49 | 0.34 | 6306 | 4662 | | | |
| 50 | 2.35 | 668 | >10,000 | | | |
| 51 | >20,000 | >10,000 | >10,000 | | | |
| 52 | 1.2 | 8885 | 3665 | | | |
| 53 | 0.91 | 214 | 1007 | | | |
| 54 | 0.021 | 5659 | 8400 | | | |
| 55 | 0.525 | 4341 | 6163 | | | |
| 56 | 0.05 | 6970 | 6698 | | | |
| 57 | 0.235 | 514 | 3584 | | | |
| 58 | 0.155 | 102 | 471 | | | |
| 59 | 2.6 | 63 | 560 | | | |
| 60 | 1.35 | 775 | 4894 | | | |
| 61 | 0.13 | 384 | 3491 | | | |
| 62 | 0.028 | 114 | 519 | | | |
| 63 | 0.067 | 119 | 491 | | | |
| 64 | 0.044 | 74 | 313 | | | |
| 65 | 0.073 | 48 | 300 | | | |
| 66 | 2.25 | 245 | 5752 | | | |
| 67 | 0.67 | 231 | 1578 | | | |
| 68 | NA | NA | NA | | | |
| 69 | 1.11 | 274 | 1130 | | | |
| 70 | 5.8 | 168 | 2067 | | | |
| 71 | 0.545 | 52 | 114 | | | |
| 72 | 3.25 | 4007 | 5162 | | | |
| 73 | 0.063 | 62 | 198 | | | |
| 74 | 0.545 | 212 | 1187 | | | |
| 75 | 16.5 | >10,000 | >10,000 | | | |
| 76 | 0.13 | 65 | 82 | | | |
| 77 | 0.525 | 451 | 933 | | | |
| 78 | 0.305 | 73 | 390 | | | |
| 79 | 0.435 | 1083 | 6086 | | | |
| 80 | 2.075 | 10314 | >10,000 | | | |
| 81 | NA | NA | NA | | | |
| 82 | 0.016 | 111 | 147 | | | |
| 83 | 1.7 | 123 | 320 | | | |
| 84 | | | | | | 0.475 |
| 85 | | 22 | 142 | 0.048 | 0.070 | 0.160 |
| 86 | | | | | | 15.500 |
| 87 | | | | | | 5.600 |
| 88 | | | | | | >20.000 |
| 89 | | | | 0.690 | 1.900 | 0.200 |
| 90 | | | | | | 5.600 |
| 91 | | | | | | 6.100 |
| 92 | | | | | | 5.250 |
| 93 | | | | | | >20.000 |
| 94 | | | | | | 2.000 |
| 95 | 0.050 | | | | | 1.800 |
| 96 | 5.250 | | | | | >20.000 |
| 97 | 6.200 | | | | | >20.000 |
| 98 | 2.000 | | | | | >20.000 |
| 99 | 0.215 | | | | | 14.500 |
| 100 | 0.500 | | | | | 16.000 |
| 101 | 1.800 | | | | | >20.000 |
| 102 | 1.500 | | | | | >17.000 |
| 103 | 13.750 | | | | | >20.000 |
| 104 | 2.400 | | | | | >20.000 |
| 105 | 2.050 | | | | | >20.000 |
| 106 | 1.500 | | | | | 16.500 |
| 107 | 0.545 | | | | | >14.000 |
| 108 | 7.250 | | | | | >20.000 |
| 109 | 2.050 | | | | | >20.000 |
| 110 | 0.002 | 95 | 1910 | 0.880 | 1.700 | 0.043 |
| 111 | 0.015 | 118 | 860 | | | 0.290 |
| 112 | 2.800 | | | | | >20.000 |
| 113 | 0.067 | 427 | 3090 | | | 0.490 |
| 114 | 5.550 | | | | | 10.500 |
| 115 | 0.470 | | | | | 4.250 |
| 116 | 1.275 | | | | | >20.000 |
| 117 | 0.600 | | | | | >20.000 |
| 118 | 0.625 | | | | | 15.000 |
| 119 | 1.260 | | | | | 6.500 |
| 120 | 0.625 | | | | | 3.300 |
| 121 | 0.026 | | | 2.000 | 30.000 | 0.140 |
| 122 | 1.200 | | | | | 6.500 |
| 123 | 2.550 | | | | | 5.500 |
| 124 | 1.900 | | | | | >20.000 |
| 125 | 1.300 | | | | | >20.000 |
| 126 | 1.900 | | | | | >20.000 |
| 127 | 1.500 | | | | | 15.500 |
| 128 | 1.800 | | | | | >20.000 |
| 129 | 1.500 | | | | | >20.000 |

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Throughout this application, various publications are referenced. The disclosures of these publications in their entireties are hereby incorporated by reference into this application in order to more fully describe the state of the art as known to those skilled therein as of the date of the invention described and claimed herein.

The compounds within the present invention possess double bonds connecting the indole to the benzofuran or benzothiophene nucleus. These double bonds can exist as geometric isomers, and the invention includes both E and Z isomers of such double bonds. All such stable isomers are contemplated in the present invention.

What is claimed:

1. A compound of the Formula I: 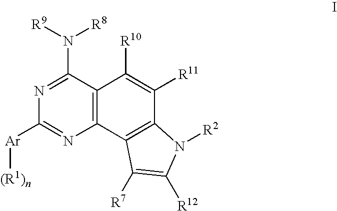
or pharmaceutically acceptable salt thereof, wherein
Ar is phenyl, naphthyl, or nitrogen-containing mono- or bicyclic C₁-C₉heteroaryl;
R¹ is independently NR³R⁴; NHC(O)NR³R⁴; —NHC(O)OR⁵; R⁵C(O)NH—; R⁵C(O)—; R⁵S(O)ₚNH—; CHO; C₁-C₆hydroxylalkyl-; C₃-C₆hydroxylalkenyl-; (C₆-C₁₄aryl)alkyl optionally substituted by hydroxyl; (C₆-C₁₄aryl)alkyl-O—; (C₁-C₆alkoxy)carbonyl; HO₂C—; R³R⁴NC(O)—; N≡C—; carboxyamido(C₁-C₆)alkyl-; hydroxyl; halo; C₁-C₆alkoxy optionally substituted with from 1 to 3 substituents independently selected from C₁-C₆alkoxy, —NH₂, —NH(C₁-C₆alkyl), and —N(C₁-C₆alkyl)(C₁-C₆alkyl); —NH(SO₂)NH—(C₁-C₆alkyl); —NH(SO₂)N—(C₁-C₆alkyl)(C₁-C₆alkyl); —O-heterocycle optionally substituted by C₁-C₆alkyl; H₂NC₁-C₆alkyleneSO₂—; (C₁-C₆alkyl)NHC₁-C₆alkyleneSO₂—; (C₁-C₆alkyl)(C₁-C₆alkyl)NC₁-C₆alkyleneSO₂—; heterocyclyl(C₁-C₆alkyl)SO₂—; carboxyamido(C₁-C₆)alkyl-C(O)—; heterocycle-C(O)—C₁-C₆alkylene-C(O)—; R³R⁴NSO₂C₁-C₆alkylene-C(O)—; or —SO₂NR³R⁴;
n is 0, 1, 2, 3, 4, or 5;
each p is independently 1 or 2;
R³ and R⁴ are each independently:
(a). H;
(b). C₁-C₆alkyl optionally substituted with from 1 to 3 substituents independently selected from:
(i). —NH₂,
(ii). —NH(C₁-C₆alkyl),
(iii). —N(C₁-C₆alkyl)(C₁-C₆alkyl),
(iv). C₁-C₆alkoxy,
(v). C₃-C₈cycloalkyl,
(vi). C₃-C₈cycloalkenyl,
(vii). halo,
(viii). and C₁-C₉heteroaryl;
(c). C₁-C₉heteroaryl optionally substituted with from 1 to 3 substituents independently selected from:
(i). C₁-C₆alkyl,
(ii). C₁-C₆aminoalkyl-,
(iii). C₁-C₆hydroxylalkyl-,
(iv). and C₁-C₉heterocyclyl-;
(d). heterocyclyl(C₁-C₆alkyl)-;
(e). (C₁-C₉heterocyclyl)-;
(f). (C₁-C₉heterocyclyl)-SO₂—;
(g). C₆-C₁₄aryl optionally substituted with from 1 to 3 substituents independently selected from:
(i). C₁-C₆alkyl,
(ii). C₁-C₆alkoxy,
(iii). C₁-C₆aminoalkyl-,
(iv). C₁-C₆hydroxylalkyl-,
(v). C₁-C₆aminoalkyl-NH—,
(vi). C₁-C₆hydroxylalkyl-NH—,
(vii). halo,
(viii). C₁-C₉heterocyclyl,
(ix). (C₁-C₉heteroaryl)-O—,
(x). —(C₁-C₉heterocycle)-O—,
(xi). (C₁-C₉heterocyclyl)-S—,
(xii). (C₁-C₉heterocyclyl)-CO—,
(xiii). (C₁-C₆alkyl)-NH—C(O)—,
(xiv). (C₁-C₆alkyl)(C₁-C₆alkyl)N—C(O)—,
(xv). H₂NNH—C(O)—,
(xvi). R⁵—C(O)—,
(xvii). (C₁-C₆alkyl)-NH—NH—C(O)—,
(xviii). (C₁-C₆alkyl)(C₁-C₆alkyl)NNH—C(O)—,
(xix). (C₁-C₉heteroaryl)NH—C(O)—,
(xx). (C₆-C₁₄aryl)NH—C(O)—,
(xxi). (C₁-C₆alkyl)-SO₂—,
(xxii). (C₁-C₉heterocyclyl)-SO₂—,
(xxiii). H₂NS(O)₂—,
(xxiv). (C₁-C₆alkyl)NH—SO₂—,
(xxv). (C₁-C₆alkyl)(C₁-C₆alkyl)N—SO₂—,
(xxvi). H₂NNHS(O)₂—,
(xxvii). (C₁-C₆alkyl)NH—NH—SO₂—,
(xxviii). (C₁-C₆alkyl)(C₁-C₆alkyl)N—NH—SO₂—,
(xxix). (C₁-C₉heteroaryl)NH—S(O)₂—,
(xxx). (C₆-C₁₄aryl)NH—S(O)₂—,
(xxxi). and perfluoro(C₁-C₆)alkyl-;
(h). or C₃-C₈cycloalkyl-;
or R³ and R⁴, when taken together with the nitrogen to which they are attached, can form a 3- to 7-membered nitrogen containing heterocycle wherein up to two of the carbon atoms of the heterocycle can be replaced with —N(R⁶)—, —O—, S, S(O), or —S(O)₂—;
R⁶ is hydrogen or C₁-C₆alkyl;
R⁵ is C₁-C₆alkyl-, C₃-C₈cycloalkyl-, C₁-C₉heteroaryl, or C₆-C₁₄aryl- optionally substituted with from 1 to 3 substituents independently selected halogens;
R² is H; C₁-C₆alkyl optionally substituted with from 1 to 3 substituents independently selected from —NH₂, —NH(C₁-C₆alkyl), —N(C₁-C₆alkyl)(C₁-C₆alkyl), C₆-C₁₄aryl, and —C(O)O(C₁-C₆alkyl); —S(O)_{q}—(C₁-C₆alkyl); —S(O)_{q}—(C₁-C₉heteroaryl); —S(O)_{q}—(C₆-C₁₄aryl); or —S(O)_{q}-(4- to 7-membered monocyclic heterocycle group) optionally substituted with from 1 to 3 substituents independently selected from C₁-C₆alkyl and (C₆-C₁₄aryl)alkyl-O—C(O)—;
q is independently 1 or 2;
R⁷ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₂-C₁₀alkenyl; C₂-C₁₀alkynyl; halo; C₁-C₉heteroaryl; C₆-C₁₄aryl optionally substituted with from 1 to 3 substituents independently selected from C₁-C₆alkyl, halo, and perfluoro(C₁-C₆)alkyl; C₃-C₈cycloalkyl; or CHO;
R⁸ and R⁹ are each independently H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
or R⁸ and R⁹, when taken together with the nitrogen to which they are attached, can form a 3- to 7-membered nitrogen containing heterocycle wherein up to two of the carbon atoms of the heterocycle can be replaced with —N(R¹³)—, —O—, S, S(O), or —S(O)₂;
R¹³ is hydrogen or C₁-C₆alkyl;
R¹⁰ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
R¹¹ is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl;
R¹² is H; C₁-C₆alkyl optionally substituted with from 1 to 3 substituents independently selected from —NH₂, —NH(C₁-C₆alkyl), —N(C₁-C₆alkyl)(C₁-C₆alkyl), and C₁-C₉heteroaryl; C₁-C₉heteroaryl; C₆-C₁₄aryl optionally substituted with from 1 to 3 substituents independently selected from C₁-C₆alkyl, halo, and perfluoro(C₁-C₆)alkyl; or C₃-C₈cycloalkyl.

2. A compound of claim 1 of the Formula II: 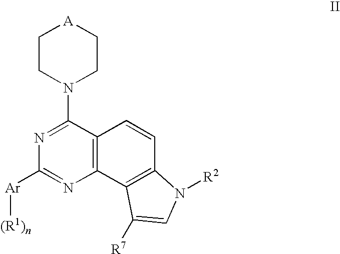
wherein
A is —O—, —CH₂O—, or —S(O)ₘ—;
m is 0, 1, or 2;
and the remaining variables are as defined in claim 1.

3. A compound of claim 1 or claim 2 wherein, n is 1.

4. A compound of claim 2 wherein, A is —O—.

5. A compound of claim 2 wherein, R¹ is —NHC(O)NR³R⁴.

6. A compound of claim 5 wherein, R³ is C₁-C₆alkyl, C₁-C₉heteroaryl, or C₆-C₁₄aryl.

7. A compound of claim 6 wherein, R³ is methyl or 4-pyridyl.

8. A compound of claim 5 wherein, R⁴ is H.

9. A compound of claim 2 wherein, R² is C₁-C₆alkyl or S(O)_{q}—(C₁-C₆alkyl).

10. A compound of claim 9 wherein, R² is methyl or —SO₂—CH₃.

11. A compound of claim 2 wherein, R⁷ is H.

12. A compound selected from the group consisting of:
4-(7-methyl-4-morpholin-4-yl-7H-pyrrolo[2,3-h]quinazolin-2-yl)aniline;
1-methyl-3-[4-(7-methyl-4-morpholin-4-yl-7H-pyrrolo[2,3-h]quinazolin-2-yl)phenyl]urea;
1-ethyl-3-[4-(7-methyl-4-morpholin-4-yl-7H-pyrrolo[2,3-h]quinazolin-2-yl)phenyl]urea;
1-[2-(dimethylamino)ethyl]-3-[4-(7-methyl-4-morpholin-4-yl-7H-pyrrolo[2,3-h]quinazolin-2-yl)phenyl]urea;
1-[3-(dimethylamino)propyl]-3-[4-(7-methyl-4-morpholin-4-yl-7H-pyrrolo[2,3-h]quinazolin-2-yl)phenyl]urea;
4-methyl-N-[4-(7-methyl-4-morpholin-4-yl-7H-pyrrolo[2,3-h]quinazolin-2-yl)phenyl]piperazine-1-carboxamide;
1-(2-furylmethyl)-3-[4-(7-methyl-4-morpholin-4-yl-7H-pyrrolo[2,3-h]quinazolin-2-yl)phenyl]urea;
1-[3-(1H-imidazol-1-yl)propyl]-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-pyridin-2-ylurea;
1-[4-(7-methyl-4-morpholin-4-yl-7H-pyrrolo[2,3-h]quinazolin-2-yl)phenyl]-3-phenylurea;
1-[4-(7-methyl-4-morpholin-4-yl-7H-pyrrolo[2,3-h]quinazolin-2-yl)phenyl]-3-pyridin-4-ylurea;
1-(4-isopropylphenyl)-3-[4-(7-methyl-4-morpholin-4-yl-7H-pyrrolo[2,3-h]quinazolin-2-yl)phenyl]urea;
1-(3-chlorophenyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-[4-(trifluoromethyl)phenyl]urea;
1-[4-(7-methyl-4-morpholin-4-yl-7H-pyrrolo[2,3-h]quinazolin-2-yl)phenyl]-3-(pyridin-2-ylmethyl)urea;
N-[4-(7-methyl-4-morpholin-4-yl-7H-pyrrolo[2,3-h]quinazolin-2-yl)phenyl]acetamide;
N-[4-(7-methyl-4-morpholin-4-yl-7H-pyrrolo[2,3-h]quinazolin-2-Myl)phenyl]nicotinamide;
N-[4-(7-methyl-4-morpholin-4-yl-7H-pyrrolo[2,3-h]quinazolin-2-yl)phenyl]isonicotinamide;
4-fluoro-N-[4-(7-methyl-4-morpholin-4-yl-7H-pyrrolo[2,3-h]quinazolin-2-yl)phenyl]benzamide;
ethyl [4-(7-methyl-4-morpholin-4-yl-7H-pyrrolo[2,3-h]quinazolin-2-yl)phenyl]carbamate;
N-[4-(7-methyl-4-morpholin-4-yl-7H-pyrrolo[2,3-h]quinazolin-2-yl)phenyl]methanesulfonamide;
N-[4-(7-methyl-4-morpholin-4-yl-7H-pyrrolo[2,3-h]quinazolin-2-yl)phenyl]benzenesulfonamide;
3-(7-methyl-4-morpholin-4-yl-7H-pyrrolo[2,3-h]quinazolin-2-yl)benzaldehyde;
1-[3-(7-methyl-4-morpholin-4-yl-7H-pyrrolo[2,3-h]quinazolin-2-yl)phenyl]ethanol;
1-[3-(7-methyl-4-morpholin-4-yl-7H-pyrrolo[2,3-h]quinazolin-2-yl)phenyl]propan-1-ol;
1-[3-(7-methyl-4-morpholin-4-yl-7H-pyrrolo[2,3-h]quinazolin-2-yl)phenyl]prop-2-en-1-ol;
1-[3-(7-methyl-4-morpholin-4-yl-7H-pyrrolo[2,3-h]quinazolin-2-yl)phenyl]but-3-en-1-ol;
3-methyl-1-[3-(7-methyl-4-morpholin-4-yl-7H-pyrrolo[2,3-h]quinazolin-2-yl)phenyl]butan-1-ol;
[3-(7-methyl-4-morpholin-4-yl-7H-pyrrolo[2,3-h]quinazolin-2-yl)phenyl](phenyl)methanol;
(3-{7-[2-(dimethylamino)ethyl]-4-morpholin-4-yl-7H-pyrrolo[2,3-h]quinazolin-2-yl}phenyl)methanol;
2-{2-[3-(benzyloxy)phenyl]-4-morpholin-4-yl-7H-pyrrolo[2,3-h]quinazolin-7-yl}-N,N-dimethylethanamine;
3-{7-[2-(dimethylamino)ethyl]-4-morpholin-4-yl-7H-pyrrolo[2,3-h]quinazolin-2-yl}phenol;
1-(4-{7-[2-(dimethylamino)ethyl]-4-morpholin-4-yl-7H-pyrrolo[2,3-h]quinazolin-2-yl}phenyl)-3-pyridin-4-ylurea;
5-{7-[2-(dimethylamino)ethyl]-4-morpholin-4-yl-7H-pyrrolo[2,3-h]quinazolin-2-yl}pyrimidin-2-amine;
3-(7-methyl-4-morpholin-4-yl-7H-pyrrolo[2,3-h]quinazolin-2-yl)phenol;
Methyl 4-(7-methyl-4-morpholin-4-yl-7H-pyrrolo[2,3-h]quinazolin-2-yl)benzoate;
3-(7-methyl-4-morpholin-4-yl-7H-pyrrolo[2,3-h]quinazolin-2-yl)phenyl]methanol;
4-(7-methyl-4-morpholin-4-yl-7H-pyrrolo[2,3-h]quinazolin-2-yl)benzoic acid;
3-(7-methyl-4-morpholin-4-yl-7H-pyrrolo[2,3-h]quinazolin-2-yl)benzamide;
3-(7-methyl-4-morpholin-4-yl-7H-pyrrolo[2,3-h]quinazolin-2-yl)benzonitrile;
3-(7-methyl-4-morpholin-4-yl-7H-pyrrolo[2,3-h]quinazolin-2-yl)aniline;
5-(7-methyl-4-morpholin-4-yl-7H-pyrrolo[2,3-h]quinazolin-2-yl)pyridin-2-amine;
5-(7-methyl-4-morpholin-4-yl-7H-pyrrolo[2,3-h]quinazolin-2-yl)pyrimidin-2-amine;
N-[3-(7-methyl-4-morpholin-4-yl-7H-pyrrolo[2,3-h]quinazolin-2-yl)benzyl]acetamide;
2-(1H-indol-4-yl)-7-methyl-4-morpholin-4-yl-7H-pyrrolo[2,3-h]quinazoline;
3-(7-benzyl-4-morpholin-4-yl-7H-pyrrolo[2,3-h]quinazolin-2-yl)phenol;
2-(6-methoxypyridin-3-yl)-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyridin-2-ol;
2-(1H-indol-4-yl)-7-methyl-4-morpholin-5-yl-7H-pyrrolo[2,3-h]quinazoline;
2-(2-methoxypyrimidin-5-yl)-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyrimidin-2-ol;
2-(3-fluorophenyl)-7-methyl-4-morpholin-4-yl-7H-pyrrolo[2,3-h]quinazoline;
4-chloro-3-(7-methyl-4-morpholin-4-yl-7H-pyrrolo[2,3-h]quinazolin-2-yl)phenol;
1-[4-(7-methyl-4-morpholin-4-yl-7H-pyrrolo[2,3-h]quinazolin-2-yl)phenyl]-3-propylurea;
N,N-dimethyl-N′-[3-(7-methyl-4-morpholin-4-yl-7H-pyrrolo[2,3-h]quinazolin-2-yl)phenyl]sulfamide;
N-cyclopropyl-3-(7-methyl-4-morpholin-4-yl-7H-pyrrolo[2,3-h]quinazolin-2-yl)benzenesulfonamide;
3-(4-morpholin-4-yl-7H-pyrrolo[2,3-h]quinazolin-2-yl)phenyl]methanol;
-(4-morpholin-4-yl-7H-pyrrolo[2,3-h]quinazolin-2-yl)pyrimidin-2-amine;
3-[7-(methylsulfonyl)-4-morpholin-4-yl-7H-pyrrolo[2,3-h]quinazolin-2-yl]phenol;
tert-butyl [2-(6-aminopyridin-3-yl)-4-morpholin-4-yl-7H-pyrrolo[2,3-h]quinazolin-7-yl]acetate;
benzyl 4-[(4-morpholin-4-yl-2-{4-[(pyridin-4-ylcarbamoyl)amino]phenyl}-7H-pyrrolo[2,3-h]quinazolin-7-yl)sulfonyl]piperidine-1-carboxylate;
1-{4-[4-morpholin-4-yl-7-(piperidin-4-ylsulfonyl)-7H-pyrrolo[2,3-h]quinazolin-2-yl]phenyl}-3-pyridin-4-ylurea;
1-(4-{7-[(1-methylpiperidin-4-yl)sulfonyl]-4-morpholin-4-yl-7H-pyrrolo[2,3-h]quinazolin-2-yl}phenyl)-3-pyridin-4-ylurea;
1-(4-{7-[(1-ethylpiperidin-4-yl)sulfonyl]-4-morpholin-4-yl-7H-pyrrolo[2,3-h]quinazolin-2-yl}phenyl)-3-pyridin-4-ylurea;
1-(4-{7-[(1-isopropylpiperidin-4-yl)sulfonyl]-4-morpholin-4-yl-7H-pyrrolo[2,3-h]quinazolin-2-yl}phenyl)-3-pyridin-4-ylurea;
benzyl 4-{[2-(3-hydroxyphenyl)-4-morpholin-4-yl-7H-pyrrolo[2,3-h]quinazolin-7-yl]sulfonyl}piperidine-1-carboxylate;
3-[4-morpholin-4-yl-7-(piperidin-4-ylsulfonyl)-7H-pyrrolo[2,3-h]quinazolin-2-yl]phenol;
2-[3-(benzyloxy)phenyl]-4-morpholin-4-yl-7-(phenylsulfonyl)-7H-pyrrolo[2,3-h]quinazoline;
3-[4-morpholin-4-yl-7-(phenylsulfonyl)-7H-pyrrolo[2,3-h]quinazolin-2-yl]phenol;
{3-[4-morpholin-4-yl-7-(phenylsulfonyl)-7H-pyrrolo[2,3-h]quinazolin-2-yl]phenyl}methanol;
5-[4-morpholin-4-yl-7-(phenylsulfonyl)-7H-pyrrolo[2,3-h]quinazolin-2-yl]pyrimidin-2-amine;
2-(1H-indazol-4-yl)-4-morpholin-4-yl-7-(phenylsulfonyl)-7H-pyrrolo[2,3-h]quinazoline;
5-[7-(methylsulfonyl)-4-morpholin-4-yl-7H-pyrrolo[2,3-h]quinazolin-2-yl]pyrimidin-2-amine;
{3-[7-(methylsulfonyl)-4-morpholin-4-yl-7H-pyrrolo[2,3-h]quinazolin-2-yl]phenyl}methanol;
2-[5-(methoxymethoxy)pyridin-3-yl]-7-(methylsulfonyl)-4-morpholin-4-yl-7H-pyrrolo[2,3-h]quinazoline;
5-[7-(methylsulfonyl)-4-morpholin-4-yl-7H-pyrrolo[2,3-h]quinazolin-2-yl]pyridin-3-ol;
2-[5-(methoxymethoxy)pyridin-3-yl]-7-methyl-4-morpholin-4-yl-7H-pyrrolo[2,3-h]quinazoline;
5-(7-methyl-4-morpholin-4-yl-7H-pyrrolo[2,3-h]quinazolin-2-yl)pyridin-3-ol;
2-(1H-indazol-4-yl)-7-methyl-4-morpholin-4-yl-7H-pyrrolo[2,3-h]quinazoline;
2-(1H-indazol-4-yl)-4-morpholin-4-yl-7H-pyrrolo[2,3-h]quinazoline;
2-[3-(benzyloxy)phenyl]-4-morpholin-4-yl-7H-pyrrolo[2,3-h]quinazoline;
2-(3-hydroxyphenyl)-4-morpholin-4-yl-7H-pyrrolo[2,3-h]quinazoline-9-carbaldehyde;
[3-(7-benzyl-4-morpholin-4-yl-7H-pyrrolo[2,3-h]quinazolin-2-yl)phenyl]methanol;
1-{4-[7-(methylsulfonyl)-4-morpholin-4-yl-7H-pyrrolo[2,3-h]quinazolin-2-yl]phenyl}-3-phenylurea;
1-{4-[7-(methylsulfonyl)-4-morpholin-4-yl-7H-pyrrolo[2,3-h]quinazolin-2-yl]phenyl}-3-pyridin-3-ylurea;
ethyl {4-[7-(methylsulfonyl)-4-morpholin-4-yl-7H-pyrrolo[2,3-h]quinazolin-2-yl]phenyl}carbamate;
N-{4-[7-(methylsulfonyl)-4-morpholin-4-yl-7H-pyrrolo[2,3-h]quinazolin-2-yl]phenyl}cyclopropanecarboxamide;
N-{4-[7-(methylsulfonyl)-4-morpholin-4-yl-7H-pyrrolo[2,3-h]quinazolin-2-yl]phenyl}butanamide;
1-ethyl-3-{4-[7-(methylsulfonyl)-4-morpholin-4-yl-7H-pyrrolo[2,3-h]quinazolin-2-yl]phenyl}urea;
methyl {4-[7-(methylsulfonyl)-4-morpholin-4-yl-7H-pyrrolo[2,3-h]quinazolin-2-yl]phenyl}carbamate;
N-{4-[7-(methylsulfonyl)-4-morpholin-4-yl-7H-pyrrolo[2,3-h]quinazolin-2-yl]phenyl}propanamide;
N-{4-[7-(methylsulfonyl)-4-morpholin-4-yl-7H-pyrrolo[2,3-h]quinazolin-2-yl]phenyl}acetamide;
ethyl (4-{7-[2-(dimethylamino)ethyl]-4-morpholin-4-yl-7H-pyrrolo[2,3-h]quinazolin-2-yl}phenyl)carbamate;
1-(4-{7-[2-(dimethylamino)ethyl]-4-morpholin-4-yl-7H-pyrrolo[2,3-h]quinazolin-2-yl}phenyl)-3-ethylurea;
1-(4-{7-[2-(dimethylamino)ethyl]-4-morpholin-4-yl-7H-pyrrolo[2,3-h]quinazolin-2-yl}phenyl)-3-phenylurea;
N-(4-{7-[2-(dimethylamino)ethyl]-4-morpholin-4-yl-7H-pyrrolo[2,3-h]quinazolin-2-yl}phenyl)cyclopropanecarboxamide;
N-(4-{7-[2-(dimethylamino)ethyl]-4-morpholin-4-yl-7H-pyrrolo[2,3-h]quinazolin-2-yl}phenyl)butanamide;
methyl (4-{7-[2-(dimethylamino)ethyl]-4-morpholin-4-yl-7H-pyrrolo[2,3-h]quinazolin-2-yl}phenyl)carbamate;
1-(1-methylethyl)-3-[4-(7-methyl-4-morpholin-4-yl-7H-pyrrolo[2,3-h]quinazolin-2-yl)phenyl]urea;
1-(cyclopropylmethyl)-3-[4-(7-methyl-4-morpholin-4-yl-7H-pyrrolo[2,3-h]quinazolin-2-yl)phenyl]urea;
1-(2-methoxyethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(tetrahydrofuran-2-ylmethyl)urea;
1-(2-cyclohex-1-en-1-ylethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(3-pyrrolidin-1-ylpropyl)urea;
1-cyclopentyl-3-[4-(7-methyl-4-morpholin-4-yl-7H-pyrrolo[2,3-h]quinazolin-2-yl)phenyl]urea;
1-cyclobutyl-3-[4-(7-methyl-4-morpholin-4-yl-7H-pyrrolo[2,3-h]quinazolin-2-yl)phenyl]urea;
1-cyclopropyl-3-[4-(7-methyl-4-morpholin-4-yl-7H-pyrrolo[2,3-h]quinazolin-2-yl)phenyl]urea;
1-cyclohexyl-3-[4-(7-methyl-4-morpholin-4-yl-7H-pyrrolo[2,3-h]quinazolin-2-yl)phenyl]urea;
propyl {4-[7-(methylsulfonyl)-4-morpholin-4-yl-7H-pyrrolo[2,3-h]quinazolin-2-yl]phenyl}carbamate;
1-methyl-3-{4-[7-(methylsulfonyl)-4-morpholin-4-yl-7H-pyrrolo[2,3-h]quinazolin-2-yl]phenyl}urea;
1-(4-{7-[2-(dimethylamino)ethyl]-4-morpholin-4-yl-7H-pyrrolo[2,3-h]quinazolin-2-yl}phenyl)-3-pyridin-3-ylurea;
N-(4-{7-[2-(dimethylamino)ethyl]-4-morpholin-4-yl-7H-pyrrolo[2,3-h]quinazolin-2-yl}phenyl)acetamide;
1-(4-{7-[2-(dimethylamino)ethyl]-4-morpholin-4-yl-7H-pyrrolo[2,3-h]quinazolin-2-yl}phenyl)-3-methylurea;
1-(3-acetylphenyl)-3-[4-(7-methyl-4-morpholin-4-yl-7H-pyrrolo[2,3-h]quinazolin-2-yl)phenyl]urea;
1-(4-acetylphenyl)-3-[4-(7-methyl-4-morpholin-4-yl-7H-pyrrolo[2,3-h]quinazolin-2-yl)phenyl]urea;
1-(3,5-dimethylisoxazol-4-yl)-3-[4-(7-methyl-4-morpholin-4-yl-7H-pyrrolo[2,3-h]quinazolin-2-yl)phenyl]urea;
1-(1,1-dioxidotetrahydrothiophen-3-yl)-3-[4-(7-methyl-4-morpholin-4-yl-7H-pyrrolo[2,3-h]quinazolin-2-yl)phenyl]urea;
1-(2-fluoroethyl)-3-[4-(7-methyl-4-morpholin-4-yl-7H-pyrrolo[2,3-h]quinazolin-2-yl)phenyl]urea;
1-[4-(7-methyl-4-morpholin-4-yl-7H-pyrrolo[2,3-h]quinazolin-2-yl)phenyl]-3-(2,2,2-trifluoroethyl)urea;
1-[4-(7-methyl-4-morpholin-4-yl-7H-pyrrolo[2,3-h]quinazolin-2-yl)phenyl]-3-(2-pyridin-4-ylethyl)urea;
1-{4-[7-(methylsulfonyl)-4-morpholin-4-yl-7H-pyrrolo[2,3-h]quinazolin-2-yl]phenyl}-3-propylurea;
1-[3-(7-methyl-4-morpholin-4-yl-7H-pyrrolo[2,3-h]quinazolin-2-yl)phenyl]-3-pyridin-4-ylurea;
1-[3-(7-methyl-4-morpholin-4-yl-7H-pyrrolo[2,3-h]quinazolin-2-yl)phenyl]-3-pyridin-3-ylurea;
ethyl [3-(7-methyl-4-morpholin-4-yl-7H-pyrrolo[2,3-h]quinazolin-2-yl)phenyl]carbamate;
N-[3-(7-methyl-4-morpholin-4-yl-7H-pyrrolo[2,3-h]quinazolin-2-yl)phenyl]cyclopropanecarboxamide;
N-[3-(7-methyl-4-morpholin-4-yl-7H-pyrrolo[2,3-h]quinazolin-2-yl)phenyl]butanamide;
1-[3-(7-methyl-4-morpholin-4-yl-7H-pyrrolo[2,3-h]quinazolin-2-yl)phenyl]-3-propylurea;
1-ethyl-3-[3-(7-methyl-4-morpholin-4-yl-7H-pyrrolo[2,3-h]quinazolin-2-yl)phenyl]urea;
N-[3-(7-methyl-4-morpholin-4-yl-7H-pyrrolo[2,3-h]quinazolin-2-yl)phenyl]propanamide;
methyl 4-({[4-(7-ethyl-4-morpholin-4-yl-7H-pyrrolo[2,3-h]quinazolin-2-yl)phenyl]carbamoyl}amino)benzoate;
1-[4-(7-ethyl-4-morpholin-4-yl-7H-pyrrolo[2,3-h]quinazolin-2-yl)phenyl]-3-{4-[(4-methylpiperazin-1-yl)carbonyl]phenyl}urea;
4-({[4-(7-ethyl-4-morpholin-4-yl-7H-pyrrolo[2,3-h]quinazolin-2-yl)phenyl]carbamoyl}amino)-N-[2-(methylamino)ethyl]benzamide;
N-[2-(dimethylamino)ethyl]-4-({[4-(7-ethyl-4-morpholin-4-yl-7H-pyrrolo[2,3-h]quinazolin-2-yl)phenyl]carbamoyl}amino)-N-methylbenzamide; and
1-(4-{[4-(dimethylamino)piperidin-1-yl]carbonyl}phenyl)-3-[4-(7-ethyl-4-morpholin-4-yl-7H-pyrrolo[2,3-h]quinazolin-2-yl)phenyl]urea.

13. A composition comprising the compound of claim 1, and a pharmaceutically acceptable carrier.

14. A composition of claim 13 comprising a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, lavendustin A, hydroxyzine, glatiramer acetate, interferon beta-1a, interferon beta-1b, natalizumab and lavendustin A; and a pharmaceutically acceptable carrier.

15. A composition of claim 14 wherein, the second compound is Avastin.

16. A method of treating a PI3K-related disorder, an mTOR-related disorder, or a hSMG-1-related disorder comprising administering to a mammal in need thereof an effective amount of a compound of claim 1.

17. The method of claim 16 wherein, the PI3K-related disorder, mTOR-related disorder, or hSMG-1-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

18. The method of claim 17 wherein, the PI3K-related disorder, mTOR-related disorder, or hSMG-1-related disorder is cancer.

19. The method of claim 18 wherein, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

20. A method of treating advanced renal cell carcinoma, acute lymphoblastic leukemia, acute malignant melanoma, soft-tissue or bone sarcoma, comprising administering to a mammal in need thereof an effective amount of a compound of claim 1.

21. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition of claim 15 in an amount effective to treat the cancer.

22. A method of inhibiting mTOR, PI3K, or hSMG-1 in a subject, comprising administering to a subject in need thereof a compound of claim 1 in an amount effective to inhibit mTOR, PI3K, or hSMG-1.

23. A method of inhibiting mTOR, PI3K, and hSMG-1 together in a subject, comprising administering to a subject in need thereof a compound of claim 1 in an amount effective to inhibit mTOR, PI3K, and hSMG-1.

24. A method of synthesizing compounds of the claim 2 comprising: reacting a boronic acid of the formula Rₙ¹—Ar—B(OH)₂ with the 2-chloro-7H-pyrrolo[2,3-h]quinazoline 24: 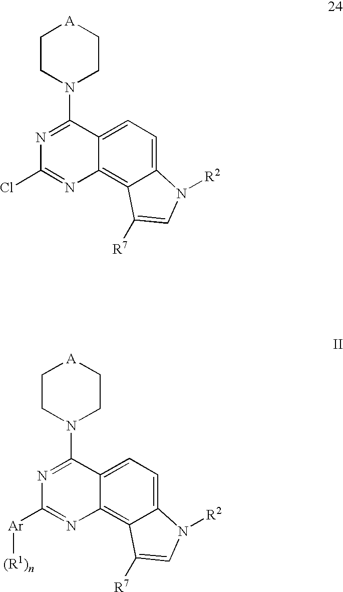
to give the 7H-pyrrolo[2,3-h]quinazoline II.

25. A method of synthesizing compounds of the formula 23 comprising reacting the 7H-pyrrolo[2,3-h]quinazoline of Formula 17 with an alkylating or acylating agent R²—X to substitute the amino group at position 7 of the 7H-pyrrolo[2,3-h]quinazoline, wherein X is halogen, 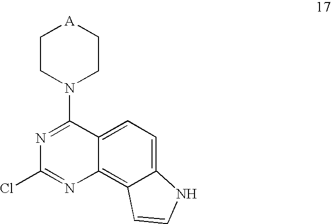
under conditions effective to alkylate or acylate the nitrogen atom at position 7 of the pyrrole ring thereby producing 23, wherein A is —O—, —CH₂O—, or —S(O)ₘ—; m is 0, 1, or 2; and R² is R² is C₁-C₆alkyl optionally substituted with from 1 to 3 substituents independently selected from —NH₂, —NH(C₁-C₆alkyl), —N(C₁-C₆alkyl)(C₁-C₆alkyl), C₆-C₁₄aryl, and —C(O)O(C₁-C₆alkyl); —S(O)_{q}—(C₁-C₆alkyl); —S(O)_{q}—(C₁-C₉heteroaryl); —S(O)_{q}—(C₆-C₁₄aryl); or —S(O)_{q}-(4- to 7-membered monocyclic heterocycle group) optionally substituted with from 1 to 3 substituents independently selected from C₁-C₆alkyl and (C₆-C₁₄aryl)alkyl-O—C(O)—; 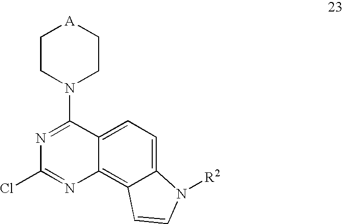
optionally reacting 23 with a formylating agent under Vilsmeier-Haack conditions to formylate the pyrrole ring, thereby producing the chlorinated intermediate 24, 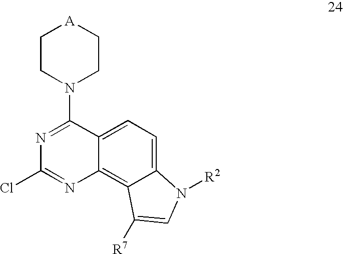
wherein R⁷ is CHO, under conditions effective to replace the hydrogen atom at position 9 of the 7H-pyrrolo[2,3-h]quinazoline.

26. A compound of the Formula III: 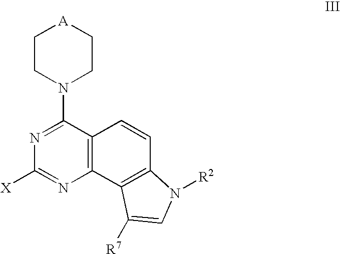
wherein A is —O—, —CH₂O—, or S(O)ₘ;
m is 0, 1, or 2;
X is halogen;
and the remaining variables are as defined in claim 1.

27. A compound selected from the group consisting of:
benzyl 4-[(2-chloro-4-morpholin-4-yl-7H-pyrrolo[2,3-h]quinazolin-7-yl)sulfonyl]piperidine-1-carboxylate;
2-chloro-4-morpholin-4-yl-7-(phenylsulfonyl)-7H-pyrrolo[2,3-h]quinazoline;
2-chloro-7-(methylsulfonyl)-4-morpholin-4-yl-7H-pyrrolo[2,3-h]quinazoline;
2-chloro-7-(2-(dimethylamino)ethyl)-4-morpholin-4-yl-7H-pyrrolo[2,3h]quinazoline; and
2-chloro-7-methyl-4-morpholin-4-yl-7H-pyrrolo[2,3-h]quinazoline.
